(19) United States
(12) Patent Application Publication
(10) Pub. No.: US 2023/0348454 A1
(43) Pub. Date: Nov. 2, 2023

(54) COMPOUNDS AS MODULATORS OF BIS-PHOSPHOGLYCERATE MUTASE FOR THE TREATMENT OF SICKLE CELL DISEASE

(71) Applicant: Genzyme Corporation, Cambridge, MA (US)

(72) Inventors: Kunal Desai, Waltham, MA (US); Zhong Fang, Waltham, MA (US); Kevin Guertin, Waltham, MA (US); Vu Hong, Cambridge, MA (US); John Ziqi Jiang, Waltham, MA (US); Sungtaek Lim, Waltham, MA (US); Jinyu Liu, Waltham, MA (US); Mark Munson, Natick, MA (US)

(21) Appl. No.: 18/025,853

(22) PCT Filed: Sep. 14, 2021
(86) PCT No.: PCT/US2021/050216

§ 371 Date: Mar. 10, 2023

Related U.S. Application Data
(60) Provisional application No. 63/077,973, filed on Sep. 14, 2020.

(51) Int. Cl. C07D 417/14; C07D 417/04

(52) U.S. Cl. CPC C07D 417/14; C07D 417/04

(57) ABSTRACT

Provided herein are compounds and compositions thereof for modulating bis-phosphoglycerate mutase (BPGM) for treating sickle cell disease.

### CROSS-REFERENCE TO RELATED APPLICATIONS

This application claims the benefit under 35 U.S.C. § 119(e) of U.S. Provisional Application No. 63/077,973, filed Sep. 14, 2020, which is hereby incorporated by reference in its entirety.

### FIELD

The present disclosure relates generally to compounds, compositions, and method for treating sickle cell disease.

### BACKGROUND

Bis-phosphoglycerate mutase (BPGM) is an enzyme in the glucose metabolism pathway that regulates the levels of 2,3-bis-phosphoglycerate (2,3-BPG) inside the red blood cell (RBC). 2,3-BPG is a known allosteric modulator of hemoglobin that stabilizes the de-oxy or “T-State” of hemoglobin that has a tendency to polymerize, resulting in the sickle cell morphology. There is currently a large unmet medical need for safe and effective oral therapies for the treatment of sickle cell disease. BPGM modulators that lower 2,3-BPG levels offer a novel mechanism of action from existing therapies and may significantly reduce sickling in sickle cell disease patients.

Accordingly, in one aspect, provided herein are compounds which are modulators of BPGM for use in treating sickle cell disease.

### SUMMARY

Described herein, in certain embodiments, are compounds and compositions thereof for modulating bis-phosphoglycerate mutase (BPGM) for treating sickle cell disease.

The following embodiments are encompassed.

Embodiment 1a is a compound of Formula (I):

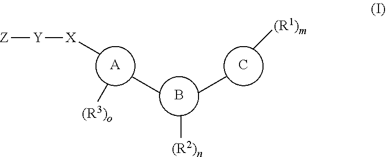

or a pharmaceutically acceptable salt thereof, wherein:
Ring A is a 5-membered heteroarylene containing 1-3 heteroatoms selected from N and S;
Ring B is phenylene or a 5- to 6-membered heteroarylene containing 1-3 heteroatoms selected from N and S;
Ring C is a fused bicyclic 9- to 10-membered heteroaryl or heterocyclyl containing 1-4 heteroatoms selected from N and O, a 5- to 6-membered heteroaryl containing 1-4 nitrogen atoms, or phenyl substituted by one R⁴;
each R¹ is independently —OH, halo, oxo, C₁-C₆ alkyl, C₁-C₆ haloalkyl, —CN, —N(H)C(O)R⁶, —N(H)SO₂R⁶, —SO₂NR⁶R⁷, or —C(O)N(H)SO₂R⁶;
each R² is independently C₁-C₆ alkyl, C₁-C₆ haloalkyl, or halo;
each R³ is independently C₁-C₆ alkyl, C₁-C₆ haloalkyl, or C₃-C₆ cycloalkyl;
R⁴ is —OH, —OP(O)(OH)₂, —NO₂, —C(O)N(H)SO₂R⁵, 5- to 6-membered heteroaryl or heterocyclyl, or —C(O)N(H)-(5- to 6-membered heteroaryl or heterocyclyl),
   wherein the heteroaryl and heterocyclyl contain 1-4 heteroatoms selected from N, O, and S, and wherein the heterocyclyl is optionally substituted by 1-2 oxo groups;
m is 0-4;
n is 0-2;
o is 0-2;
R⁵ is phenyl or C₁-C₆ alkyl;
X is —CR⁶R⁷— or a bond;
Y is —O—, —N(H)—, or a bond;
each R⁶ and R⁷ is independently H or C₁-C₆ alkyl;
Z is Z¹ or Z²;
Z¹ is H, C₁-C₆ alkyl, —C(O)(C₁-C₆ alkyl), —CO₂(C₁-C₆ alkyl), —C(O)NR⁶R⁷, —C(O)NR⁶(C₁-C₆ alkylene)NR⁶R⁷, —CR⁶R⁷C(O)NR⁶R⁷, —CR⁶R⁷NR⁶R⁷, —C(O)(C₁-C₆ alkylene)NR⁶R⁷, —NR⁶C(O)(C₁-C₆ alkyl), —NR⁶R⁷, —(C₁-C₆ alkyl)-CO₂H, —(C₁-C₆ alkyl)-OH, or —C(NR⁶R⁷)═N—CN, wherein C₁-C₆ alkylene is optionally substituted by 1-6 halo, C₁-C₆ alkyl, or C₁-C₆ haloalkyl;
Z² is 5- to 6-membered heteroaryl, —C(O)(5- to 6-membered heteroaryl), —CH₂C(O)(5- to 6-membered heteroaryl), 4- to 6-membered heterocyclyl, —C(O)(4- to 6-membered heterocyclyl), or —CH₂C(O)(4- to 6-membered heterocyclyl), wherein the heteroaryl and heterocyclyl contain 1-3 heteroatoms selected from N and O, and wherein the heteroaryl and heterocyclyl are optionally substituted by 1-3 R⁸;
each R⁸ is independently halo, C₁-C₆ alkyl, C₁-C₆ haloalkyl, —NR⁶R⁷, —OH, oxo, —CO₂H, —O(C₁-C₆ alkyl), —CH₂—O(C₁-C₆ alkyl), or —(C₁-C₆ alkyl)-OH.

Embodiment 1b is a compound of Formula (I):

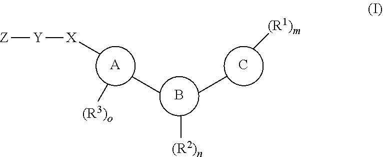

or a pharmaceutically acceptable salt thereof, wherein:
Ring A is a 5-membered heteroarylene containing 1-3 heteroatoms selected from N and S;
Ring B is phenylene or a 5- to 6-membered heteroarylene containing 1-3 heteroatoms selected from N and S;
Ring C is a fused bicyclic 9- to 10-membered heteroaryl or heterocyclyl containing 1-4 heteroatoms selected from N and O, a 5- to 6-membered heteroaryl containing 1-4 nitrogen atoms, or phenyl substituted by one R⁴;
each R¹ is independently —OH, halo, oxo, C₁-C₆ alkyl, C₁-C₆ haloalkyl, —CN, —N(H)C(O)R⁶, —N(H)SO₂R⁶, —N(H)SO₂(C₆H₅), —SO₂NR⁶R⁷, or —C(O)N(H)SO₂R⁶;
each R² is independently C₁-C₆ alkyl, C₁-C₆ haloalkyl, or halo;
each R³ is independently C₁-C₆ alkyl, C₁-C₆ haloalkyl, or C₃-C₆ cycloalkyl;
R⁴ is —OH, —OP(O)(OH)₂, —NO₂, —C(O)N(H)SO₂R⁵, 5- to 6-membered heteroaryl or heterocyclyl, or —C(O)N(H)-(5- to 6-membered heteroaryl or heterocyclyl),
   wherein the heteroaryl and heterocyclyl contain 1-4 heteroatoms selected from N, O, and S, and wherein the heterocyclyl is optionally substituted by 1-2 oxo groups;
m is 0-4;
n is 0-2;
o is 0-2;
R⁵ is phenyl or C₁-C₆ alkyl;
X is —CR⁶R⁷— or a bond;
Y is —O—, —N(H)—, or a bond;
each R⁶ and R⁷ is independently H or C₁-C₆ alkyl;
Z is Z¹ or Z²;
Z¹ is H, C₁-C₆ alkyl, —C(O)(C₁-C₆ alkyl), —CO₂(C₁-C₆ alkyl), —C(O)NR⁶R⁷, —C(O)NR⁶(C₁-C₆ alkylene)NR⁶R⁷, —CR⁶R⁷C(O)NR⁶R⁷, —CR⁶R⁷NR⁶R⁷, —C(O)(C₁-C₆ alkylene)NR⁶R⁷, —NR⁶C(O)(C₁-C₆ alkyl), —NR⁶R⁷, —(C₁-C₆ alkyl)-CO₂H, —(C₁-C₆ alkyl)-OH, or —C(NR⁶R⁷)═N—CN, wherein C₁-C₆ alkylene is optionally substituted by 1-6 halo, C₁-C₆ alkyl, or C₁-C₆ haloalkyl;
Z² is 5- to 6-membered heteroaryl, —C(O)(5- to 6-membered heteroaryl), —CH₂C(O)(5- to 6-membered heteroaryl), 4- to 6-membered heterocyclyl, —C(O)(4- to 6-membered heterocyclyl), or —CH₂C(O)(4- to 6-membered heterocyclyl), wherein the heteroaryl and heterocyclyl contain 1-3 heteroatoms selected from N and O, and wherein the heteroaryl and heterocyclyl are optionally substituted by 1-3 R⁸;
each R⁸ is independently halo, C₁-C₆ alkyl, C₁-C₆ haloalkyl, —NR⁶R⁷, —OH, oxo, —CO₂H, —O(C₁-C₆ alkyl), —CH₂—O(C₁-C₆ alkyl), or —(C₁-C₆ alkyl)-OH.

Embodiment 2 is the compound of embodiment 1, or a pharmaceutically acceptable salt thereof, wherein:
Ring A is thiazolylene.

Embodiment 3 is the compound of embodiment 1 or 2, or a pharmaceutically acceptable salt thereof, wherein:
each R³ is independently C₁-C₃ alkyl or C₃-C₆ cycloalkyl.

Embodiment 4 is the compound of embodiment 3, or a pharmaceutically acceptable salt thereof, wherein:
each R³ is independently —CH₃ or cyclopropyl.

Embodiment 5 is the compound of any one of embodiments 1-4, or a pharmaceutically acceptable salt thereof, wherein:
o is 0 or 1.

Embodiment 6 is the compound of any one of embodiments 1-5, or a pharmaceutically acceptable salt thereof, wherein:
Ring B is phenylene, pyridinylene, thiazolylene, pyrazolylene, pyrimidinylene, or thienylene.

Embodiment 7 is the compound of any one of embodiments 1-6, or a pharmaceutically acceptable salt thereof, wherein:
each R² is independently C₁-C₃ alkyl or halo.

Embodiment 8 is the compound of any one of embodiments 1-7, or a pharmaceutically acceptable salt thereof, wherein:
n is 0 or 1.

Embodiment 9 is the compound of any one of embodiments 1-8, or a pharmaceutically acceptable salt thereof, wherein:
Ring C is a fused bicyclic 9-membered heteroaryl or heterocyclyl containing 1-4 heteroatoms selected from N and O, a 5- to 6-membered heteroaryl containing 1-4 nitrogen atoms, or phenyl substituted by one R⁴.

Embodiment 10 is the compound of any one of embodiments 1-9, or a pharmaceutically acceptable salt thereof, wherein:
R⁴ is —OH, —OP(O)(OH)₂, —NO₂, —C(O)N(H)SO₂R⁵, 5- to 6-membered heteroaryl or heterocyclyl, or —C(O)N(H)-(5- to 6-membered heteroaryl or heterocyclyl),
   wherein the heteroaryl and heterocyclyl contain 1-4 heteroatoms selected from N, O, and S, and wherein the heterocyclyl is optionally substituted by 1-2 oxo groups; and
R⁵ is phenyl or C₁-C₃ alkyl.

Embodiment 11 is the compound of any one of embodiments 1-10, or a pharmaceutically acceptable salt thereof, wherein Ring C—(R¹)ₘ is selected from

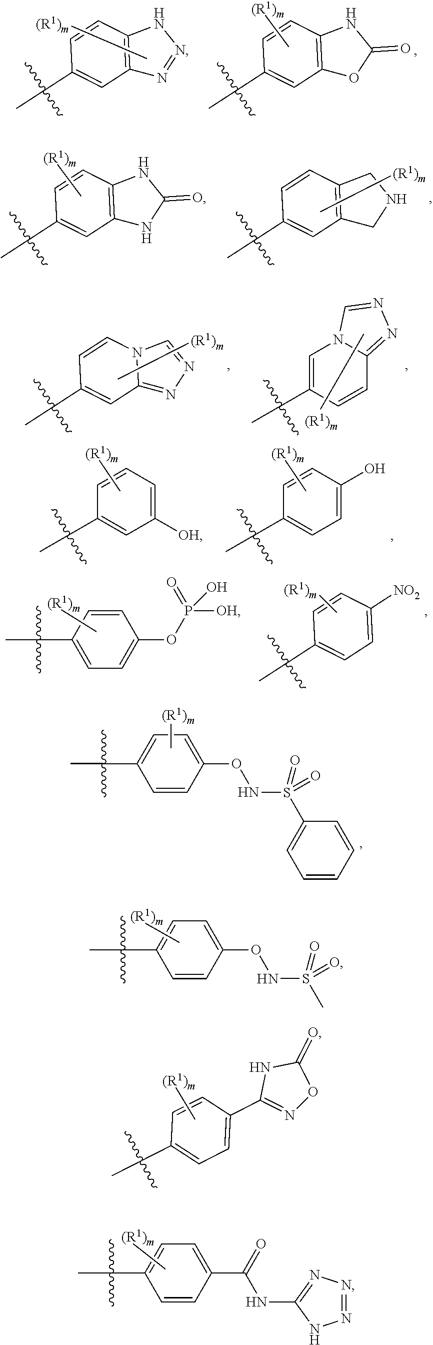

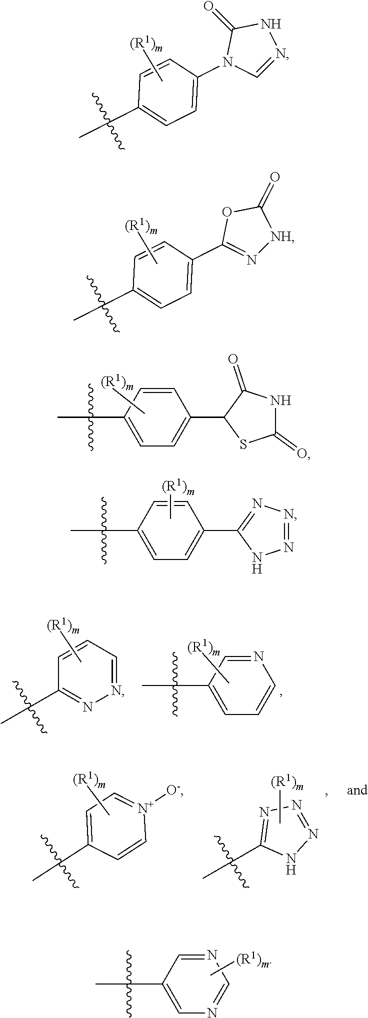

Embodiment 12a is the compound of any one of embodiments 1-11, or a pharmaceutically acceptable salt thereof, wherein:
each R¹ is independently —OH, halo, oxo, C₁-C₃ alkyl, C₁-C₃ haloalkyl, —CN, —N(H)C(O)R⁶, —N(H)SO₂R⁶, —SO₂NR⁶R⁷, or —C(O)N(H)SO₂R⁶; and
each R⁶ and R⁷ is independently H or C₁-C₃ alkyl.

Embodiment 12b is the compound of any one of embodiments 1-11, or a pharmaceutically acceptable salt thereof, wherein:
each R¹ is independently —OH, halo, oxo, C₁-C₃ alkyl, C₁-C₃ haloalkyl, —CN, —N(H)C(O)R⁶, —N(H)SO₂R⁶, —N(H)SO₂(C₆H₅), —SO₂NR⁶R⁷, or —C(O)N(H)SO₂R⁶; and
each R⁶ and R⁷ is independently H or C₁-C₃ alkyl.

Embodiment 13 is the compound of embodiment 12, or a pharmaceutically acceptable salt thereof, wherein:
each R¹ is independently —OH, F, oxo, —CH₃, —CN, —N(H)C(O)CH₃, —N(H)SO₂(C₆H₅), —N(H)SO₂CH₃, —SO₂NH₂, or —C(O)N(H)SO₂CH₃.

Embodiment 14 is the compound of any one of embodiments 1-13, or a pharmaceutically acceptable salt thereof, wherein:
m is 0-3.

Embodiment 15 is the compound of any one of embodiments 1-14, or a pharmaceutically acceptable salt thereof, wherein:
X is —CR⁶R⁷—.

Embodiment 16 is the compound of embodiment 15, or a pharmaceutically acceptable salt thereof, wherein:
R⁶ and R⁷ are each H.

Embodiment 17 is the compound of any one of embodiments 1-14, or a pharmaceutically acceptable salt thereof, wherein:
X is a bond.

Embodiment 18 is the compound of any one of embodiments 1-17, or a pharmaceutically acceptable salt thereof, wherein:
Y is —O—.

Embodiment 19 is the compound of any one of embodiments 1-17, or a pharmaceutically acceptable salt thereof, wherein:
Y is —N(H)— or a bond.

Embodiment 20 is the compound of any one of embodiments 1-19, or a pharmaceutically acceptable salt thereof, wherein:
Z is Z¹.

Embodiment 21 is the compound of embodiment 20, or a pharmaceutically acceptable salt thereof, wherein:
Z¹ is H, C₁-C₃ alkyl, —C(O)(C₁-C₃ alkyl), —CO₂(C₁-C₃ alkyl), —C(O)NR⁶R⁷, —C(O)NR⁶(C₁-C₃ alkylene)NR⁶R⁷, —CR⁶R⁷C(O)NR⁶R⁷, —CR⁶R⁷NR⁶R⁷, —C(O)(C₁-C₃ alkylene)NR⁶R⁷, —NR⁶C(O)(C₁-C₃ alkyl), —NR⁶R⁷, —(C₁-C₃ alkyl)-CO₂H, —(C₁-C₃ alkyl)-OH, or —C(NR⁶R⁷)═N—CN, wherein C₁-C₃ alkylene is optionally substituted by 1-2 halo, C₁-C₃ alkyl, or C₁-C₃ haloalkyl; and
each R⁶ and R⁷ is independently H or C₁-C₃ alkyl.

Embodiment 22 is the compound of embodiment 20 or 21, or a pharmaceutically acceptable salt thereof, wherein:
Z¹ is H, —CO₂CH₃, —CO₂C(CH₃)₃, —C(O)CH₃, —C(O)C(CH₃)₃, —C(N(CH₃)₂)═N—CN, —C(O)N(CH₃)₂, —CH₃, —CH₂CH₃, —CH₂C(CH₃)₃, —CH₂CO₂H, —CH₂C(CH₃)₂OH, —C(O)N(CH₃)CH₂CH₂N(CH₃)₂, —CH₂C(O)N(CH₃)₂, —CH(CH₃)C(O)N(CH₃)₂, —N(CH₃)₂, —N(CH₃)C(O)CH₃, or —C(O)CH(CH₃)CH₂N(CH₃)₂.

Embodiment 23 is the compound of any one of embodiments 1-19, or a pharmaceutically acceptable salt thereof, wherein:
Z is Z².

Embodiment 24 is the compound of embodiment 23, or a pharmaceutically acceptable salt thereof, wherein:
Z² is 5- to 6-membered heteroaryl, —C(O)(5- to 6-membered heteroaryl), —CH₂C(O)(5- to 6-membered heteroaryl), 4- to 6-membered heterocyclyl, —C(O)(4- to 6-membered heterocyclyl), or —CH₂C(O)(4- to 6-membered heterocyclyl), wherein the heteroaryl and heterocyclyl contain 1-2 heteroatoms selected from N and O, and wherein the heteroaryl and heterocyclyl are optionally substituted by 1-3 R⁸.

Embodiment 25 is the compound of embodiment 23 or 24, or a pharmaceutically acceptable salt thereof, wherein:
Z² is pyrimidinyl, pyridazinyl, pyrrolidinyl, piperidinyl, pyridazinyl, —C(O)(tetrahydropyranyl), —C(O)(pyrrolidinyl), —C(O)(pyridazinyl), —C(O)(piperidinyl), —C(O)(azetidinyl), —C(O)(pyrazolyl), —C(O)(piperazinyl), —CH₂C(O)(pyrrolidinyl), —CH₂C(O)(piperidinyl), or —CH₂C(O)(piperazinyl), wherein the heteroaryl and heterocyclyl are optionally substituted by 1-3 R⁸.

Embodiment 26 is the compound of any one of embodiments 1-19 and 23-25, or a pharmaceutically acceptable salt thereof, wherein:
each R⁸ is independently halo, C₁-C₃ alkyl, C₁-C₃ haloalkyl, —NR⁶R⁷, —OH, oxo, —CO₂H, —O(C₁-C₃ alkyl), —CH₂—O(C₁-C₃ alkyl), or —(C₁-C₃ alkyl)-OH; and
each R⁶ and R⁷ is independently H or C₁-C₃ alkyl.

Embodiment 27 is the compound of embodiment 26, or a pharmaceutically acceptable salt thereof, wherein:
each R⁸ is independently —CH₃, —CH₂CH₃, —NH₂, —OH, oxo, —N(CH₃)₂, —OCH₃, —CH₂OCH₃, —CF₃, —CH₂CH₂OH, —CO₂H, or Cl.

Embodiment 28 is the compound of any one of embodiments 1-22, or a pharmaceutically acceptable salt thereof, wherein:
Z—Y—X— is

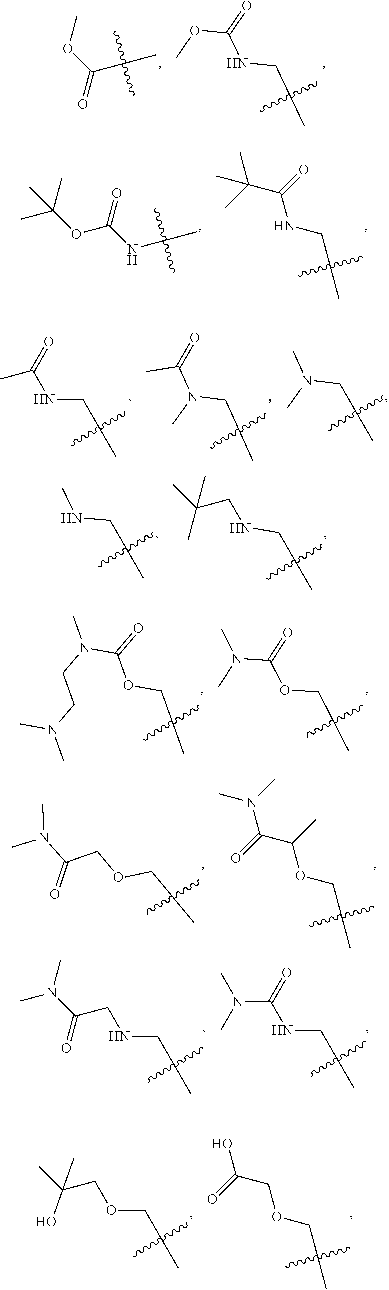

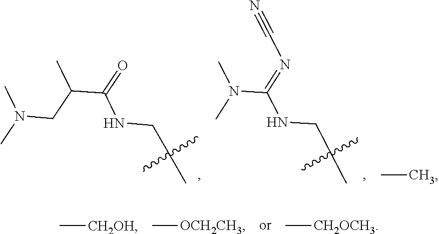

Embodiment 29 is the compound of any one of embodiments 1-19 and 23-27, or a pharmaceutically acceptable salt thereof, wherein:
Z—Y—X— is

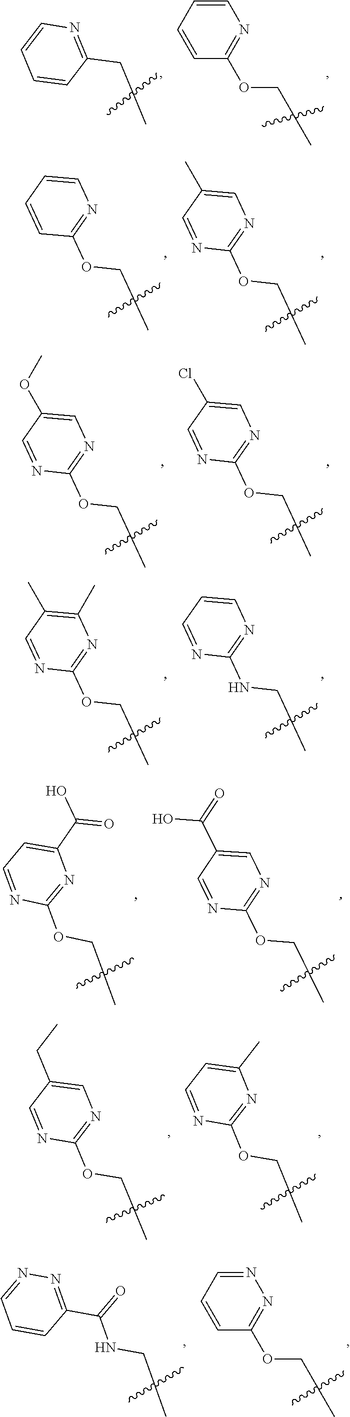

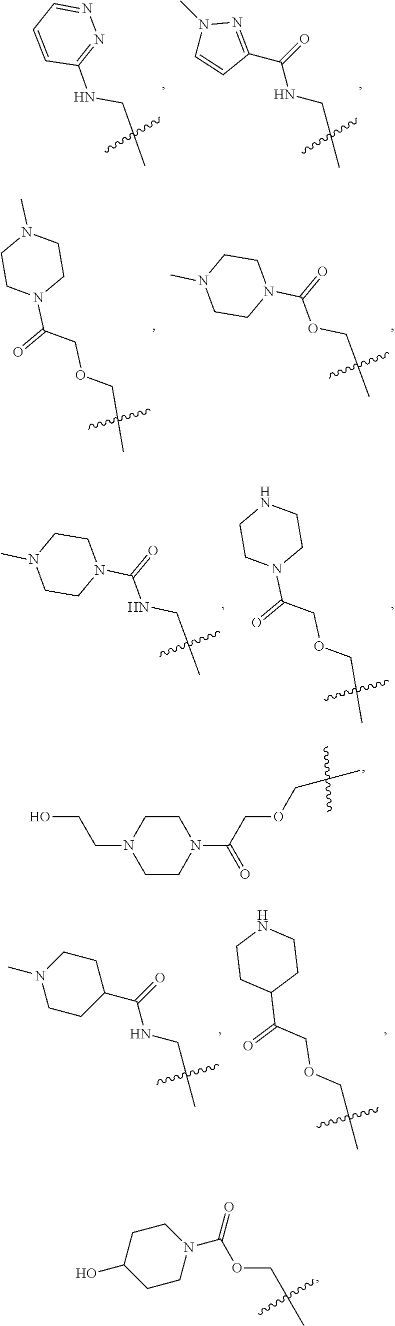

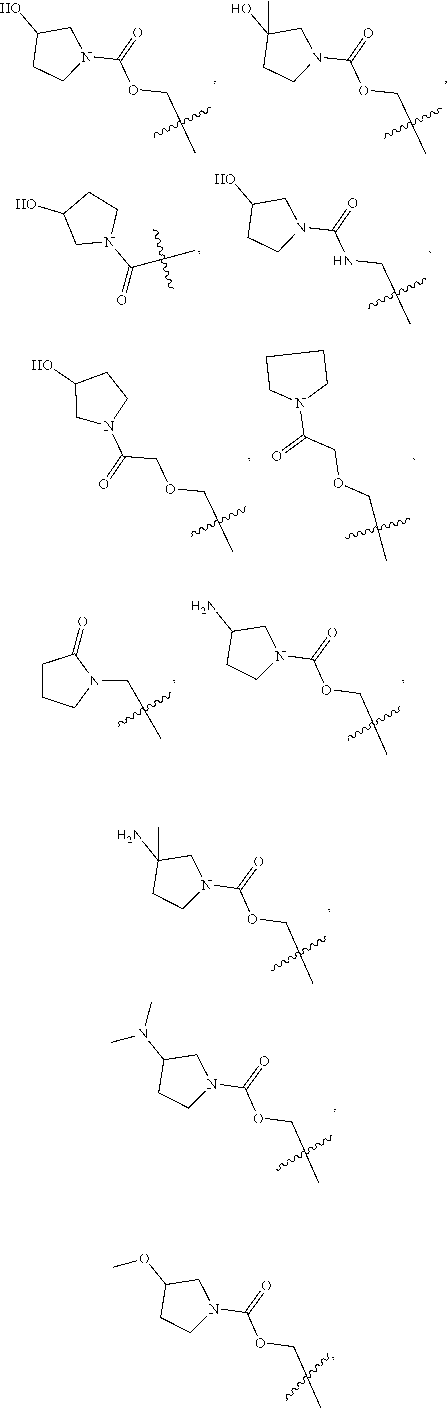

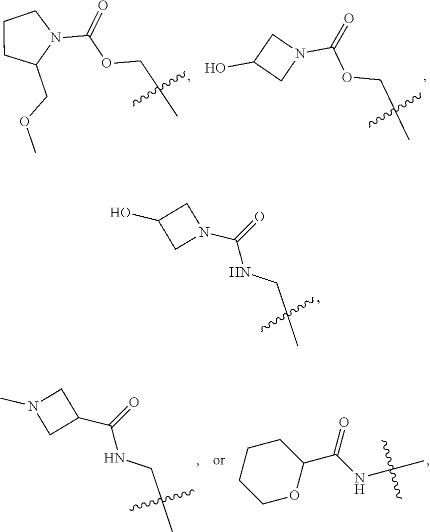

Embodiment 30 is a compound selected from the compounds in Table 1, or a pharmaceutically acceptable salt thereof.

Embodiment 31 is a compound selected from the compounds in Table 2, or a pharmaceutically acceptable salt thereof.

Embodiment 32 is a pharmaceutical composition comprising the compound of any one of embodiments 1-31, or a pharmaceutically acceptable salt thereof, and at least one pharmaceutically acceptable excipient.

Embodiment 33 is a method of modulating bis-phosphoglycerate mutase (BPGM) comprising contacting an effective amount of the compound of any one of embodiments 1-31, or a pharmaceutically acceptable salt thereof, or the pharmaceutical composition of embodiment 32, with the BPGM.

Embodiment 34 is a method of treating sickle cell disease in a subject in need thereof, comprising administering to the subject a therapeutically effective amount of the compound of any one of embodiments 1-31, or a pharmaceutically acceptable salt thereof, or the pharmaceutical composition of embodiment 32.

### DETAILED DESCRIPTION

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. To the extent any material incorporated herein by reference is inconsistent with the express content of this disclosure, the express content controls. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the disclosure.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, such as for example, within 15%, 10%, or 5%.

The section headings used herein are for organizational purposes only and are not to be construed as limiting the subject matter described.

“Alkyl” refers to an unbranched or branched saturated hydrocarbon chain. As used herein, alkyl has 1 to 20 carbon atoms (i.e., C₁-C₂₀ alkyl), 1 to 10 carbon atoms (i.e., C₁-C₁ alkyl), 1 to 6 carbon atoms (i.e., C₁-C₆ alkyl) or 1 to 3 carbon atoms (i.e., C₁-C₃ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), isobutyl (i.e., —CH₂CH(CH₃)₂), sec-butyl (i.e., —CH(CH₃)CH₂CH₃), and tert-butyl (i.e., —C(CH₃)₃); and “propyl” includes n-propyl (i.e., —(CH₂)₂CH₃) and isopropyl (i.e., —CH(CH₃)₂).

“Alkyl-OH” refers to an refers to an unbranched or branched alkyl group as defined above, wherein one or more hydrogen atoms are replaced by a hydroxyl (—OH). For example, “(C₁-C₆ alkyl)-OH” refers to a C₁-C₆ alkyl which is substituted by one or more hydroxyl groups. A C₁ alkyl-OH refers to a methyl group that may be substituted by 1-3 hydroxyl groups, a C₂ alkyl-OH refers to an ethyl group that may be substituted by 1-5 hydroxyl groups, a C₃ alkyl-OH refers to a propyl group that may be substituted by 1-7 hydroxyl groups, etc. Examples of alkyl-OH include —CH₂OH, —CH₂CH₂OH, —CH₂CH(OH)CH₂(OH), and the like.

“Alkyl-CO₂H” refers to an refers to an unbranched or branched alkyl group as defined above, wherein one or more hydrogen atoms are replaced by a carboxylic acid (—CO₂H). For example, “(C₁-C₆ alkyl)-CO₂H” refers to a C₁-C₆ alkyl which is substituted by one or more carboxylic acid groups. A C₁ alkyl-CO₂H refers to a methyl group that may be substituted by 1-3 carboxylic acid groups, a C₂ alkyl-CO₂H refers to an ethyl group that may be substituted by 1-5 carboxylic acid groups, a C₃ alkyl-CO₂H refers to a propyl group that may be substituted by 1-7 carboxylic acid groups, etc. Examples of alkyl-CO₂H include —CH₂CO₂H, —CH₂CH₂CO₂H, —CH₂CH(CO₂H)CH₃, —CH₂CH(CO₂H)CH₂CO₂H, and the like.

“Haloalkyl” refers to an unbranched or branched alkyl group as defined above, wherein one or more hydrogen atoms are replaced by a halogen. For example, “C₁-C₆ haloalkyl” refers to a C₁-C₆ alkyl which is substituted by one or more halogen atoms. A C₁ haloalkyl refers to a methyl group that may be substituted by 1-3 halo groups, a C₂ haloalkyl refers to an ethyl group that may be substituted by 1-5 halo groups, a C₃ haloalkyl refers to a propyl group that may be substituted by 1-7 halo groups, etc. Examples of haloalkyl include trifluoromethyl, difluoromethyl, trichloromethyl, 2,2,2-trifluoroethyl, 1,2-difluoroethyl, 3-bromo-2-fluoropropyl, 1,2-dibromoethyl, and the like. A haloalkyl may contain one or more halo atoms that are the same (i.e., all fluoro) or a mixture of halo atoms (i.e, chloro and fluoro).

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond). As used herein, cycloalkyl has from 3 to 20 ring carbon atoms (i.e., C₃-C₂₀ cycloalkyl), 3 to 10 ring carbon atoms (i.e., C₃-C₁₀ cycloalkyl), or 3 to 6 ring carbon atoms (i.e., C₃-C₆ cycloalkyl). Cycloalkyl also includes “spiro cycloalkyl” when there are two positions for substitution on the same carbon atom. Monocyclic radicals include, for example, cyclopropyl, cyclobutyl, cyclopentyl, cyclohexyl, cycloheptyl and cyclooctyl. Polycyclic radicals include, for example, adamantyl, norbornyl, decalinyl, 7,7-dimethyl-bicyclo[2.2.1]heptanyl and the like. Further, the term cycloalkyl is intended to encompass any non-aromatic ring which may be fused to an aryl ring, regardless of the attachment to the remainder of the molecule.

“Halogen” or “halo” includes fluoro, chloro, bromo, and iodo.

“Oxo” refers to the atom (═O) or (O).

“Heteroaryl” refers to an aromatic group (e.g., a 5-14 membered ring system) having a single ring, multiple rings, or multiple fused rings, with one or more ring heteroatoms independently selected from nitrogen, oxygen and sulfur. As used herein, heteroaryl includes 1 to 10 ring carbon atoms and 1 to 4 heteroatoms independently selected from nitrogen, oxygen and sulfur within the ring. Examples of heteroaryl groups include pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl and thiophenyl (i.e., thienyl). A heteroaryl may comprise one or more N-oxide (N—O—) moieties, such as pyridine-N-oxide.

“Heterocyclyl” refers to a saturated or unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro, and may comprise one or more oxo (C═O) or N-oxide (N—O—) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 1 to 10 ring carbon atoms, 1 to 8 carbon atoms, 1 to 6 carbon atoms, or 1 to 4 carbon atoms, and 1 to 5 ring heteroatoms, 1 to 4 heteroatoms, 1 to 3 heteroatoms, or 1 to 2 heteroatoms independently selected from nitrogen, sulfur and oxygen. Examples of heterocyclyl groups include dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, 1-oxo-thiomorpholinyl, and 1,1-dioxo-thiomorpholinyl.

Certain commonly used alternative chemical names may be used. For example, a divalent group such as a divalent “alkyl” group, a divalent “phenyl” group, a divalent “heteroaryl” group, a divalent “heterocyclyl” group etc., may also be referred to as an “alkylene” group, an “phenylene” group, a “heteroarylene” group, or a “heterocyclylene” group, respectively.

The terms “optional” or “optionally” means that the subsequently described event or circumstance may or may not occur and that the description includes instances where said event or circumstance occurs and instances in which it does not. Also, the term “optionally substituted” refers to any one or more hydrogen atoms on the designated atom or group may or may not be replaced by a moiety other than hydrogen.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan.

Any compound or Formula described herein is intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I and ¹²⁵I, respectively. Various isotopically labeled compounds of the present disclosure, for example those into which radioactive isotopes such as ³H and ¹⁴C are incorporated, are included in this disclosure. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes “deuterated analogs” of compounds described herein in which from 1 to n hydrogens attached to a carbon atom is/are replaced by deuterium, in which n is the number of hydrogens in the molecule. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

“Pharmaceutically acceptable” refers to compounds, salts, compositions, dosage forms and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” include, for example, salts with inorganic acids and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts. Pharmaceutically acceptable acid addition salts may be prepared from inorganic and organic acids. Salts derived from inorganic acids include hydrochloric acid, hydrobromic acid, sulfuric acid, nitric acid, phosphoric acid and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid and the like. Likewise, pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine and the like.

The compounds disclosed herein, or their pharmaceutically acceptable salts, may include an asymmetric center and may thus give rise to enantiomers, diastereomers, and other stereoisomeric forms that may be defined, in terms of absolute stereochemistry, as (R)- or (S)- or, as (D)- or (L)- for amino acids. The disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

“Tautomer” refers to alternate forms of a compound that differ in the position of a proton, such as enol-keto and imine-enamine tautomers, or the tautomeric forms of heteroaryl groups containing a ring atom attached to both a ring —NH-moiety and a ring=N moiety such as pyrazoles, imidazoles, benzimidazoles, triazoles, and tetrazoles. All tautomeric forms of the compounds described herein are intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are nonsuperimposable mirror images of one another.

“Diastereoisomers” are stereoisomers that have at least two asymmetric atoms, but which are not mirror-images of each other.

As used herein, “pharmaceutically acceptable carrier” or “pharmaceutically acceptable excipient” or “excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Effective amount” or dose of a compound or a composition refers to that amount of the compound or the composition that results in an intended result as desired based on the disclosure herein. Effective amounts can be determined by standard pharmaceutical procedures in cell cultures or experimental animals including, without limitation, by determining the LD₅₀ (the dose lethal to 50% of the population) and the ED₅₀ (the dose therapeutically effective in 50% of the population).

“Therapeutically effective amount” or dose of a compound or a compositions refers to that amount of the compound or the composition that results in reduction or inhibition of symptoms or a prolongation of survival in a subject (i.e., a human patient). The results may require multiple doses of the compound or the composition.

“Treating” or “treatment” of a disease in a subject refers to 1) preventing the disease from occurring in a patient that is predisposed or does not yet display symptoms of the disease; 2) inhibiting the disease or arresting its development; or 3) ameliorating or causing regression of the disease. As used herein, “treatment” or “treating” is an approach for obtaining beneficial or desired results including clinical results. For the purposes of this disclosures, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delay or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a subject. Also encompassed by “treatment” is a reduction of pathological consequence of the disease or disorder. The methods of the disclosure contemplate any one or more of these aspects of treatment.

As used herein, the terms “individual(s)”, “subject(s)” and “patient(s)” mean any mammal. Examples include, but are not limited to, mice, rats, hamsters, guinea pigs, pigs, rabbits, cats, dogs, goats, sheep, cows, and humans. In some embodiments, the mammal is a human.

Although various features of the disclosure may be described in the context of a single embodiment, the features may also be provided separately or in any suitable combination. Conversely, although the disclosure may be described herein in the context of separate embodiments for clarity, the disclosure may also be implemented in a single embodiment.

### Compounds

In one aspect, provided herein is a compound of Formula (I):

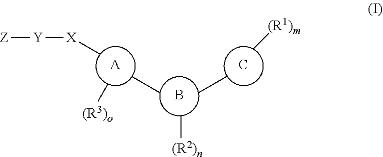

or a pharmaceutically acceptable salt thereof, wherein:
Ring A is a 5-membered heteroarylene containing 1-3 heteroatoms selected from N and S;
Ring B is phenylene or a 5- to 6-membered heteroarylene containing 1-3 heteroatoms selected from N and S;
Ring C is a fused bicyclic 9- to 10-membered heteroaryl or heterocyclyl containing 1-4 heteroatoms selected from N and O, a 5- to 6-membered heteroaryl containing 1-4 nitrogen atoms, or phenyl substituted by one R⁴;
each R¹ is independently —OH, halo, oxo, C₁-C₆ alkyl, C₁-C₆ haloalkyl, —CN, —N(H)C(O)R⁶, —N(H)SO₂R⁶, —N(H)SO₂(C₆H₅), —SO₂NR⁶R⁷, or —C(O)N(H)SO₂R⁶;
each R² is independently C₁-C₆ alkyl, C₁-C₆ haloalkyl, or halo;
each R³ is independently C₁-C₆ alkyl, C₁-C₆ haloalkyl, or C₃-C₆ cycloalkyl;
R⁴ is —OH, —OP(O)(OH)₂, —NO₂, —C(O)N(H)SO₂R⁵, 5- to 6-membered heteroaryl or heterocyclyl, or —C(O)N(H)-(5- to 6-membered heteroaryl or heterocyclyl), wherein the heteroaryl and heterocyclyl contain 1-4 heteroatoms selected from N, O, and S, and wherein the heterocyclyl is optionally substituted by 1-2 oxo groups;
m is 0-4;
n is 0-2;
o is 0-2;
R⁵ is phenyl or C₁-C₆ alkyl;
X is —CR⁶R⁷— or a bond;
Y is —O—, —N(H)—, or a bond;
each R⁶ and R⁷ is independently H or C₁-C₆ alkyl;
Z is Z¹ or Z²;
Z¹ is H, C₁-C₆ alkyl, —C(O)(C₁-C₆ alkyl), —CO₂(C₁-C₆ alkyl), —C(O)NR⁶R⁷, —C(O)NR⁶(C₁-C₆ alkylene)NR⁶R⁷, —CR⁶R⁷C(O)NR⁶R⁷, —CR⁶R⁷NR⁶R⁷, —C(O)(C₁-C₆ alkylene)NR⁶R⁷, —NR⁶C(O)(C₁-C₆ alkyl), —NR⁶R⁷, —(C₁-C₆ alkyl)-CO₂H, —(C₁-C₆ alkyl)-OH, or —C(NR⁶R⁷)═N—CN, wherein C₁-C₆ alkylene is optionally substituted by 1-6 halo, C₁-C₆ alkyl, or C₁-C₆ haloalkyl;
Z² is 5- to 6-membered heteroaryl, —C(O)(5- to 6-membered heteroaryl), —CH₂C(O)(5- to 6-membered heteroaryl), 4- to 6-membered heterocyclyl, —C(O)(4- to 6-membered heterocyclyl), or —CH₂C(O)(4- to 6-membered heterocyclyl), wherein the heteroaryl and heterocyclyl contain 1-3 heteroatoms selected from N and O, and wherein the heteroaryl and heterocyclyl are optionally substituted by 1-3 R⁸;
each R⁸ is independently halo, C₁-C₆ alkyl, C₁-C₆ haloalkyl, —NR⁶R⁷, —OH, oxo, —CO₂H, —O(C₁-C₆ alkyl), —CH₂—O(C₁-C₆ alkyl), or —(C₁-C₆ alkyl)-OH.

Provided herein is a compound of Formula (I-a):

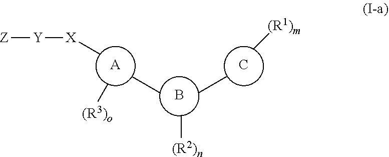

or a pharmaceutically acceptable salt thereof, wherein:
Ring A is thiazolylene;
Ring B is phenylene or a 5- to 6-membered heteroarylene containing 1-3 heteroatoms selected from N and S;
Ring C is a fused bicyclic 9- to 10-membered heteroaryl or heterocyclyl containing 1-4 heteroatoms selected from N and O, a 5- to 6-membered heteroaryl containing 1-4 nitrogen atoms, or phenyl substituted by one R⁴;
each R¹ is independently —OH, halo, oxo, C₁-C₆ alkyl, C₁-C₆ haloalkyl, —CN, —N(H)C(O)R⁶, —N(H)SO₂R⁶, —SO₂NR⁶R⁷, or —C(O)N(H)SO₂R⁶;
each R² is independently C₁-C₆ alkyl, C₁-C₆ haloalkyl, or halo;
each R³ is independently C₁-C₆ alkyl, C₁-C₆ haloalkyl, or C₃-C₆ cycloalkyl;
R⁴ is —OH, —OP(O)(OH)₂, —NO₂, —C(O)N(H)SO₂R⁵, 5- to 6-membered heteroaryl or heterocyclyl, or —C(O)N(H)-(5- to 6-membered heteroaryl or heterocyclyl), wherein the heteroaryl and heterocyclyl contain 1-4 heteroatoms selected from N, O, and S, and wherein the heterocyclyl is optionally substituted by 1-2 oxo groups;
m is 0-4;
n is 0-2;
o is 0-2;
R⁵ is phenyl or C₁-C₆ alkyl;
X is —CR⁶R⁷—;
Y is —O—, —N(H)—, or a bond;
each R⁶ and R⁷ is independently H or C₁-C₆ alkyl;
Z is Z²;
Z² is 5- to 6-membered heteroaryl, —C(O)(5- to 6-membered heteroaryl), —CH₂C(O)(5- to 6-membered heteroaryl), 4- to 6-membered heterocyclyl, —C(O)(4- to 6-membered heterocyclyl), or —CH₂C(O)(4- to 6-membered heterocyclyl), wherein the heteroaryl and heterocyclyl contain 1-3 heteroatoms selected from N and O, and wherein the heteroaryl and heterocyclyl are optionally substituted by 1-3 R⁸; and
each R⁸ is independently halo, C₁-C₆ alkyl, C₁-C₆ haloalkyl, —NR⁶R⁷, —OH, oxo, —CO₂H, —O(C₁-C₆ alkyl), —CH₂—O(C₁-C₆ alkyl), or —(C₁-C₆ alkyl)-OH.

Provided herein is a compound of Formula (I-a1):

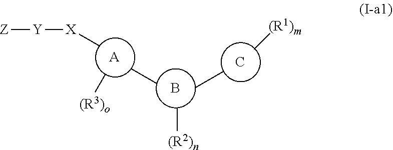

or a pharmaceutically acceptable salt thereof, wherein:
Ring A is thiazolylene;
Ring B is a 5- to 6-membered heteroarylene containing 1-3 heteroatoms selected from N and S;
Ring C is a fused bicyclic 9- to 10-membered heteroaryl or phenyl substituted by one R⁴;
each R¹ is independently —OH, halo, oxo, C₁-C₆ alkyl, C₁-C₆ haloalkyl, —CN, —N(H)C(O)R⁶, —N(H)SO₂R⁶, —SO₂NR⁶R⁷, or —C(O)N(H)SO₂R⁶;
each R² is independently C₁-C₆ alkyl, C₁-C₆ haloalkyl, or halo;
each R³ is independently C₁-C₆ alkyl, C₁-C₆ haloalkyl, or C₃-C₆ cycloalkyl;
R⁴ is —OH, —OP(O)(OH)₂, —NO₂, —C(O)N(H)SO₂R⁵, 5- to 6-membered heteroaryl or heterocyclyl, or —C(O)N(H)-(5- to 6-membered heteroaryl or heterocyclyl), wherein the heteroaryl and heterocyclyl contain 1-4 heteroatoms selected from N, O, and S, and wherein the heterocyclyl is optionally substituted by 1-2 oxo groups;
m is 0-4;
n is 0-2;
o is 0-2;
R⁵ is phenyl or C₁-C₆ alkyl;
X is —CR⁶R⁷—;
Y is —O—, —N(H)—, or a bond;
each R⁶ and R⁷ is independently H or C₁-C₆ alkyl;
Z is Z²;
Z² is 5- to 6-membered heteroaryl, —C(O)(5- to 6-membered heteroaryl), —CH₂C(O)(5- to 6-membered heteroaryl), 4- to 6-membered heterocyclyl, —C(O)(4- to 6-membered heterocyclyl), or —CH₂C(O)(4- to 6-membered heterocyclyl), wherein the heteroaryl and heterocyclyl contain 1-3 heteroatoms selected from N and O, and wherein the heteroaryl and heterocyclyl are optionally substituted by 1-3 R⁸; and
each R⁸ is independently halo, C₁-C₆ alkyl, C₁-C₆ haloalkyl, —NR⁶R⁷, —OH, oxo, —CO₂H, —O(C₁-C₆ alkyl), —CH₂—O(C₁-C₆ alkyl), or —(C₁-C₆ alkyl)-OH.

In some embodiments, Ring A is a 5-membered heteroarylene containing 1-3 heteroatoms selected from N and S. In some embodiments, Ring A is a 5-membered heteroarylene containing 1-2 heteroatoms selected from N and S. In some embodiments, Ring A is a 5-membered heteroarylene containing 1 heteroatom selected from N and S. In some embodiments, Ring A is a 5-membered heteroarylene containing 2 heteroatoms selected from N and S. In some embodiments, Ring A is a 5-membered heteroarylene containing one nitrogen atoms and one sulfur atom. In some embodiments, Ring A is thiazolylene.

In some embodiments, each R³ is independently C₁-C₆ alkyl, C₁-C₆ haloalkyl, or C₃-C₆ cycloalkyl. In some embodiments, each R³ is independently C₁-C₃ alkyl or C₃-C₆ cycloalkyl. In some embodiments, R³ is methyl, ethyl, or propyl. In some embodiments, R³ is methyl. In some embodiments, R³ is cyclopropyl, cyclobutyl, cyclopentyl, or cyclohexyl. In some embodiments, R³ is cyclopropyl. In some embodiments, each R³ is independently —CH₃ or cyclopropyl.

In some embodiments, o is 0-2. In some embodiments, o is 1-2. In some embodiments, o is 0 or 1. In some embodiments, o is 0. In some embodiments, o is 1. In some embodiments, o is 2.

In some embodiments, Ring B is phenylene or a 5- to 6-membered heteroarylene containing 1-3 heteroatoms selected from N and S. In some embodiments, Ring B is phenylene. In some embodiments, Ring B is a 5- to 6-membered heteroarylene containing 1-3 heteroatoms selected from N and S. In some embodiments, Ring B is a 5- to 6-membered heteroarylene containing 1-2 heteroatoms selected from N and S. In some embodiments, Ring B is phenylene, pyridinylene, thiazolylene, pyrazolylene, pyrimidinylene, or thienylene.

In some embodiments, each R² is independently C₁-C₆ alkyl, C₁-C₆ haloalkyl, or halo. In some embodiments, each R² is independently C₁-C₃ alkyl or halo. In some embodiments, R² is methyl, ethyl, or propyl. In some embodiments, R² is methyl. In some embodiments, R² is F, Cl, or Br.

In some embodiments, n is 0-2. In some embodiments, n is 0 or 1. In some embodiments, n is 0. In some embodiments, n is 1. In some embodiments, n is 2.

In some embodiments, Ring C is a fused bicyclic 9- to 10-membered heteroaryl or heterocyclyl containing 1-4 heteroatoms selected from N and O, a 5- to 6-membered heteroaryl containing 1-4 nitrogen atoms, or phenyl substituted by one R⁴. In some embodiments, Ring C is a fused bicyclic 9-membered heteroaryl or heterocyclyl containing 1-4 heteroatoms selected from N and O, a 5- to 6-membered heteroaryl containing 1-4 nitrogen atoms, or phenyl substituted by one R⁴. In some embodiments, Ring C is a fused bicyclic 9-membered heteroaryl or heterocyclyl containing 1-4 heteroatoms selected from N and O. In some embodiments, Ring C is a fused bicyclic 9-membered heteroaryl containing 1-4 heteroatoms selected from N and O. In some embodiments, Ring C is a fused bicyclic 9-membered heterocyclyl containing 1-4 heteroatoms selected from N and O. In some embodiments, one ring of the fused bicyclic heteroaryl or heterocyclyl is substituted by 0-4 R¹ groups. In some embodiments, both rings of the fused bicyclic heteroaryl or heterocyclyl are together substituted by 0-4 R¹ groups. In some embodiments, Ring C is a 5- to 6-membered heteroaryl containing 1-4 nitrogen atoms. In some embodiments, Ring C is a 5-membered heteroaryl containing 1-4 nitrogen atoms. In some embodiments, Ring C is a 6-membered heteroaryl containing 1-4 nitrogen atoms. In some embodiments, Ring C is phenyl substituted by one R⁴.

In some embodiments, R⁴ is —OH, —OP(O)(OH)₂, —NO₂, —C(O)N(H)SO₂R⁵, 5- to 6-membered heteroaryl or heterocyclyl, or —C(O)N(H)-(5- to 6-membered heteroaryl or heterocyclyl), wherein the heteroaryl and heterocyclyl contain 1-4 heteroatoms selected from N, O, and S, and wherein the heterocyclyl is optionally substituted by 1-2 oxo groups. In some embodiments, R⁴ is —OH. In some embodiments, R⁴ is —OP(O)(OH)₂. In some embodiments, R⁴ is —NO₂. In some embodiments, R⁴ is —C(O)N(H)SO₂R⁵. In some embodiments, R⁴ is 5- to 6-membered heteroaryl or heterocyclyl, wherein the heteroaryl and heterocyclyl contain 1-4 heteroatoms selected from N, O, and S, and wherein the heterocyclyl is optionally substituted by 1-2 oxo groups. In some embodiments, R⁴ is 5- to 6-membered heteroaryl containing 1-4 heteroatoms selected from N, O, and S. In some embodiments, R⁴ is 5- to 6-membered heterocyclyl containing 1-4 heteroatoms selected from N, O, and S, and optionally substituted by 1-2 oxo groups. In some embodiments, R⁴ is —C(O)N(H)-(5- to 6-membered heteroaryl or heterocyclyl), wherein the heteroaryl and heterocyclyl contain 1-4 heteroatoms selected from N, O, and S, and wherein the heterocyclyl is optionally substituted by 1-2 oxo groups. In some embodiments, R⁴ is —C(O)N(H)-(5- to 6-membered heteroaryl), wherein the heteroaryl contains 1-4 heteroatoms selected from N, O, and S. In some embodiments, R⁴ is —C(O)N(H)-(5- to 6-membered heterocyclyl), wherein the heterocyclyl contains 1-4 heteroatoms selected from N, O, and S, and is optionally substituted by 1-2 oxo groups. In some embodiments, R⁴ is —OH,
—OP(O)(OH)₂, —NO₂, —C(O)N(H)SO₂R⁵, 5- to 6-membered heteroaryl or heterocyclyl, or
—C(O)N(H)-(5- to 6-membered heteroaryl or heterocyclyl), wherein the heteroaryl and heterocyclyl contain 1-4 heteroatoms selected from N, O, and S, and wherein the heterocyclyl is optionally substituted by 1-2 oxo groups. In any of these variations, R⁵ is phenyl or C₁-C₆ alkyl. In some embodiments, R⁵ is phenyl or C₁-C₃ alkyl.

In some embodiments, Ring C—(R¹)ₘ is selected from

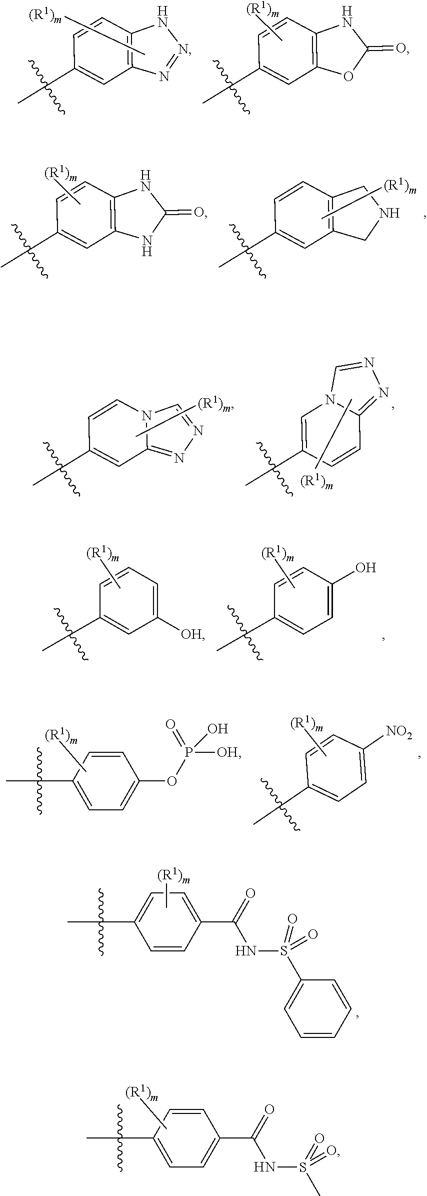

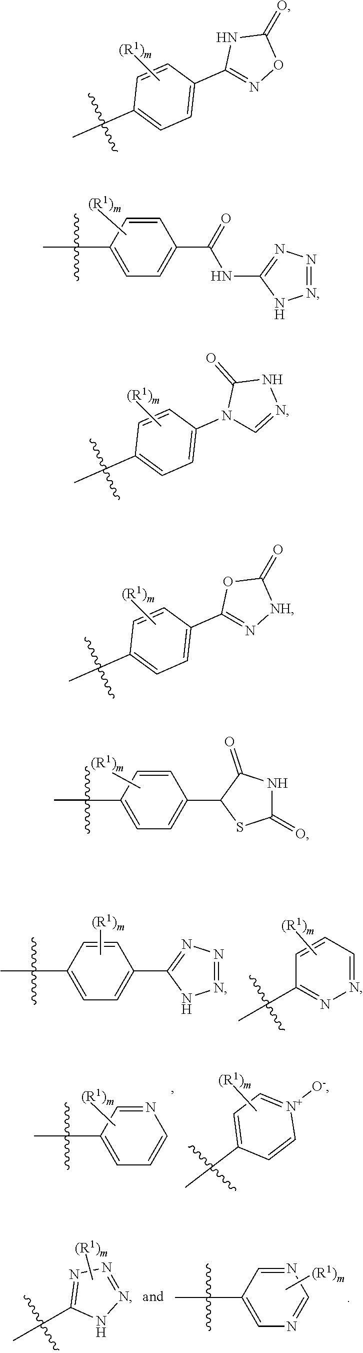

As shown above, certain nitrogen atoms of the heteroaryl and heterocyclyl rings are represented as —NH— in order to complete the valency. However, it is understood that the —NH— group may also be substituted by R¹ (—NR¹—). It is also understood that when —(R¹)ₘ is drawn through a fused bicyclic heteroaryl or heterocyclyl, then one or both of the fused rings may be substituted by R¹ groups. In some embodiments, only one ring of the fused bicyclic ring is substituted by R¹ groups. In some embodiments, both rings of the fused bicyclic ring are substituted by R¹ groups, such that the total number of R¹ groups substituting the bicyclic ring is 0-4.

In some embodiments, each R¹ is independently —OH, halo, oxo, C₁-C₆ alkyl, C₁-C₆ haloalkyl, —CN, —N(H)C(O)R⁶, —N(H)SO₂R⁶, —N(H)SO₂(C₆H₅), —SO₂NR⁶R⁷, or —C(O)N(H)SO₂R⁶. In some embodiments, each R¹ is independently —OH, halo, oxo, C₁-C₆ alkyl, C₁-C₆ haloalkyl, —CN, —N(H)C(O)R⁶, —N(H)SO₂R⁶, —SO₂NR⁶R⁷, or —C(O)N(H)SO₂R⁶. In some embodiments, each R¹ is independently —OH, halo, oxo, C₁-C₃ alkyl, C₁-C₃ haloalkyl, —CN, —N(H)C(O)R⁶, —N(H)SO₂R⁶, —SO₂NR⁶R⁷, or —C(O)N(H)SO₂R⁶.

In some embodiments, R¹ is —OH. In some embodiments, R¹ is halo. In some embodiments, R¹ is F, Cl, or Br. In some embodiments, R¹ is oxo. In some embodiments, R¹ is C₁-C₆ alkyl. In some embodiments, R¹ is C₁-C₃ alkyl. In some embodiments, R¹ is methyl, ethyl, or propyl. In some embodiments, R¹ is C₁-C₆ haloalkyl. In some embodiments, R¹ is C₁-C₃ haloalkyl. In some embodiments, R¹ is —CN. In some embodiments, R¹ is —N(H)C(O)R⁶. In some embodiments, R¹ is —N(H)SO₂R⁶. In some embodiments, R¹ is —SO₂NR⁶R⁷. In some embodiments, R¹ is —C(O)N(H)SO₂R⁶. In some embodiments, each R¹ is independently —OH, halo, oxo, C₁-C₃ alkyl, C₁-C₃ haloalkyl, —CN, —N(H)C(O)R⁶, —N(H)SO₂R⁶, —SO₂NR⁶R⁷, or —C(O)N(H)SO₂R⁶. In any of these variations, each R⁶ and R⁷ is independently H or C₁-C₆ alkyl. In some embodiments, each R⁶ and R⁷ is independently H or C₁-C₃ alkyl. In some embodiments, each R¹ is independently —OH, F, oxo, —CH₃, —CN, —N(H)C(O)CH₃, —N(H)SO₂(C₆H₅), —N(H)SO₂CH₃, —SO₂NH₂, or —C(O)N(H)SO₂CH₃.

In some embodiments, m is 0-4. In some embodiments, m is 0-3. In some embodiments, m is 0. In some embodiments, m is 1-3. In some embodiments, m is 1. In some embodiments, m is 2. In some embodiments, m is 3. In some embodiments, m is 4.

In some embodiments, R⁵ is phenyl or C₁-C₆ alkyl. In some embodiments, R⁵ is phenyl or C₁-C₃ alkyl. In some embodiments, R⁵ is phenyl. In some embodiments, R⁵ is C₁-C₆ alkyl. In some embodiments, R⁵ is C₁-C₃ alkyl. In some embodiments, R⁵ is methyl, ethyl, or propyl. In some embodiments, R⁵ is methyl. In some embodiments, R⁵ is phenyl or methyl.

In some embodiments, X is —CR⁶R⁷— or a bond. In some embodiments, X is —CR⁶R⁷—. In some variations, R⁶ and R⁷ are independently H or C₁-C₆ alkyl. In some variations, R⁶ and R⁷ are each H, and X is —CH₂—. In some embodiments, X is a bond.

In some embodiments, Y is —O—, —N(H)—, or a bond. In some embodiments, Y is —O—. In some embodiments, Y is —N(H)—. In some embodiments, Y is a bond.

In some embodiments, each R⁶ and R⁷ is independently H or C₁-C₆ alkyl. In some embodiments, each R⁶ and R⁷ is independently H or C₁-C₃ alkyl. In some embodiments, R⁶ and R⁷ are each H. In some embodiments, R⁶ is H. In some embodiments, R⁷ is H. In some embodiments, R⁶ is C₁-C₆ alkyl. In some embodiments, R⁷ is C₁-C₆ alkyl. In some embodiments, R⁶ is H and R⁷ is C₁-C₆ alkyl. In some embodiments, R⁶ is H and R⁷ is C₁-C₃ alkyl. In some embodiments, R⁶ and R⁷ are each independently C₁-C₆ alkyl. In some embodiments, R⁶ and R⁷ are each independently C₁-C₃ alkyl. In some embodiments, R⁶ and R⁷ are each independently methyl, ethyl, or propyl. In some embodiments, R⁶ and R⁷ are each methyl.

In some embodiments, Z is Z¹.

In some embodiments, Z¹ is H, C₁-C₆ alkyl, —C(O)(C₁-C₆ alkyl), —CO₂(C₁-C₆ alkyl), —C(O)NR⁶R⁷, —C(O)NR⁶(C₁-C₆ alkylene)NR⁶R⁷, —CR⁶R⁷C(O)NR⁶R⁷, —CR⁶R⁷NR⁶R⁷, —C(O)(C₁-C₆ alkylene)NR⁶R⁷, —NR⁶C(O)(C₁-C₆ alkyl), —NR⁶R⁷, —(C₁-C₆ alkyl)-CO₂H, —(C₁-C₆ alkyl)-OH, or —C(NR⁶R⁷)═N—CN. In any of these variations, C₁-C₆ alkylene is optionally substituted by 1-6 halo, C₁-C₆ alkyl, or C₁-C₆haloalkyl. In some embodiments, Z¹ is H, C₁-C₃ alkyl, —C(O)(C₁-C₃ alkyl), —CO₂(C₁-C₃ alkyl), —C(O)NR⁶R⁷, —C(O)NR⁶(C₁-C₃ alkylene)NR⁶R⁷, —CR⁶R⁷C(O)NR⁶R⁷, —CR⁶R⁷NR⁶R⁷, —C(O)(C₁-C₃ alkylene)NR⁶R⁷, —NR⁶C(O)(C₁-C₃ alkyl), —NR⁶R⁷, —(C₁-C₃ alkyl)-CO₂H, or —(C₁-C₃ alkyl)-OH, or —C(NR⁶R⁷)═N—CN. In any of these variations, C₁-C₃ alkylene is optionally substituted by 1-2 halo, C₁-C₃ alkyl, or C₁-C₃ haloalkyl. In some embodiments, each R⁶ and R⁷ is independently H or C₁-C₃ alkyl. In some embodiments, Z¹ is H, —CO₂CH₃, —CO₂C(CH₃)₃, —C(O)CH₃, —C(O)C(CH₃)₃, —C(N(CH₃)₂)═N—CN, —C(O)N(CH₃)₂, —CH₃, —CH₂CH₃, —CH₂C(CH₃)₃, —CH₂CO₂H, —CH₂C(CH₃)₂OH, —C(O)N(CH₃)CH₂CH₂N(CH₃)₂, —CH₂C(O)N(CH₃)₂, —CH(CH₃)C(O)N(CH₃)₂, —N(CH₃)₂, —N(CH₃)C(O)CH₃, or —C(O)CH(CH₃)CH₂N(CH₃)₂.

In some embodiments, if X is a bond and Y is a bond; and Z¹ is not hydrogen.

In some embodiments, Z is Z².

In some embodiments, Z² is 5- to 6-membered heteroaryl, —C(O)(5- to 6-membered heteroaryl), —CH₂C(O)(5- to 6-membered heteroaryl), 4- to 6-membered heterocyclyl, —C(O)(4- to 6-membered heterocyclyl), or —CH₂C(O)(4- to 6-membered heterocyclyl), wherein the heteroaryl and heterocyclyl contain 1-3 heteroatoms selected from N and O, and wherein the heteroaryl and heterocyclyl are optionally substituted by 1-3 R⁸. In some embodiments, Z² is 5- to 6-membered heteroaryl, —C(O)(5- to 6-membered heteroaryl), —CH₂C(O)(5- to 6-membered heteroaryl), 4- to 6-membered heterocyclyl, —C(O)(4- to 6-membered heterocyclyl), or —CH₂C(O)(4- to 6-membered heterocyclyl), wherein the heteroaryl and heterocyclyl contain 1-2 heteroatoms selected from N and O, and wherein the heteroaryl and heterocyclyl are optionally substituted by 1-3 R⁸. In some embodiments, Z² is pyrimidinyl, pyridazinyl, pyrrolidinyl, piperidinyl, pyridazinyl, —C(O)(tetrahydropyranyl), —C(O)(pyrrolidinyl), —C(O)(pyridazinyl), —C(O)(piperidinyl), —C(O)(azetidinyl), —C(O)(pyrazolyl), —C(O)(piperazinyl), —CH₂C(O)(pyrrolidinyl), —CH₂C(O)(piperidinyl), or —CH₂C(O)(piperazinyl), wherein the heteroaryl and heterocyclyl are optionally substituted by 1-3 R⁸.

In some embodiments, each R⁸ is independently halo, C₁-C₆ alkyl, C₁-C₆ haloalkyl, —NR⁶R⁷, —OH, oxo, —CO₂H, —O(C₁-C₆ alkyl), —CH₂—O(C₁-C₆ alkyl), or —(C₁-C₆ alkyl)-OH. In some embodiments, each R⁸ is independently halo, C₁-C₃ alkyl, C₁-C₃ haloalkyl, —NR⁶R⁷, —OH, oxo, —CO₂H, —O(C₁-C₃ alkyl), —CH₂—O(C₁-C₃ alkyl), or —(C₁-C₃ alkyl)-OH. In some variations, each R⁶ and R⁷ is independently H or C₁-C₃ alkyl. In some embodiments, each R⁸ is independently —CH₃, —CH₂CH₃, —NH₂, —OH, oxo, —N(CH₃)₂, —OCH₃, —CH₂OCH₃, —CF₃,

—CH₂CH₂OH, —CO₂H, or Cl.

In some embodiments, Z—Y—X— is

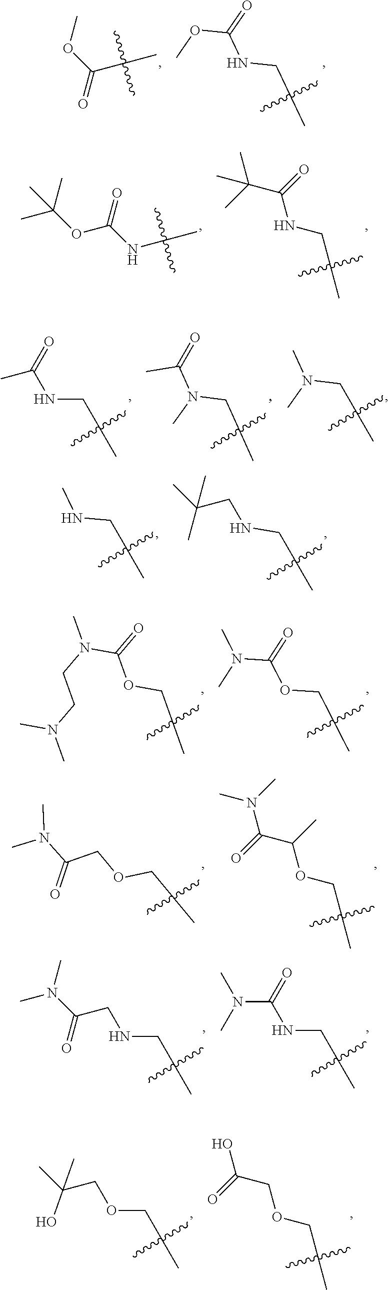

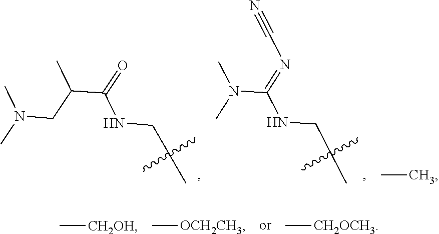

In some embodiments, Z—Y—X— is

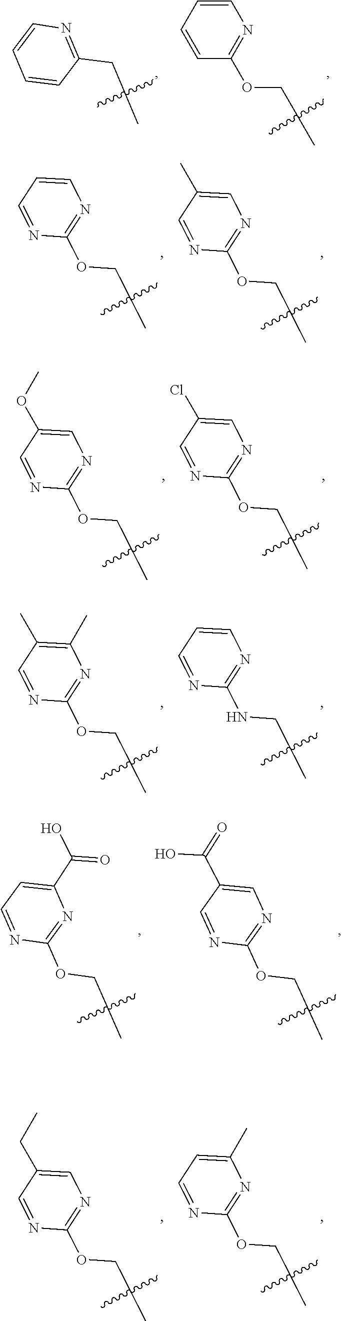

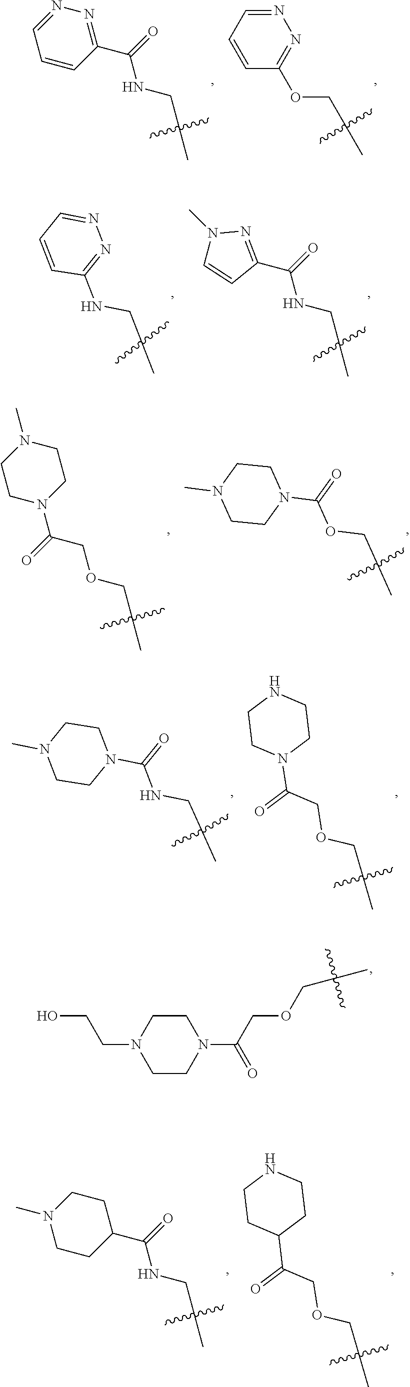

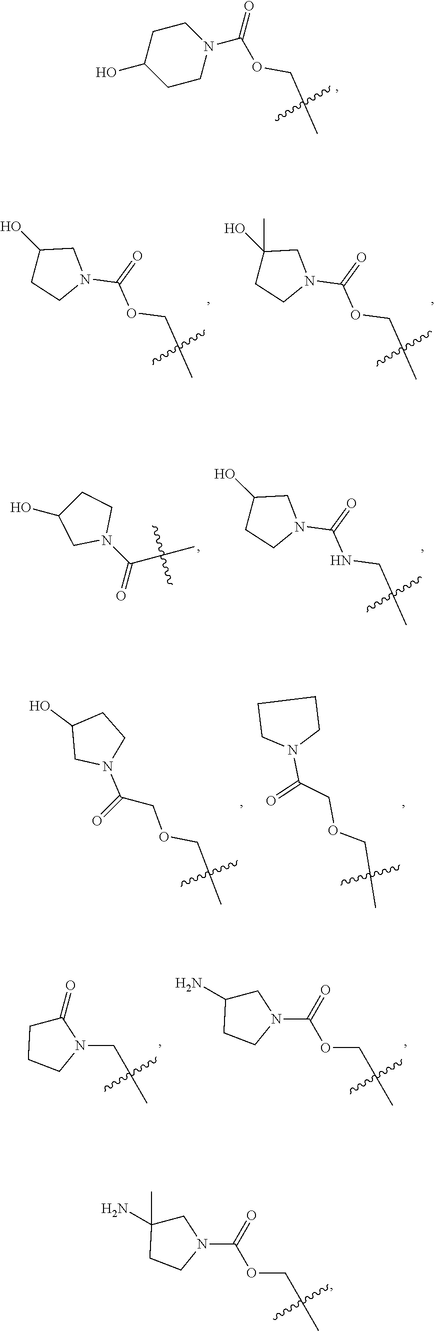

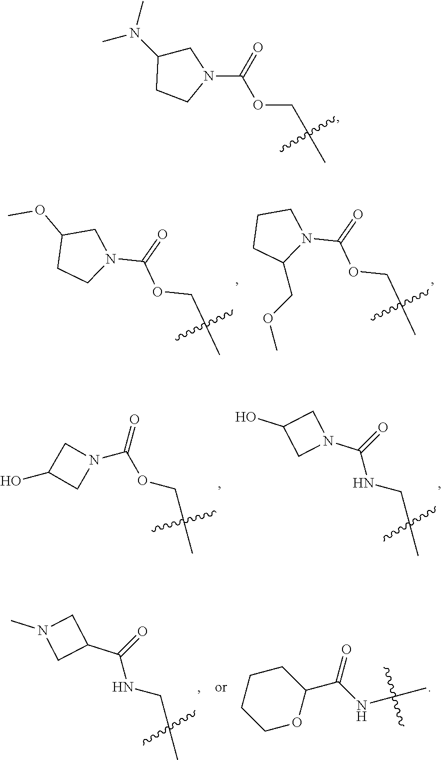

In some embodiments, the compound of Formula (I) is a compound of Formula (II), (III), (IV), (V), or (VI):

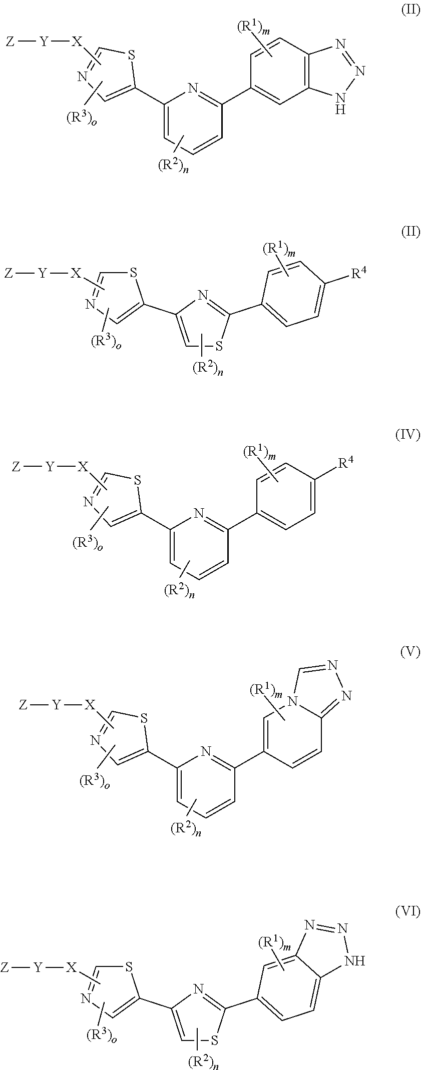

wherein R¹, m, R², n, R³, o, X, Y, and Z are as described for Formula (I).

In some embodiments, the compound of Formula (I) is a compound of Formula (II), wherein R¹, m, R², n, R³, o, X, Y, and Z are as described for Formula (I). In some embodiments, the compound of Formula (I) is a compound of Formula (III), wherein R¹, m, R², n, R³, o, X, Y, and Z are as described for Formula (I). In some embodiments, the compound of Formula (I) is a compound of Formula (IV), wherein R¹, m, R², n, R³, o, X, Y, and Z are as described for Formula (I). In some embodiments, the compound of Formula (I) is a compound of Formula (V), wherein R¹, m, R², n, R³, o, X, Y, and Z are as described for Formula (I). In some embodiments, the compound of Formula (VI) is a compound of Formula (II), wherein R¹, m, R², n, R³, o, X, Y, and Z are as described for Formula (I).

In some embodiments, the compound is of Formula (II)-(VI) and X is —CH₂—. In some embodiments, the compound is of Formula (II)-(VI) and Y is —O— or —N(H)—. In some embodiments, the compound is of Formula (II)-(VI) and R¹ is halo (such as fluoro), —CN, or —OH, and m is 1, 2, or 3. In some embodiments, the compound is of Formula (II)-(VI) and o is 0. In some embodiments, the compound is of Formula (II)-(VI) and o is 1. In some embodiments, the compound is of Formula (II)-(VI) and n is 0. In some embodiments, the compound is of Formula (II)-(VI) and R³ is C₁-C₆ alkyl (such as methyl), and o is 1. In some embodiments, the compound is of Formula (II)-(VI) and Z is 6-membered heteroaryl (such as pyridazinyl, pyrimidinyl, or pyridinyl) optionally substituted by C₁-C₆ alkyl (such as methyl). In some embodiments, the compound is of Formula (II)-(VI) and Z is —CO₂(C₁-C₆ alkyl) (such as —CO₂CH₃) or —(C₁-C₆ alkyl)-OH (such as —CH₂C(CH₃)₂OH). In some embodiments, the compound is of Formula (II)-(VI) and R⁴ is —OH or 6-membered heteroaryl or heterocyclyl (such as tetrazolyl).

In some embodiments, provided herein is compound of Formula (I) is a compound of Formula (II), wherein X is —CH₂—; Y is —O— or —N(H)—; Z is 6-membered heteroaryl optionally substituted by C₁-C₆ alkyl, —CO₂(C₁-C₆ alkyl), or —(C₁-C₆ alkyl)-OH; and R¹, m, R², n, R³, and o are as described for Formula (I). In some embodiments, provided herein is compound of Formula (I) is a compound of Formula (III), wherein X is —CH₂—; Y is —O— or —N(H)—; Z is 6-membered heteroaryl optionally substituted by C₁-C₆ alkyl, —CO₂(C₁-C₆ alkyl), or —(C₁-C₆ alkyl)-OH; and R¹, m, R², n, R³, and o are as described for Formula (I). In some embodiments, provided herein is compound of Formula (I) is a compound of Formula (IV), wherein X is —CH₂—; Y is —O— or —N(H)—; Z is 6-membered heteroaryl optionally substituted by C₁-C₆ alkyl, —CO₂(C₁-C₆ alkyl), or —(C₁-C₆ alkyl)-OH; and R¹, m, R², n, R³, and o are as described for Formula (I). In some embodiments, provided herein is compound of Formula (I) is a compound of Formula (V), wherein X is —CH₂—; Y is —O— or —N(H)—; Z is 6-membered heteroaryl optionally substituted by C₁-C₆ alkyl, —CO₂(C₁-C₆ alkyl), or —(C₁-C₆ alkyl)-OH; and R¹, m, R², n, R³, and o are as described for Formula (I). In some embodiments, provided herein is compound of Formula (I) is a compound of Formula (VI), wherein X is —CH₂—; Y is —O— or —N(H)—; Z is 6-membered heteroaryl optionally substituted by C₁-C₆ alkyl, —CO₂(C₁-C₆ alkyl), or —(C₁-C₆ alkyl)-OH; and R¹, m, R², n, R³, and o are as described for Formula (I).

In some embodiments, provided herein is compound of Formula (I) is a compound of Formula (II), wherein X is —CH₂—; Y is —O— or —N(H)—; Z is 6-membered heteroaryl optionally substituted by C₁-C₆ alkyl; and R¹, m, R², n, R³, and o are as described for Formula (I). In some embodiments, provided herein is compound of Formula (I) is a compound of Formula (III), wherein X is —CH₂—; Y is —O— or —N(H)—; Z is 6-membered heteroaryl optionally substituted by C₁-C₆ alkyl; and R¹, m, R², n, R³, and o are as described for Formula (I). In some embodiments, provided herein is compound of Formula (I) is a compound of Formula (IV), wherein X is —CH₂—; Y is —O— or —N(H)—; Z is 6-membered heteroaryl optionally substituted by C₁-C₆ alkyl; and R¹, m, R², n, R³, and o are as described for Formula (I). In some embodiments, provided herein is compound of Formula (I) is a compound of Formula (V), wherein X is —CH₂—; Y is —O— or —N(H)—; Z is 6-membered heteroaryl optionally substituted by C₁-C₆ alkyl; and R¹, m, R², n, R³, and o are as described for Formula (I). In some embodiments, provided herein is compound of Formula (I) is a compound of Formula (VI), wherein X is —CH₂—; Y is —O— or —N(H)—; Z is 6-membered heteroaryl optionally substituted by C₁-C₆ alkyl; and R¹, m, R², n, R³, and o are as described for Formula (I).

In some embodiments, provided herein is compound of Formula (I) is a compound of Formula (II), wherein X is —CH₂—; Y is —O— or —N(H)—; Z is —CO₂(C₁-C₆ alkyl) or —(C₁-C₆ alkyl)-OH; and R¹, m, R², n, R³, and o are as described for Formula (I). In some embodiments, provided herein is compound of Formula (I) is a compound of Formula (III), wherein X is —CH₂—; Y is —O— or —N(H)—; Z is —CO₂(C₁-C₆ alkyl) or —(C₁-C₆ alkyl)-OH; and R¹, m, R², n, R³, and o are as described for Formula (I). In some embodiments, provided herein is compound of Formula (I) is a compound of Formula (IV), wherein X is —CH₂—; Y is —O— or —N(H)—; Z is —CO₂(C₁-C₆ alkyl) or —(C₁-C₆ alkyl)-OH; and R¹, m, R², n, R³, and o are as described for Formula (I). In some embodiments, provided herein is compound of Formula (I) is a compound of Formula (V), wherein X is —CH₂—; Y is —O— or —N(H)—; Z is —CO₂(C₁-C₆ alkyl) or —(C₁-C₆ alkyl)-OH; and R¹, m, R², n, R³, and o are as described for Formula (I). In some embodiments, provided herein is compound of Formula (VI) is a compound of Formula (II), wherein X is —CH₂—; Y is —O— or —N(H)—; Z is —CO₂(C₁-C₆ alkyl) or —(C₁-C₆ alkyl)-OH; and R¹, m, R², n, R³, and o are as described for Formula (I).

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to L of Formula (I) may be combined with every description, variation, embodiment, or aspect of Ring A, Ring B, Ring C, R¹, R², R³, m, n, o, R⁴, R⁵, R⁶, R⁷, X, Y, Z, Z¹, Z², and R⁸ the same as if each and every combination were specifically and individually listed. It is also understood that all descriptions, variations, embodiments, or aspects of Formula (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (I), where applicable, apply equally to any of the formulae as detailed herein, such as Formulae (II)-(VI), and are equally described, the same as if each and every description, variation, embodiment, or aspect were separately and individually listed for all formulae.

In some embodiments, provided is a compound selected from the compounds in Table 1 or a pharmaceutically acceptable salt thereof. In some embodiments, provided is a compound selected from the compounds in Table 2 or a pharmaceutically acceptable salt thereof. Although certain compounds described in the present disclosure, including in Table 1 and Table 2, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1 and Table 2, are herein described.

TABLE 1

| Example | Structure |
|---|---|
| 7 | 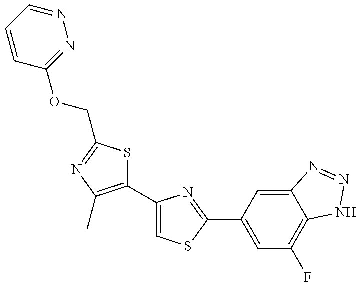 [SMILES: Cc1nc(COc2cccnn2)sc1-c1csc(-c2cc(F)c3[nH]nnc3c2)n1] |
| 8 | 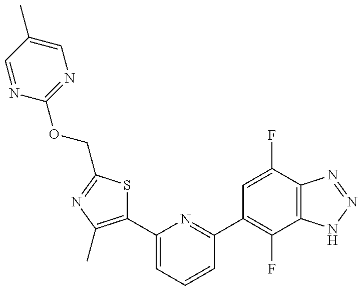 [SMILES: Cc1cnc(OCc2nc(C)c(-c3cccc(-c4cc(F)c5nn[nH]c5c4F)n3)s2)nc1] |
| 9 | 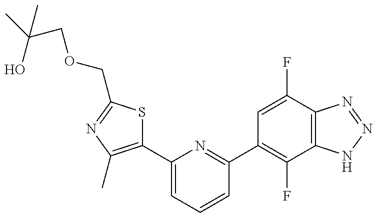 [SMILES: Cc1nc(COCC(C)(C)O)sc1-c1cccc(-c2cc(F)c3nn[nH]c3c2F)n1] |
| 10 | 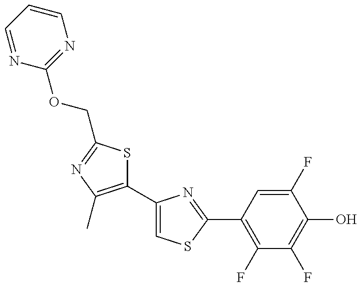 [SMILES: Cc1nc(COc2ncccn2)sc1-c1csc(-c2cc(F)c(O)c(F)c2F)n1] |
| 11 | 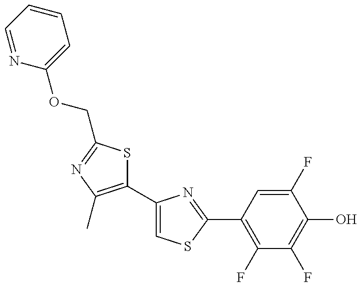 [SMILES: Cc1nc(COc2ccccn2)sc1-c1csc(-c2cc(F)c(O)c(F)c2F)n1] |
| 12 | 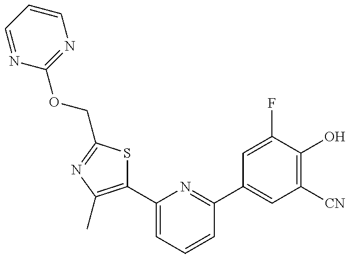 [SMILES: Cc1nc(COc2ncccn2)sc1-c1cccc(-c2cc(F)c(O)c(C#N)c2)n1] |
| 13 | 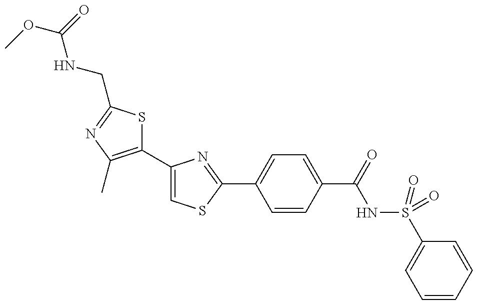 [SMILES: COC(=O)NCc1nc(C)c(-c2csc(-c3ccc(C(=O)NS(=O)(=O)c4ccccc4)cc3)n2)s1] |
| 14 | 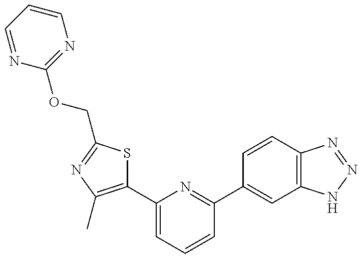 [SMILES: Cc1nc(COc2ncccn2)sc1-c1cccc(-c2ccc3nn[nH]c3c2)n1] |
| 15 | 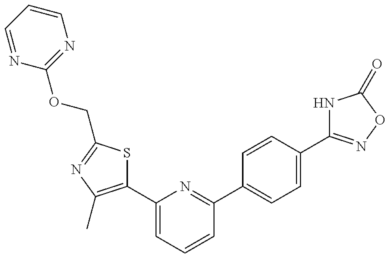 [SMILES: Cc1nc(COc2ncccn2)sc1-c1cccc(-c2ccc(-c3noc(=O)[nH]3)cc2)n1] |
| 16 | 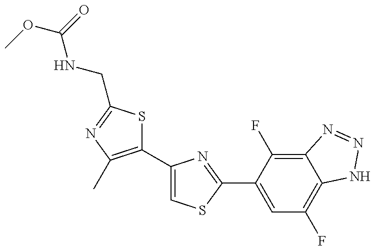 [SMILES: COC(=O)NCc1nc(C)c(-c2csc(-c3cc(F)c4[nH]nnc4c3F)n2)s1] |

TABLE 2

| Example | Structure |
|---|---|
| 17 | 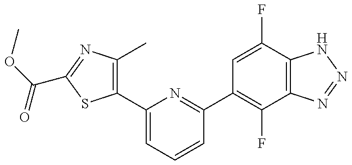 [SMILES: COC(=O)c1nc(C)c(-c2cccc(-c3cc(F)c4[nH]nnc4c3F)n2)s1] |
| 18 | 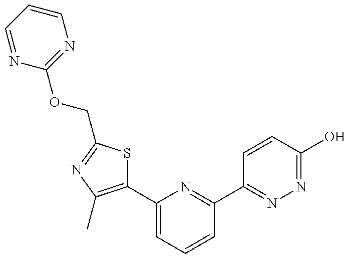 [SMILES: Cc1nc(COc2ncccn2)sc1-c1cccc(-c2ccc(O)nn2)n1] |
| 19 | 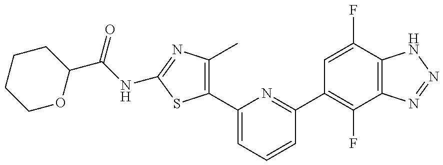 [SMILES: Cc1nc(NC(=O)C2CCCCO2)sc1-c1cccc(-c2cc(F)c3[nH]nnc3c2F)n1] |
| 20 | 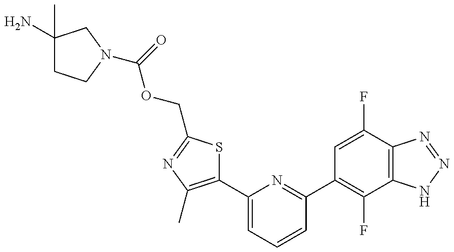 [SMILES: Cc1nc(COC(=O)N2CCC(C)(N)C2)sc1-c1cccc(-c2cc(F)c3nn[nH]c3c2F)n1] |
| 21 | 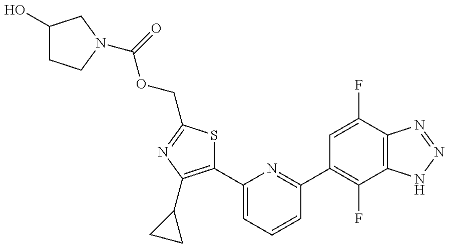 [SMILES: O=C(OCc1nc(C2CC2)c(-c2cccc(-c3cc(F)c4nn[nH]c4c3F)n2)s1)N1CCC(O)C1] |
| 22 | 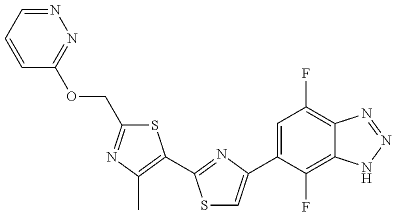 [SMILES: Cc1nc(COc2cccnn2)sc1-c1nc(-c2cc(F)c3nn[nH]c3c2F)cs1] |
| 23 | 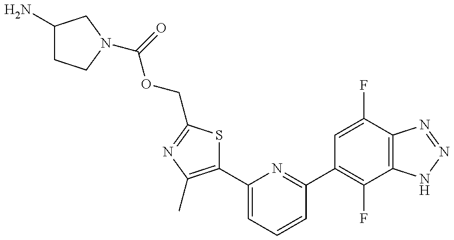 [SMILES: Cc1nc(COC(=O)N2CCC(N)C2)sc1-c1cccc(-c2cc(F)c3nn[nH]c3c2F)n1] |
| 24 | 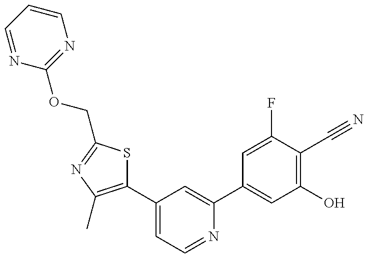 [SMILES: Cc1nc(COc2ncccn2)sc1-c1ccnc(-c2cc(O)c(C#N)c(F)c2)c1] |
| 25 | 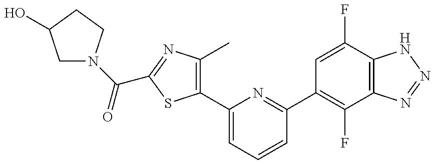 [SMILES: Cc1nc(C(=O)N2CCC(O)C2)sc1-c1cccc(-c2cc(F)c3[nH]nnc3c2F)n1] |
| 26 | 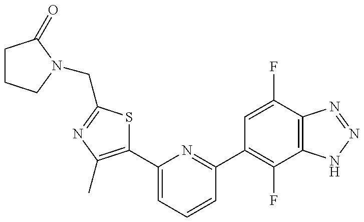 [SMILES: Cc1nc(CN2CCCC2=O)sc1-c1cccc(-c2cc(F)c3nn[nH]c3c2F)n1] |
| 27 | 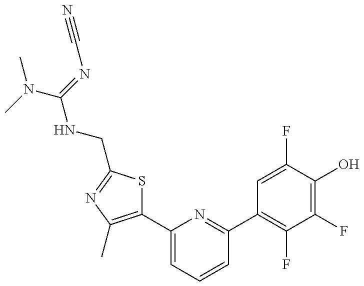 [SMILES: Cc1nc(CN/C(=N/C#N)N(C)C)sc1-c1cccc(-c2cc(F)c(O)c(F)c2F)n1] |
| 28 | 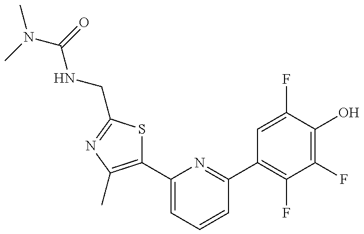 [SMILES: Cc1nc(CNC(=O)N(C)C)sc1-c1cccc(-c2cc(F)c(O)c(F)c2F)n1] |
| 29 | 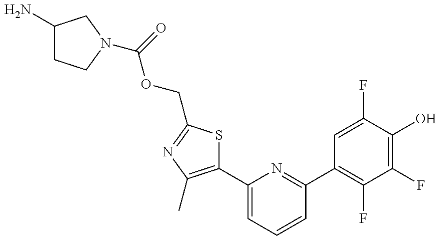 [SMILES: Cc1nc(COC(=O)N2CCC(N)C2)sc1-c1cccc(-c2cc(F)c(O)c(F)c2F)n1] |
| 30 | 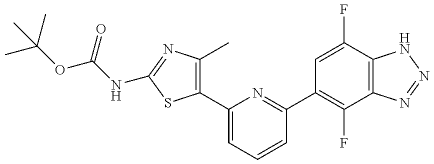 [SMILES: Cc1nc(NC(=O)OC(C)(C)C)sc1-c1cccc(-c2cc(F)c3[nH]nnc3c2F)n1] |
| 31 | 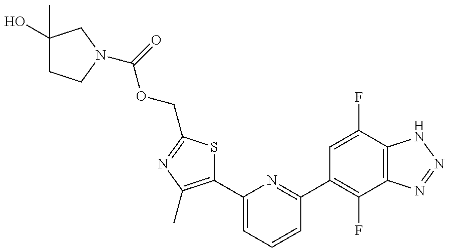 [SMILES: Cc1nc(COC(=O)N2CCC(C)(O)C2)sc1-c1cccc(-c2cc(F)c3[nH]nnc3c2F)n1] |
| 32 | 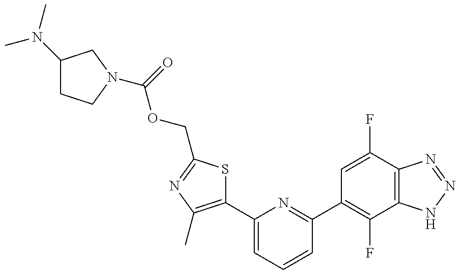 [SMILES: Cc1nc(COC(=O)N2CCC(N(C)C)C2)sc1-c1cccc(-c2cc(F)c3nn[nH]c3c2F)n1] |
| 33 | 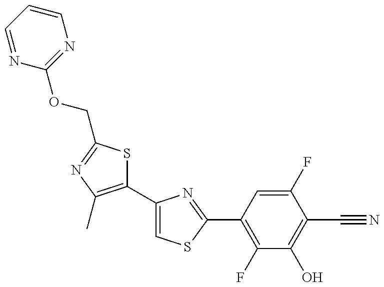 [SMILES: Cc1nc(COc2ncccn2)sc1-c1csc(-c2cc(F)c(C#N)c(O)c2F)n1] |
| 34 | 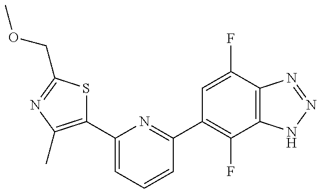 [SMILES: COCc1nc(C)c(-c2cccc(-c3cc(F)c4nn[nH]c4c3F)n2)s1] |
| 35 | 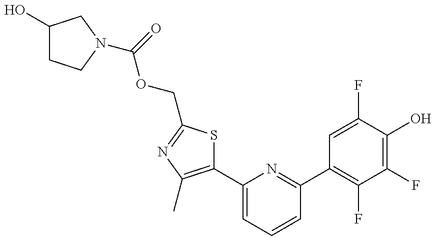 [SMILES: Cc1nc(COC(=O)N2CCC(O)C2)sc1-c1cccc(-c2cc(F)c(O)c(F)c2F)n1] |
| 36 | 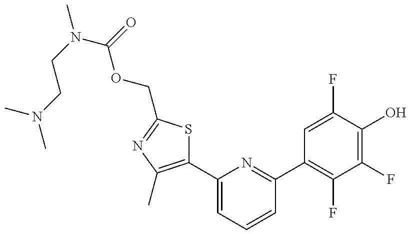 [SMILES: Cc1nc(COC(=O)N(C)CCN(C)C)sc1-c1cccc(-c2cc(F)c(O)c(F)c2F)n1] |
| 37 | 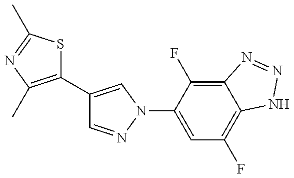 [SMILES: Cc1nc(C)c(-c2cnn(-c3cc(F)c4[nH]nnc4c3F)c2)s1] |
| 38 | 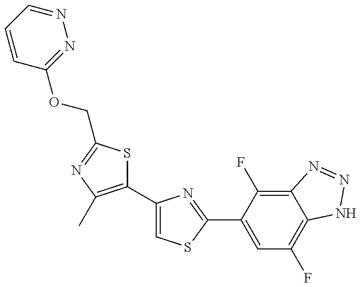 [SMILES: Cc1nc(COc2cccnn2)sc1-c1csc(-c2cc(F)c3[nH]nnc3c2F)n1] |
| 39 | 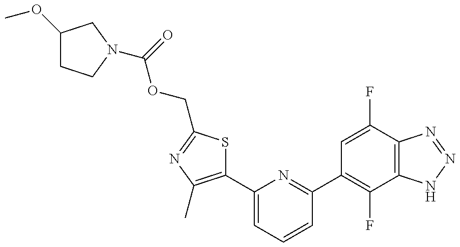 [SMILES: COC1CCN(C(=O)OCc2nc(C)c(-c3cccc(-c4cc(F)c5nn[nH]c5c4F)n3)s2)C1] |
| 40 | 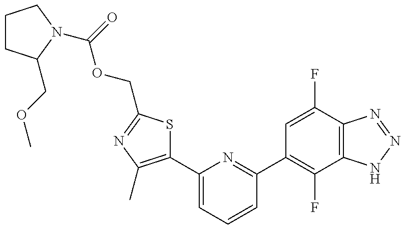 [SMILES: COCC1CCCN1C(=O)OCc1nc(C)c(-c2cccc(-c3cc(F)c4nn[nH]c4c3F)n2)s1] |
| 41 | 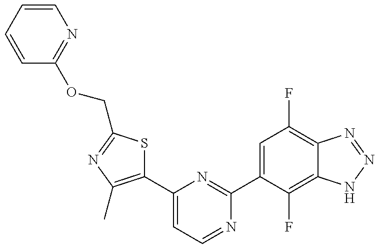 [SMILES: Cc1nc(COc2ccccn2)sc1-c1ccnc(-c2cc(F)c3nn[nH]c3c2F)n1] |
| 42 | 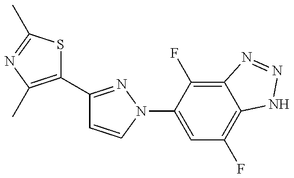 [SMILES: Cc1nc(C)c(-c2ccn(-c3cc(F)c4[nH]nnc4c3F)n2)s1] |
| 43 | 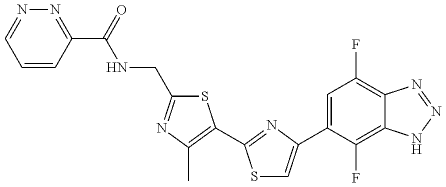 [SMILES: Cc1nc(CNC(=O)c2cccnn2)sc1-c1nc(-c2cc(F)c3nn[nH]c3c2F)cs1] |
| 44 | 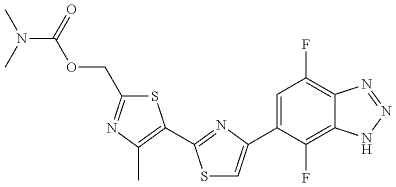 [SMILES: Cc1nc(COC(=O)N(C)C)sc1-c1nc(-c2cc(F)c3nn[nH]c3c2F)cs1] |
| 45 | 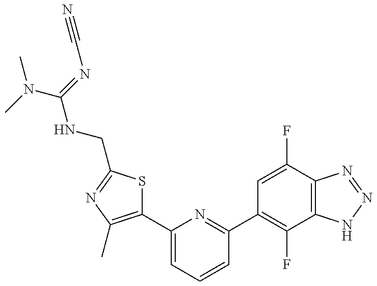 [SMILES: Cc1nc(CN/C(=N/C#N)N(C)C)sc1-c1cccc(-c2cc(F)c3nn[nH]c3c2F)n1] |
| 46 | 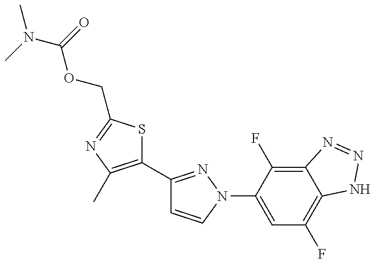 [SMILES: Cc1nc(COC(=O)N(C)C)sc1-c1ccn(-c2cc(F)c3[nH]nnc3c2F)n1] |
| 47 | 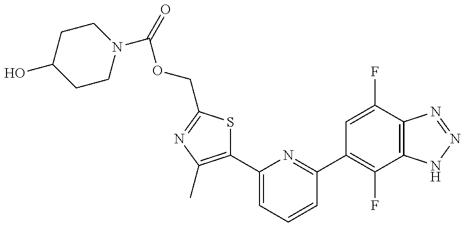 [SMILES: Cc1nc(COC(=O)N2CCC(O)CC2)sc1-c1cccc(-c2cc(F)c3nn[nH]c3c2F)n1] |
| 48 | 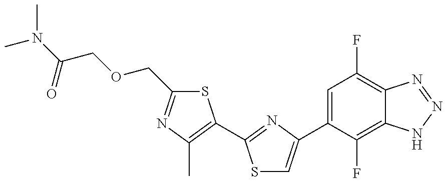 [SMILES: Cc1nc(COCC(=O)N(C)C)sc1-c1nc(-c2cc(F)c3nn[nH]c3c2F)cs1] |
| 49 | 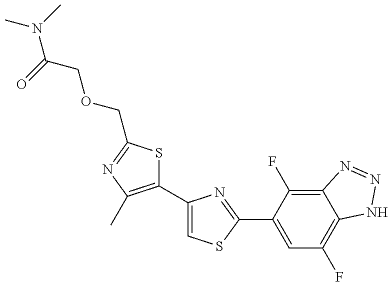 [SMILES: Cc1nc(COCC(=O)N(C)C)sc1-c1csc(-c2cc(F)c3[nH]nnc3c2F)n1] |
| 50 | 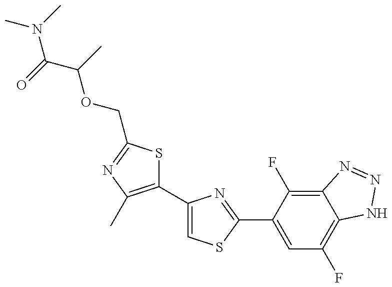 [SMILES: Cc1nc(COC(C)C(=O)N(C)C)sc1-c1csc(-c2cc(F)c3[nH]nnc3c2F)n1] |
| 51 | 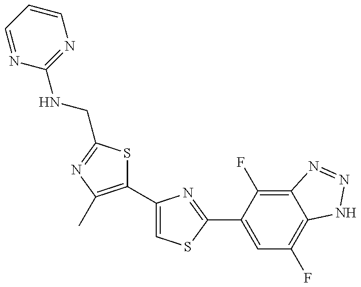 [SMILES: Cc1nc(CNc2ncccn2)sc1-c1csc(-c2cc(F)c3[nH]nnc3c2F)n1] |
| 52 | 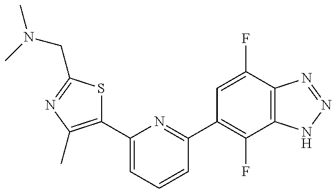 [SMILES: Cc1nc(CN(C)C)sc1-c1cccc(-c2cc(F)c3nn[nH]c3c2F)n1] |
| 53 | 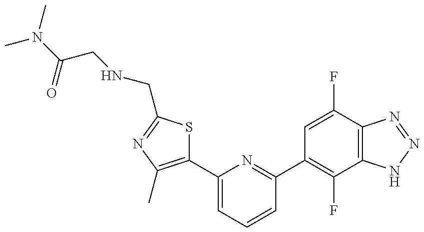 [SMILES: Cc1nc(CNCC(=O)N(C)C)sc1-c1cccc(-c2cc(F)c3nn[nH]c3c2F)n1] |
| 54 | 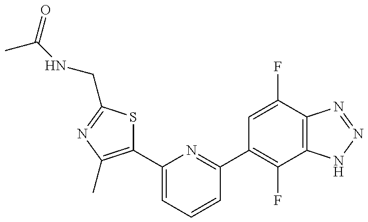 [SMILES: CC(=O)NCc1nc(C)c(-c2cccc(-c3cc(F)c4nn[nH]c4c3F)n2)s1] |
| 55 | 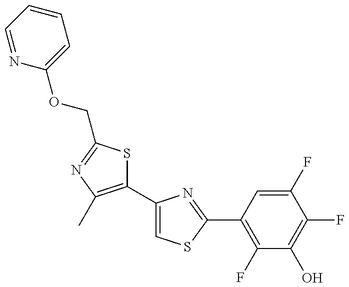 [SMILES: Cc1nc(COc2ccccn2)sc1-c1csc(-c2cc(F)c(F)c(O)c2F)n1] |
| 56 | 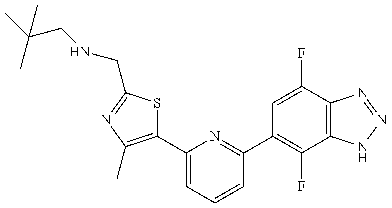 [SMILES: Cc1nc(CNCC(C)(C)C)sc1-c1cccc(-c2cc(F)c3nn[nH]c3c2F)n1] |
| 57 | 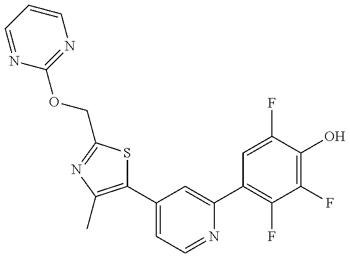 [SMILES: Cc1nc(COc2ncccn2)sc1-c1ccnc(-c2cc(F)c(O)c(F)c2F)c1] |
| 58 | 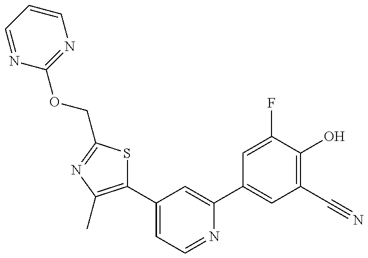 [SMILES: Cc1nc(COc2ncccn2)sc1-c1ccnc(-c2cc(F)c(O)c(C#N)c2)c1] |
| 59 | 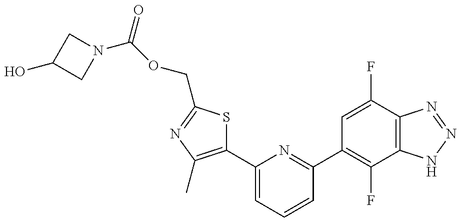 [SMILES: Cc1nc(COC(=O)N2CC(O)C2)sc1-c1cccc(-c2cc(F)c3nn[nH]c3c2F)n1] |
| 60 | 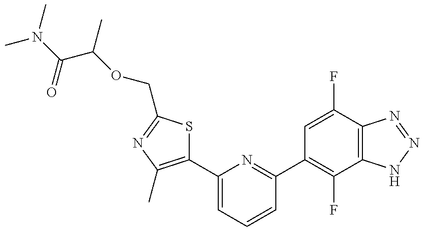 [SMILES: Cc1nc(COC(C)C(=O)N(C)C)sc1-c1cccc(-c2cc(F)c3nn[nH]c3c2F)n1] |
| 61 | 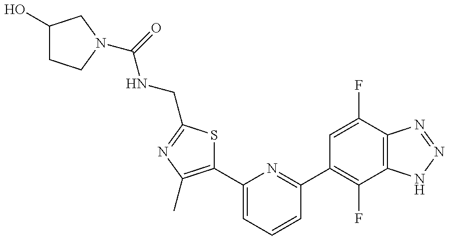 [SMILES: Cc1nc(CNC(=O)N2CCC(O)C2)sc1-c1cccc(-c2cc(F)c3nn[nH]c3c2F)n1] |
| 62 | 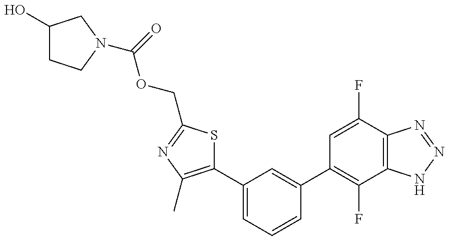 [SMILES: Cc1nc(COC(=O)N2CCC(O)C2)sc1-c1cccc(-c2cc(F)c3nn[nH]c3c2F)c1] |
| 63 | 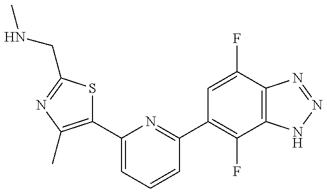 [SMILES: CNCc1nc(C)c(-c2cccc(-c3cc(F)c4nn[nH]c4c3F)n2)s1] |
| 64 | 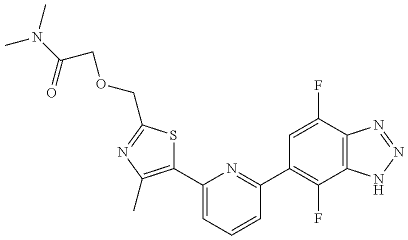 [SMILES: Cc1nc(COCC(=O)N(C)C)sc1-c1cccc(-c2cc(F)c3nn[nH]c3c2F)n1] |
| 65 | 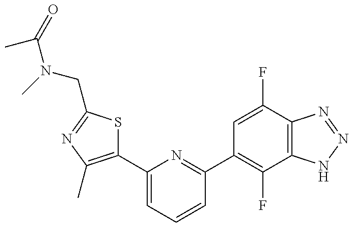 [SMILES: CC(=O)N(C)Cc1nc(C)c(-c2cccc(-c3cc(F)c4nn[nH]c4c3F)n2)s1] |
| 66 | 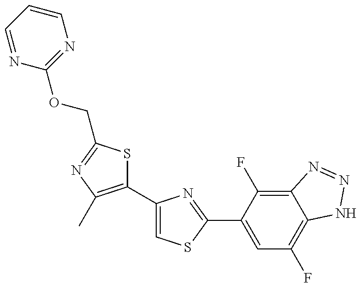 [SMILES: Cc1nc(COc2ncccn2)sc1-c1csc(-c2cc(F)c3[nH]nnc3c2F)n1] |
| 67 | 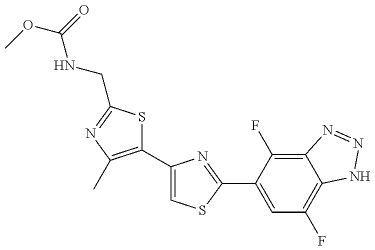 [SMILES: COC(=O)NCc1nc(C)c(-c2csc(-c3cc(F)c4[nH]nnc4c3F)n2)s1] |
| 68 | 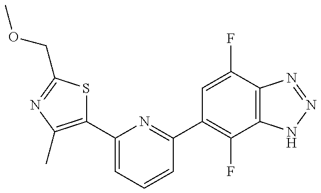 [SMILES: COCc1nc(C)c(-c2cccc(-c3cc(F)c4nn[nH]c4c3F)n2)s1] |
| 69 | 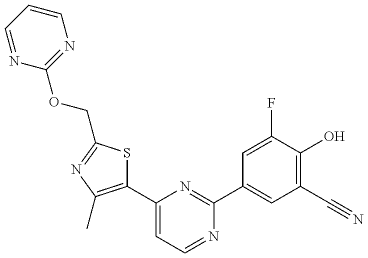 [SMILES: Cc1nc(COc2ncccn2)sc1-c1ccnc(-c2cc(F)c(O)c(C#N)c2)n1] |
| 70 | 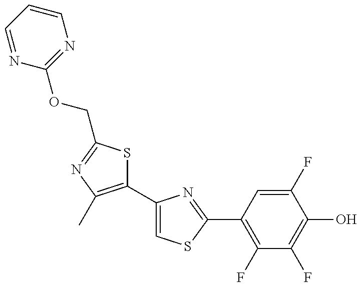 [SMILES: Cc1nc(COc2ncccn2)sc1-c1csc(-c2cc(F)c(O)c(F)c2F)n1] |
| 71 | 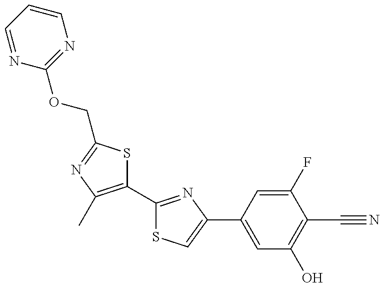 [SMILES: Cc1nc(COc2ncccn2)sc1-c1nc(-c2cc(O)c(C#N)c(F)c2)cs1] / |
| 72 | 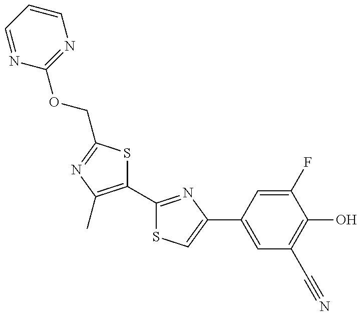 [SMILES: Cc1nc(COc2ncccn2)sc1-c1nc(-c2cc(F)c(O)c(C#N)c2)cs1] |
| 73 | 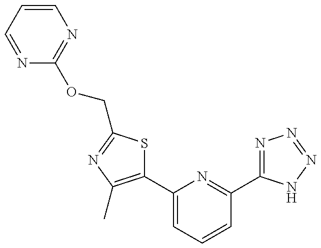 [SMILES: Cc1nc(COc2ncccn2)sc1-c1cccc(-c2nnn[nH]2)n1] |
| 74 | 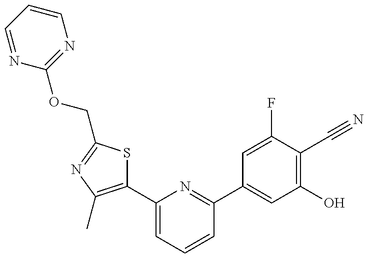 [SMILES: Cc1nc(COc2ncccn2)sc1-c1cccc(-c2cc(O)c(C#N)c(F)c2)n1] |
| 75 | 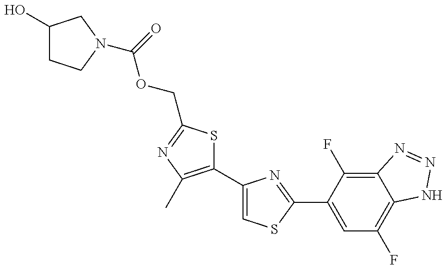 [SMILES: Cc1nc(COC(=O)N2CCC(O)C2)sc1-c1csc(-c2cc(F)c3[nH]nnc3c2F)n1] |
| 76 | 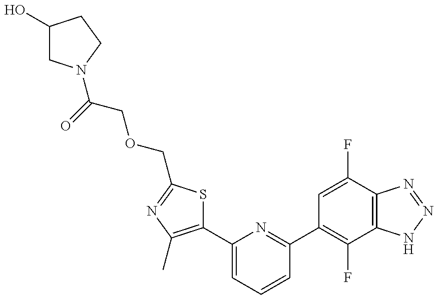 [SMILES: Cc1nc(COCC(=O)N2CCC(O)C2)sc1-c1cccc(-c2cc(F)c3nn[nH]c3c2F)n1] |
| 77 | 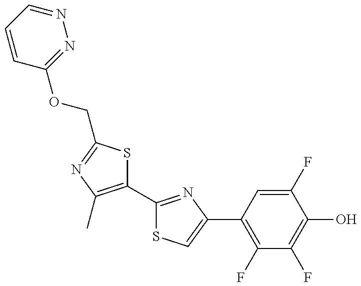 [SMILES: Cc1nc(COc2cccnn2)sc1-c1nc(-c2cc(F)c(O)c(F)c2F)cs1] |
| 78 | 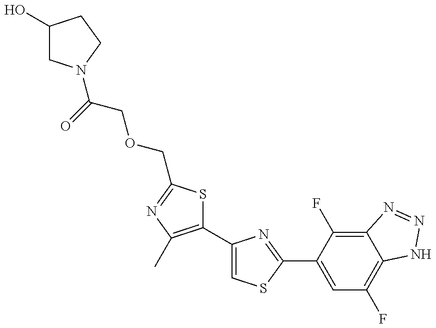 [SMILES: Cc1nc(COCC(=O)N2CCC(O)C2)sc1-c1csc(-c2cc(F)c3[nH]nnc3c2F)n1] |
| 79 | 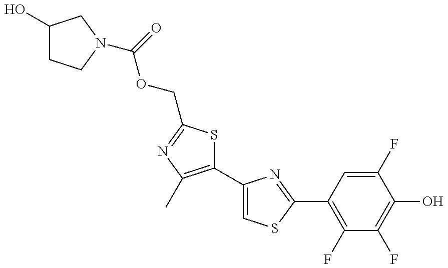 [SMILES: Cc1nc(COC(=O)N2CCC(O)C2)sc1-c1csc(-c2cc(F)c(O)c(F)c2F)n1] |
| 80 | 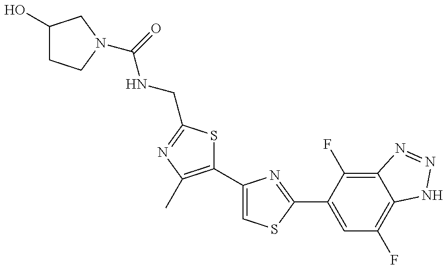 [SMILES: Cc1nc(CNC(=O)N2CCC(O)C2)sc1-c1csc(-c2cc(F)c3[nH]nnc3c2F)n1] |
| 81 | 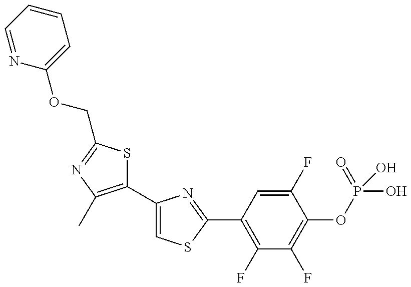 [SMILES: Cc1nc(COc2ccccn2)sc1-c1csc(-c2cc(F)c(OP(=O)(O)O)c(F)c2F)n1] |
| 82 | 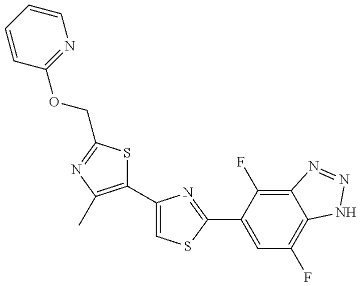 [SMILES: Cc1nc(COc2ccccn2)sc1-c1csc(-c2cc(F)c3[nH]nnc3c2F)n1] |
| 83 | 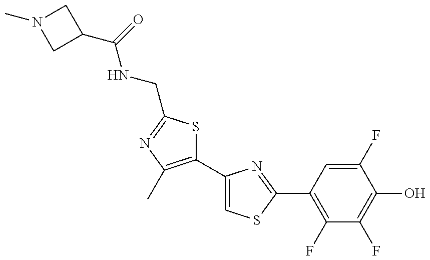 [SMILES: Cc1nc(CNC(=O)C2CN(C)C2)sc1-c1csc(-c2cc(F)c(O)c(F)c2F)n1] |
| 84 | 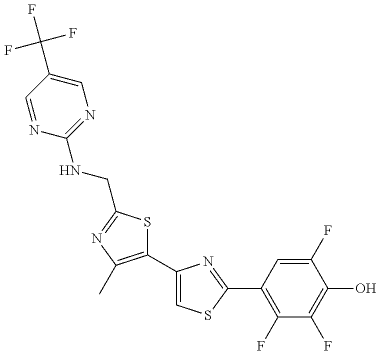 [SMILES: Cc1nc(CNc2ncc(C(F)(F)F)cn2)sc1-c1csc(-c2cc(F)c(O)c(F)c2F)n1] |
| 85 | 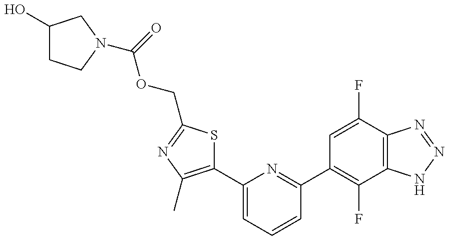 [SMILES: Cc1nc(COC(=O)N2CCC(O)C2)sc1-c1cccc(-c2cc(F)c3nn[nH]c3c2F)n1] |
| 86 | 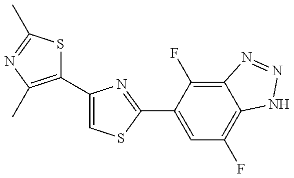 [SMILES: Cc1nc(C)c(-c2csc(-c3cc(F)c4[nH]nnc4c3F)n2)s1] |
| 87 | 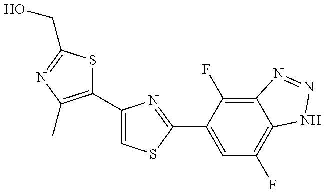 [SMILES: Cc1nc(CO)sc1-c1csc(-c2cc(F)c3[nH]nnc3c2F)n1] |
| 88 | 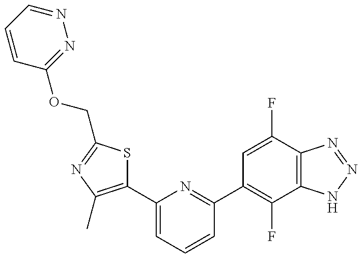 [SMILES: Cc1nc(COc2cccnn2)sc1-c1cccc(-c2cc(F)c3nn[nH]c3c2F)n1] |
| 89 | 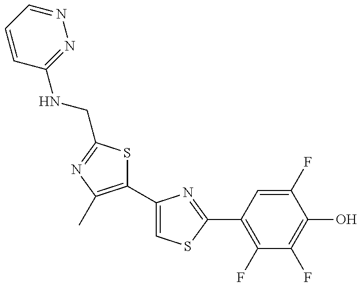 [SMILES: Cc1nc(CNc2cccnn2)sc1-c1csc(-c2cc(F)c(O)c(F)c2F)n1] |
| 90 | 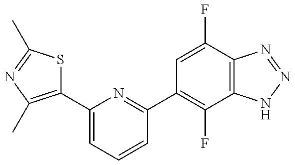 [SMILES: Cc1nc(C)c(-c2cccc(-c3cc(F)c4nn[nH]c4c3F)n2)s1] |
| 91 | 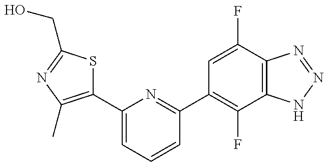 [SMILES: Cc1nc(CO)sc1-c1cccc(-c2cc(F)c3nn[nH]c3c2F)n1] |
| 92 | 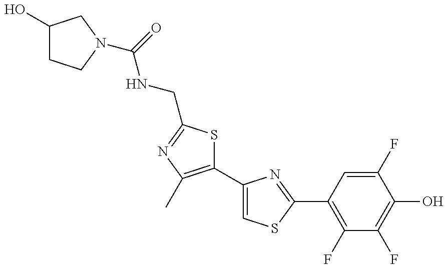 [SMILES: Cc1nc(CNC(=O)N2CCC(O)C2)sc1-c1csc(-c2cc(F)c(O)c(F)c2F)n1] |
| 93 | 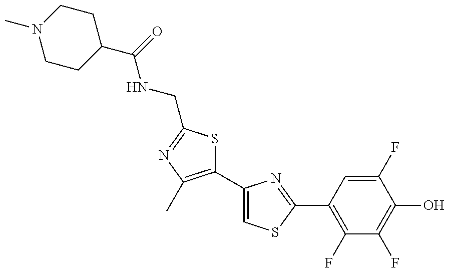 [SMILES: Cc1nc(CNC(=O)C2CCN(C)CC2)sc1-c1csc(-c2cc(F)c(O)c(F)c2F)n1] |
| 94 | 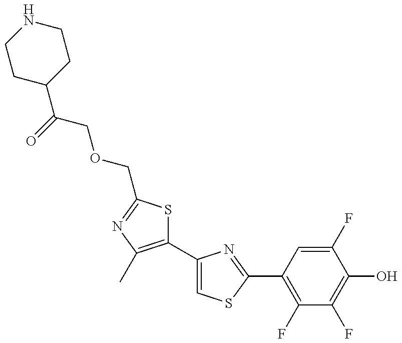 [SMILES: Cc1nc(COCC(=O)C2CCNCC2)sc1-c1csc(-c2cc(F)c(O)c(F)c2F)n1] |
| 95 | 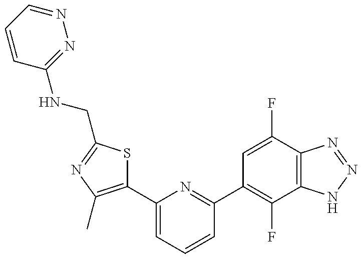 [SMILES: Cc1nc(CNc2cccnn2)sc1-c1cccc(-c2cc(F)c3nn[nH]c3c2F)n1] |
| 96 | 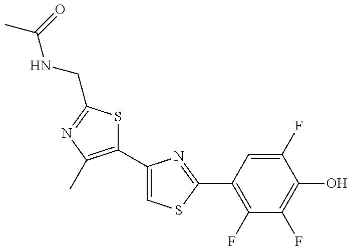 [SMILES: CC(=O)NCc1nc(C)c(-c2csc(-c3cc(F)c(O)c(F)c3F)n2)s1] |
| 97 | 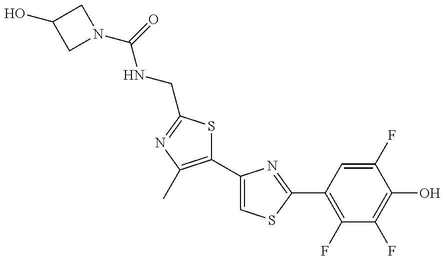 [SMILES: Cc1nc(CNC(=O)N2CC(O)C2)sc1-c1csc(-c2cc(F)c(O)c(F)c2F)n1] |
| 98 | 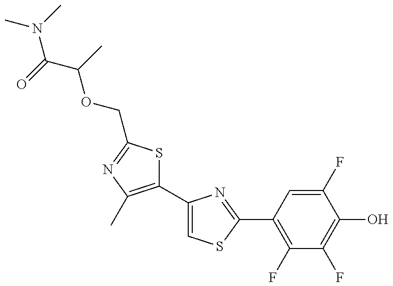 [SMILES: Cc1nc(COC(C)C(=O)N(C)C)sc1-c1csc(-c2cc(F)c(O)c(F)c2F)n1] |
| 99 | 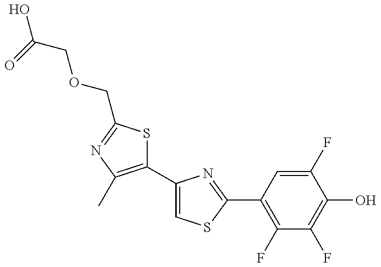 [SMILES: Cc1nc(COCC(=O)O)sc1-c1csc(-c2cc(F)c(O)c(F)c2F)n1] |
| 100 | 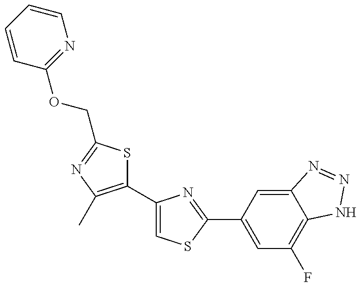 [SMILES: Cc1nc(COc2ccccn2)sc1-c1csc(-c2cc(F)c3[nH]nnc3c2)n1] |
| 101 | 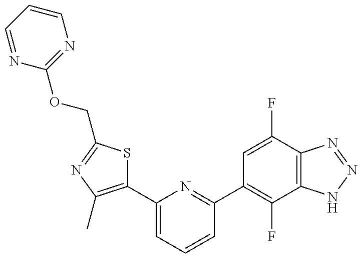 [SMILES: Cc1nc(COc2ncccn2)sc1-c1cccc(-c2cc(F)c3nn[nH]c3c2F)n1] |
| 102 | 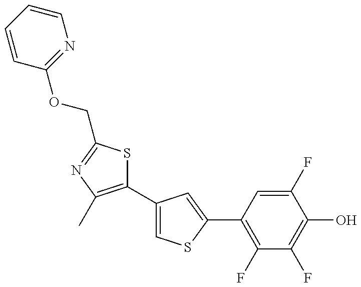 [SMILES: Cc1nc(COc2ccccn2)sc1-c1csc(-c2cc(F)c(O)c(F)c2F)c1] |
| 103 | 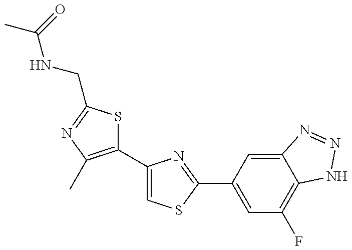 [SMILES: CC(=O)NCc1nc(C)c(-c2csc(-c3cc(F)c4[nH]nnc4c3)n2)s1] |
| 104 | 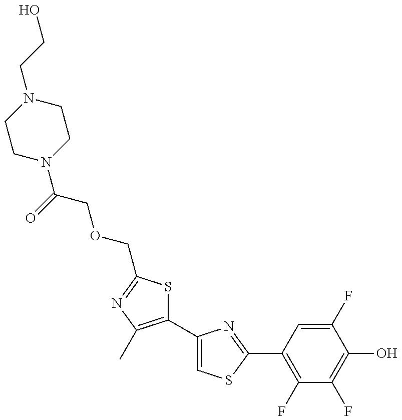 [SMILES: Cc1nc(COCC(=O)N2CCN(CCO)CC2)sc1-c1csc(-c2cc(F)c(O)c(F)c2F)n1] |
| 105 | 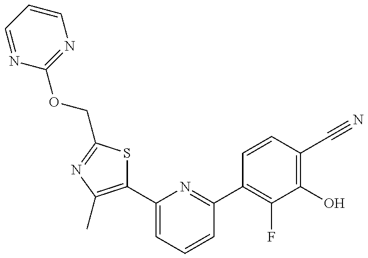 [SMILES: Cc1nc(COc2ncccn2)sc1-c1cccc(-c2ccc(C#N)c(O)c2F)n1] |
| 106 | 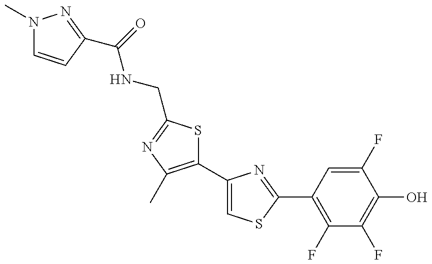 [SMILES: Cc1nc(CNC(=O)c2ccn(C)n2)sc1-c1csc(-c2cc(F)c(O)c(F)c2F)n1] |
| 107 | 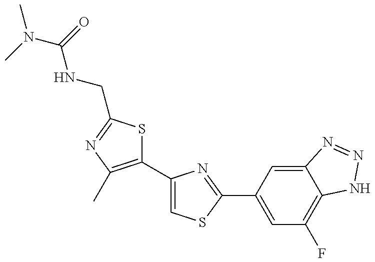 [SMILES: Cc1nc(CNC(=O)N(C)C)sc1-c1csc(-c2cc(F)c3[nH]nnc3c2)n1] |
| 108 | 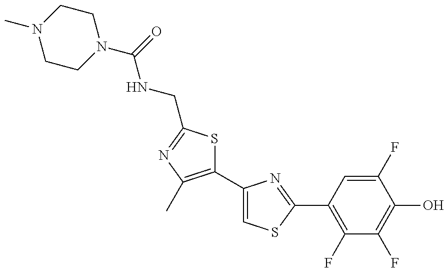 [SMILES: Cc1nc(CNC(=O)N2CCN(C)CC2)sc1-c1csc(-c2cc(F)c(O)c(F)c2F)n1] |
| 109 | 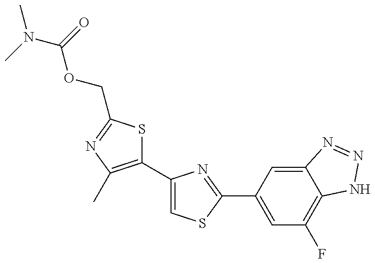 [SMILES: Cc1nc(COC(=O)N(C)C)sc1-c1csc(-c2cc(F)c3[nH]nnc3c2)n1] |
| 110 | 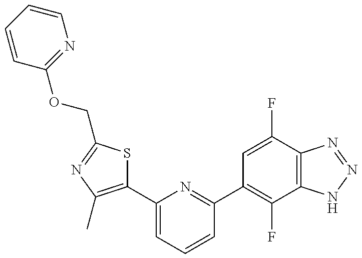 [SMILES: Cc1nc(COc2ccccn2)sc1-c1cccc(-c2cc(F)c3nn[nH]c3c2F)n1] |
| 111 | 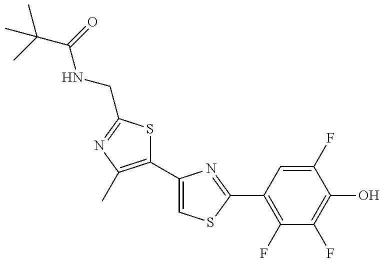 [SMILES: Cc1nc(CNC(=O)C(C)(C)C)sc1-c1csc(-c2cc(F)c(O)c(F)c2F)n1] |
| 112 | 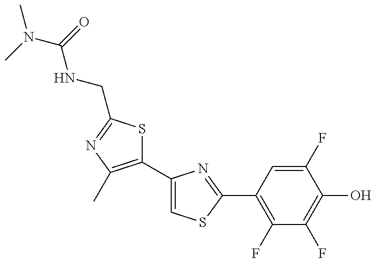 [SMILES: Cc1nc(CNC(=O)N(C)C)sc1-c1csc(-c2cc(F)c(O)c(F)c2F)n1] |
| 113 | 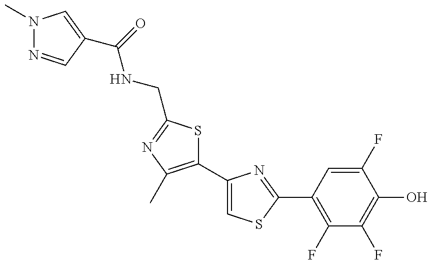 [SMILES: Cc1nc(CNC(=O)c2cnn(C)c2)sc1-c1csc(-c2cc(F)c(O)c(F)c2F)n1] |
| 114 | 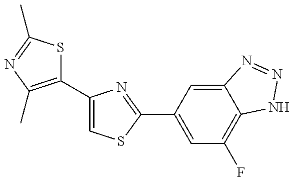 [SMILES: Cc1nc(C)c(-c2csc(-c3cc(F)c4[nH]nnc4c3)n2)s1] |
| 115 | 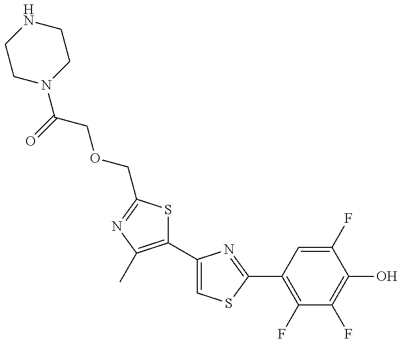 [SMILES: Cc1nc(COCC(=O)N2CCNCC2)sc1-c1csc(-c2cc(F)c(O)c(F)c2F)n1] |
| 116 | 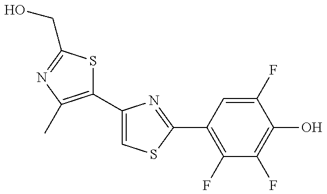 [SMILES: Cc1nc(CO)sc1-c1csc(-c2cc(F)c(O)c(F)c2F)n1] |
| 117 | 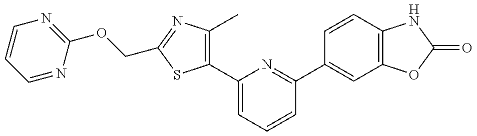 [SMILES: Cc1nc(COc2ncccn2)sc1-c1cccc(-c2ccc3[nH]c(=O)oc3c2)n1] |
| 118 | 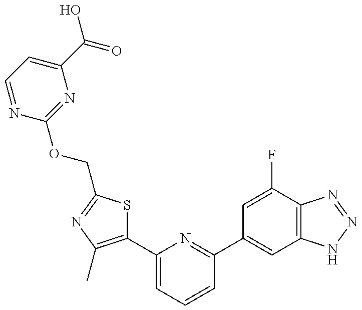 [SMILES: Cc1nc(COc2nccc(C(=O)O)n2)sc1-c1cccc(-c2cc(F)c3nn[nH]c3c2)n1] |
| 119 | 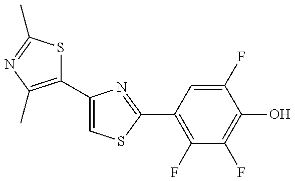 [SMILES: Cc1nc(C)c(-c2csc(-c3cc(F)c(O)c(F)c3F)n2)s1] |
| 120 | 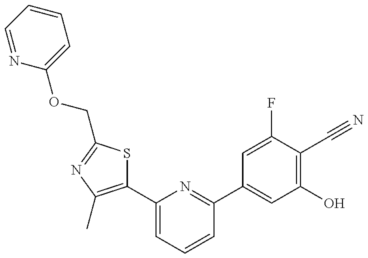 [SMILES: Cc1nc(COc2ccccn2)sc1-c1cccc(-c2cc(O)c(C#N)c(F)c2)n1] |
| 121 | 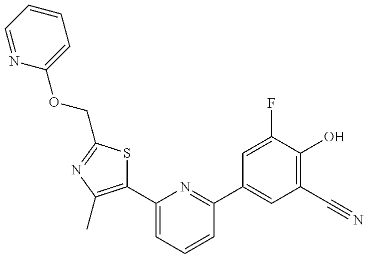 [SMILES: Cc1nc(COc2ccccn2)sc1-c1cccc(-c2cc(F)c(O)c(C#N)c2)n1] |
| 122 | 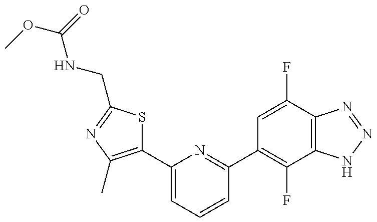 [SMILES: COC(=O)NCc1nc(C)c(-c2cccc(-c3cc(F)c4nn[nH]c4c3F)n2)s1] |
| 123 | 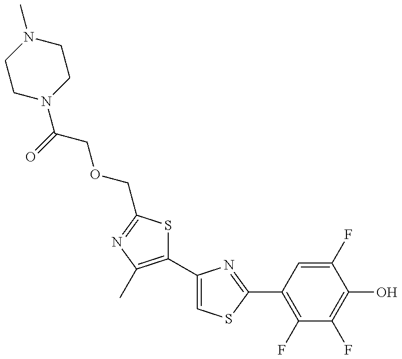 [SMILES: Cc1nc(COCC(=O)N2CCN(C)CC2)sc1-c1csc(-c2cc(F)c(O)c(F)c2F)n1] |
| 124 | 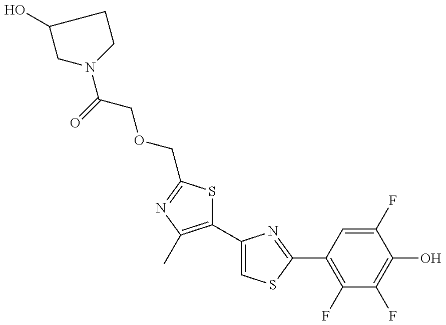 [SMILES: Cc1nc(COCC(=O)N2CCC(O)C2)sc1-c1csc(-c2cc(F)c(O)c(F)c2F)n1] |
| 125 | 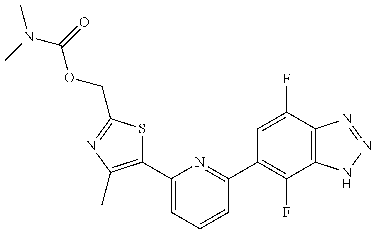 [SMILES: Cc1nc(COC(=O)N(C)C)sc1-c1cccc(-c2cc(F)c3nn[nH]c3c2F)n1] |
| 126 | 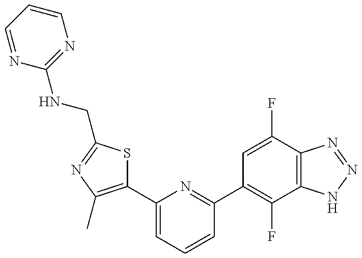 [SMILES: Cc1nc(CNc2ncccn2)sc1-c1cccc(-c2cc(F)c3nn[nH]c3c2F)n1] |
| 127 | 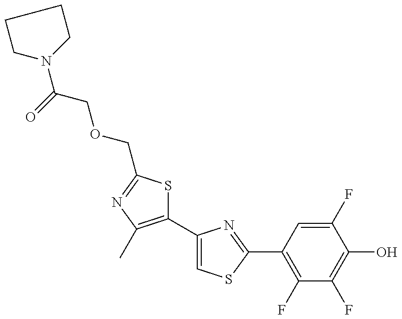 [SMILES: Cc1nc(COCC(=O)N2CCCC2)sc1-c1csc(-c2cc(F)c(O)c(F)c2F)n1] |
| 128 | 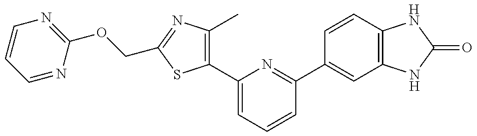 [SMILES: Cc1nc(COc2ncccn2)sc1-c1cccc(-c2ccc3[nH]c(=O)[nH]c3c2)n1] |
| 129 | 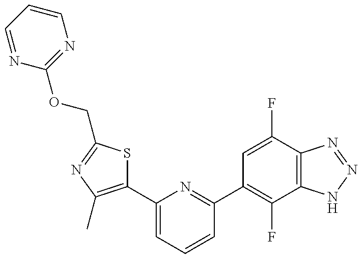 [SMILES: Cc1nc(COc2ncccn2)sc1-c1cccc(-c2cc(F)c3nn[nH]c3c2F)n1] |
| 130 | 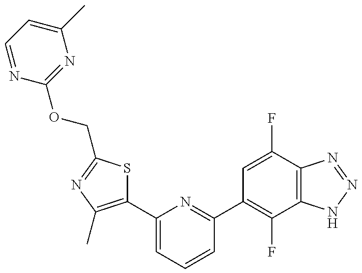 [SMILES: Cc1ccnc(OCc2nc(C)c(-c3cccc(-c4cc(F)c5nn[nH]c5c4F)n3)s2)n1] |
| 131 | 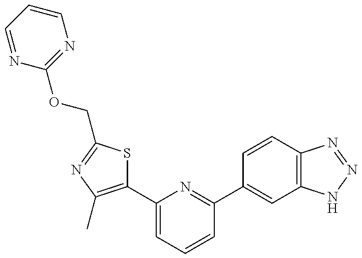 [SMILES: Cc1nc(COc2ncccn2)sc1-c1cccc(-c2ccc3nn[nH]c3c2)n1] |
| 132 | 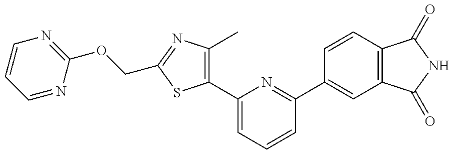 [SMILES: Cc1nc(COc2ncccn2)sc1-c1cccc(-c2ccc3c(c2)C(=O)NC3=O)n1] |
| 133 | 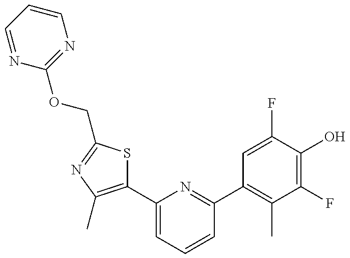 [SMILES: Cc1nc(COc2ncccn2)sc1-c1cccc(-c2cc(F)c(O)c(F)c2C)n1] |
| 134 | 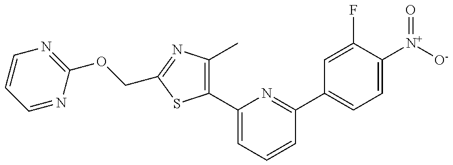 [SMILES: Cc1nc(COc2ncccn2)sc1-c1cccc(-c2ccc([N+](=O)[O-])c(F)c2)n1] |
| 135 | 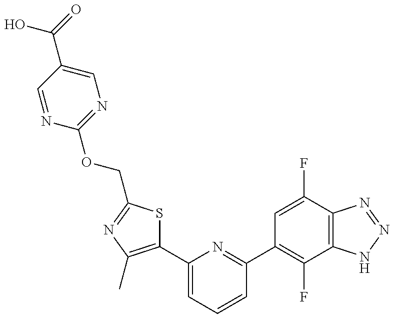 [SMILES: Cc1nc(COc2ncc(C(=O)O)cn2)sc1-c1cccc(-c2cc(F)c3nn[nH]c3c2F)n1] |
| 136 | 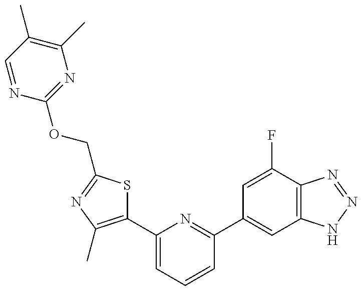 [SMILES: Cc1cnc(OCc2nc(C)c(-c3cccc(-c4cc(F)c5nn[nH]c5c4)n3)s2)nc1C] |
| 137 | 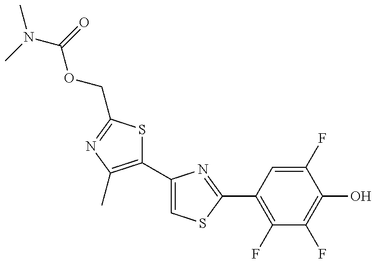 [SMILES: Cc1nc(COC(=O)N(C)C)sc1-c1csc(-c2cc(F)c(O)c(F)c2F)n1] |
| 138 | 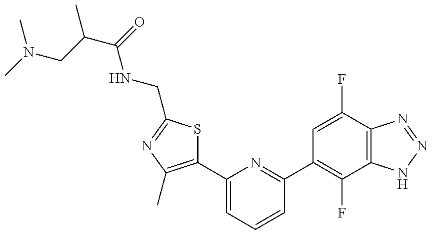 [SMILES: Cc1nc(CNC(=O)C(C)CN(C)C)sc1-c1cccc(-c2cc(F)c3nn[nH]c3c2F)n1] |
| 139 | 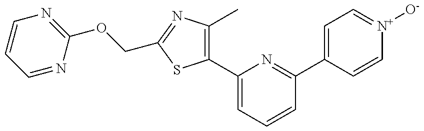 [SMILES: Cc1nc(COc2ncccn2)sc1-c1cccc(-c2cc[n+]([O-])cc2)n1] |
| 140 | 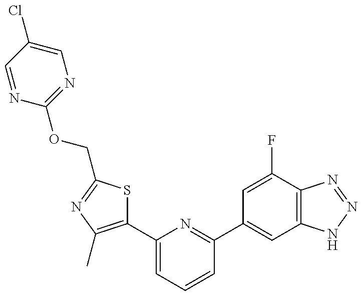 [SMILES: Cc1nc(COc2ncc(Cl)cn2)sc1-c1cccc(-c2cc(F)c3nn[nH]c3c2)n1] |
| 141 | 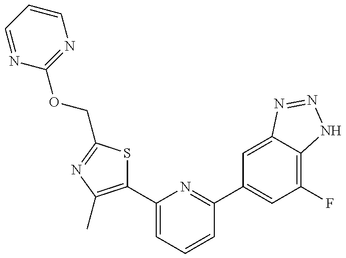 [SMILES: Cc1nc(COc2ncccn2)sc1-c1cccc(-c2cc(F)c3[nH]nnc3c2)n1] |
| 142 | 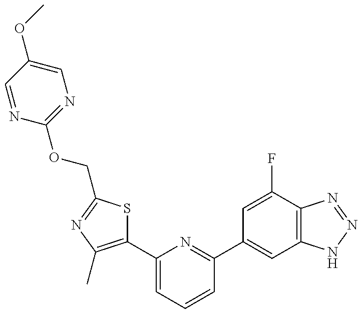 [SMILES: COc1cnc(OCc2nc(C)c(-c3cccc(-c4cc(F)c5nn[nH]c5c4)n3)s2)nc1] |
| 143 | 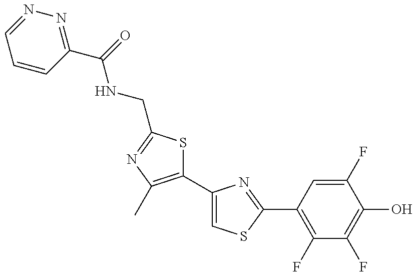 [SMILES: Cc1nc(CNC(=O)c2cccnn2)sc1-c1csc(-c2cc(F)c(O)c(F)c2F)n1] |
| 144 | 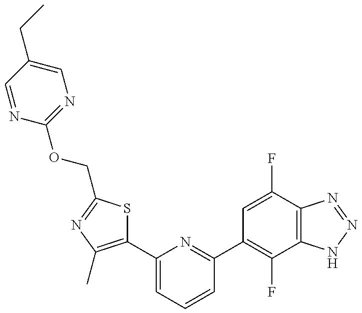 [SMILES: CCc1cnc(OCc2nc(C)c(-c3cccc(-c4cc(F)c5nn[nH]c5c4F)n3)s2)nc1] |
| 145 | 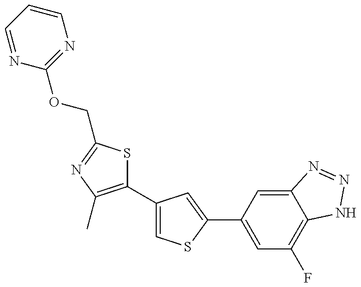 [SMILES: Cc1nc(COc2ncccn2)sc1-c1csc(-c2cc(F)c3[nH]nnc3c2)c1] |
| 146 | 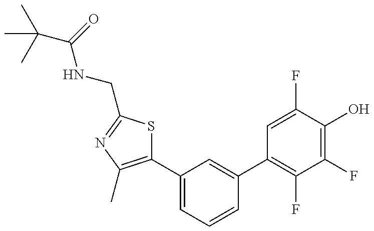 [SMILES: Cc1nc(CNC(=O)C(C)(C)C)sc1-c1cccc(-c2cc(F)c(O)c(F)c2F)c1] |
| 147 | 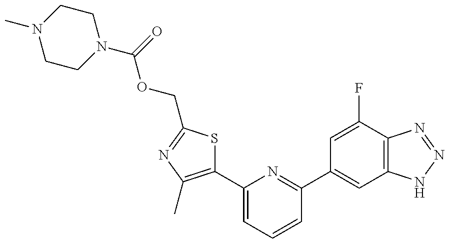 [SMILES: Cc1nc(COC(=O)N2CCN(C)CC2)sc1-c1cccc(-c2cc(F)c3nn[nH]c3c2)n1] |
| 148 | 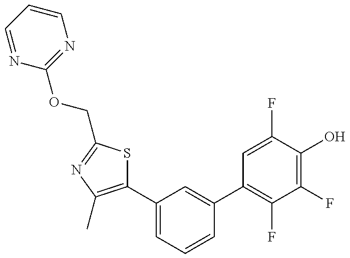 [SMILES: Cc1nc(COc2ncccn2)sc1-c1cccc(-c2cc(F)c(O)c(F)c2F)c1] |
| 149 | 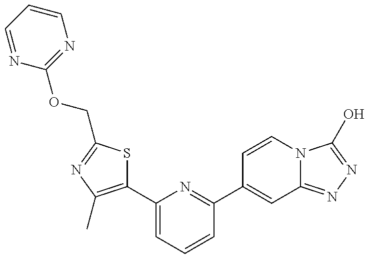 [SMILES: Cc1nc(COc2ncccn2)sc1-c1cccc(-c2ccn3c(O)nnc3c2)n1] |
| 150 | 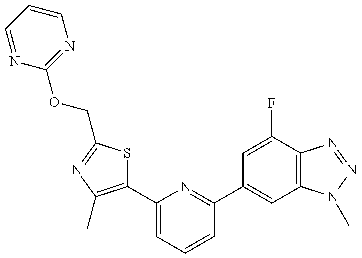 [SMILES: Cc1nc(COc2ncccn2)sc1-c1cccc(-c2cc(F)c3nnn(C)c3c2)n1] |
| 151 | 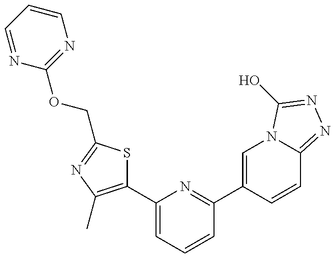 [SMILES: Cc1nc(COc2ncccn2)sc1-c1cccc(-c2ccc3nnc(O)n3c2)n1] |
| 152 | 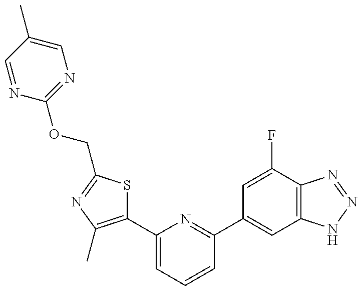 [SMILES: Cc1cnc(OCc2nc(C)c(-c3cccc(-c4cc(F)c5nn[nH]c5c4)n3)s2)nc1] |
| 153 | 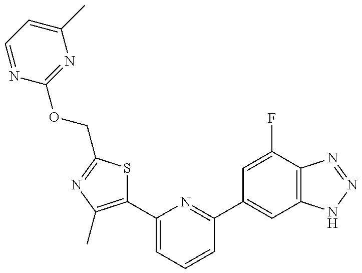 [SMILES: Cc1ccnc(OCc2nc(C)c(-c3cccc(-c4cc(F)c5nn[nH]c5c4)n3)s2)n1] |
| 154 | 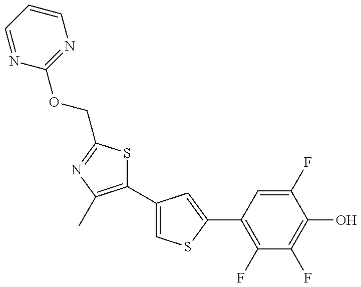 [SMILES: Cc1nc(COc2ncccn2)sc1-c1csc(-c2cc(F)c(O)c(F)c2F)c1] |
| 155 | 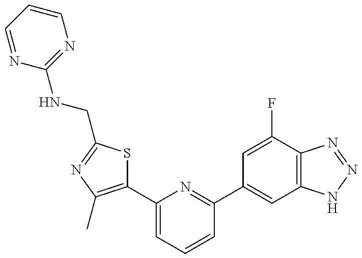 [SMILES: Cc1nc(CNc2ncccn2)sc1-c1cccc(-c2cc(F)c3nn[nH]c3c2)n1] |
| 156 | 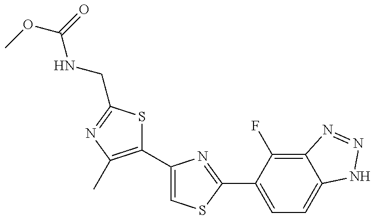 [SMILES: COC(=O)NCc1nc(C)c(-c2csc(-c3ccc4[nH]nnc4c3F)n2)s1] |
| 157 | 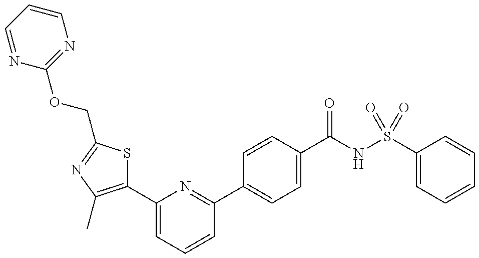 [SMILES: Cc1nc(COc2ncccn2)sc1-c1cccc(-c2ccc(C(=O)NS(=O)(=O)c3ccccc3)cc2)n1] |
| 158 | 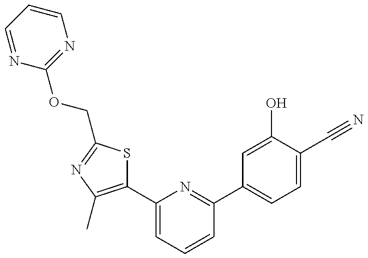 [SMILES: Cc1nc(COc2ncccn2)sc1-c1cccc(-c2ccc(C#N)c(O)c2)n1] |
| 159 | 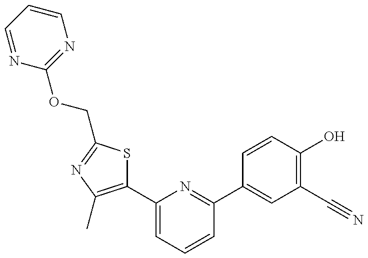 [SMILES: Cc1nc(COc2ncccn2)sc1-c1cccc(-c2ccc(O)c(C#N)c2)n1] |
| 160 | 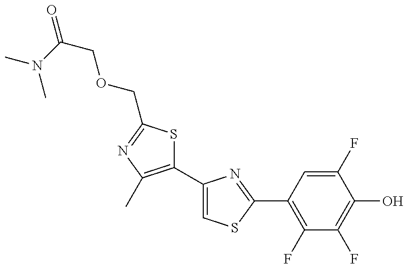 [SMILES: Cc1nc(COCC(=O)N(C)C)sc1-c1csc(-c2cc(F)c(O)c(F)c2F)n1] |
| 161 | 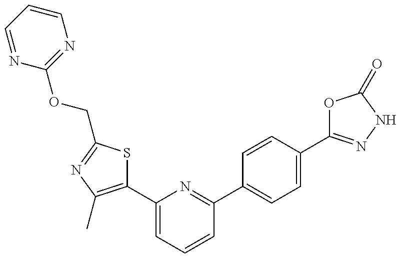 [SMILES: Cc1nc(COc2ncccn2)sc1-c1cccc(-c2ccc(-c3n[nH]c(=O)o3)cc2)n1] |
| 162 | 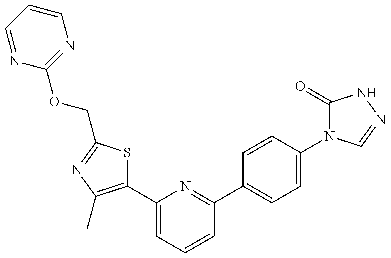 [SMILES: Cc1nc(COc2ncccn2)sc1-c1cccc(-c2ccc(-n3cn[nH]c3=O)cc2)n1] |
| 163 | 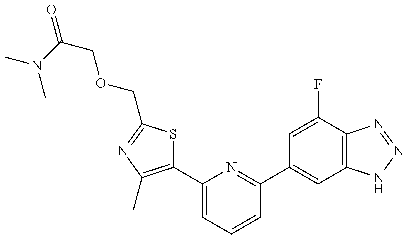 [SMILES: Cc1nc(COCC(=O)N(C)C)sc1-c1cccc(-c2cc(F)c3nn[nH]c3c2)n1] |
| 164 | 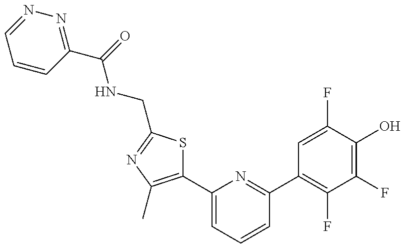 [SMILES: Cc1nc(CNC(=O)c2cccnn2)sc1-c1cccc(-c2cc(F)c(O)c(F)c2F)n1] |
| 165 | 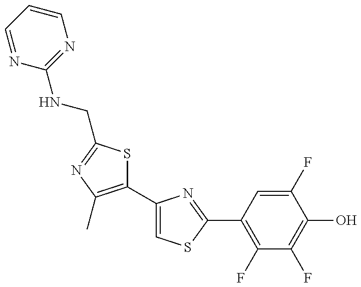 [SMILES: Cc1nc(CNc2ncccn2)sc1-c1csc(-c2cc(F)c(O)c(F)c2F)n1] |
| 166 | 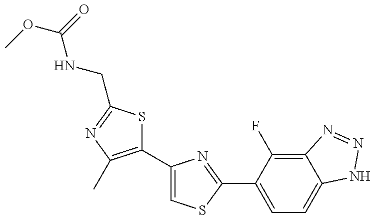 [SMILES: COC(=O)NCc1nc(C)c(-c2csc(-c3ccc4[nH]nnc4c3F)n2)s1] |
| 167 | 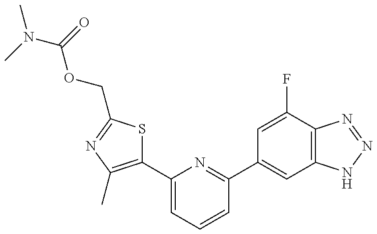 [SMILES: Cc1nc(COC(=O)N(C)C)sc1-c1cccc(-c2cc(F)c3nn[nH]c3c2)n1] |
| 168 | 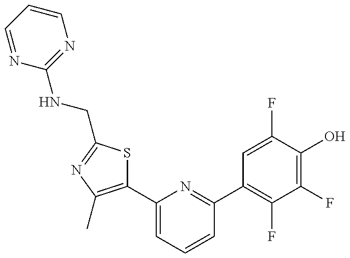 [SMILES: Cc1nc(CNc2ncccn2)sc1-c1cccc(-c2cc(F)c(O)c(F)c2F)n1] |
| 169 | 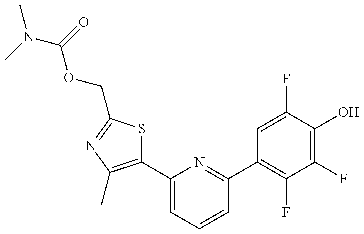 [SMILES: Cc1nc(COC(=O)N(C)C)sc1-c1cccc(-c2cc(F)c(O)c(F)c2F)n1] |
| 170 | 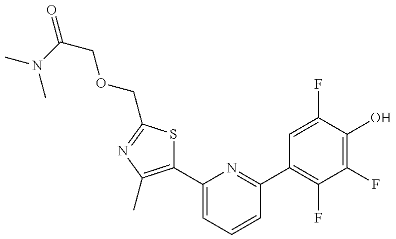 [SMILES: Cc1nc(COCC(=O)N(C)C)sc1-c1cccc(-c2cc(F)c(O)c(F)c2F)n1] / |
| 171 | 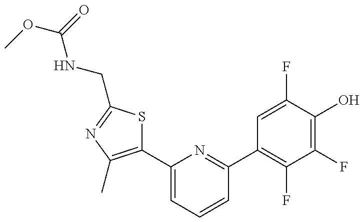 [SMILES: COC(=O)NCc1nc(C)c(-c2cccc(-c3cc(F)c(O)c(F)c3F)n2)s1] |
| 172 | 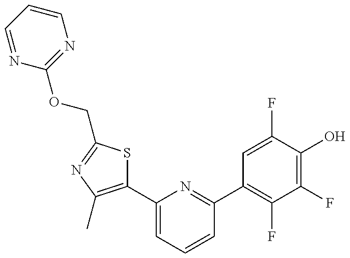 [SMILES: Cc1nc(COc2ncccn2)sc1-c1cccc(-c2cc(F)c(O)c(F)c2F)n1] |
| 173 | 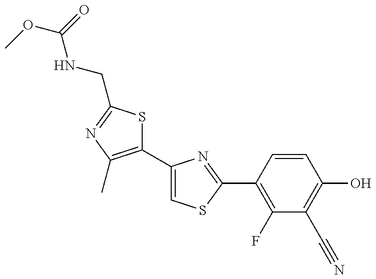 [SMILES: COC(=O)NCc1nc(C)c(-c2csc(-c3ccc(O)c(C#N)c3F)n2)s1] |
| 174 | 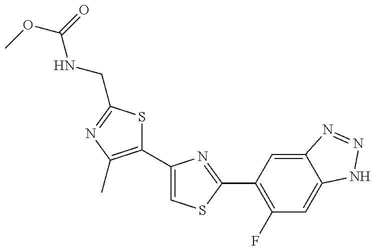 [SMILES: COC(=O)NCc1nc(C)c(-c2csc(-c3cc4nn[nH]c4cc3F)n2)s1] |
| 175 | 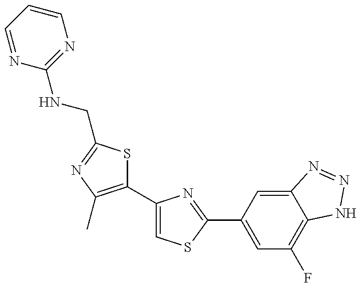 [SMILES: Cc1nc(CNc2ncccn2)sc1-c1csc(-c2cc(F)c3[nH]nnc3c2)n1] |
| 176 | 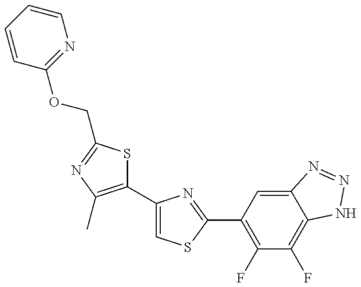 [SMILES: Cc1nc(COc2ccccn2)sc1-c1csc(-c2cc3nn[nH]c3c(F)c2F)n1] |
| 177 | 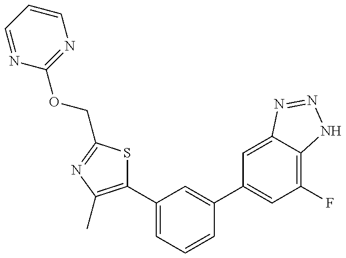 [SMILES: Cc1nc(COc2ncccn2)sc1-c1cccc(-c2cc(F)c3[nH]nnc3c2)c1] |
| 178 | 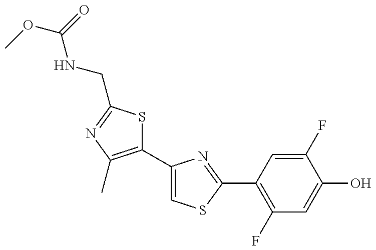 [SMILES: COC(=O)NCc1nc(C)c(-c2csc(-c3cc(F)c(O)cc3F)n2)s1] |
| 179 | 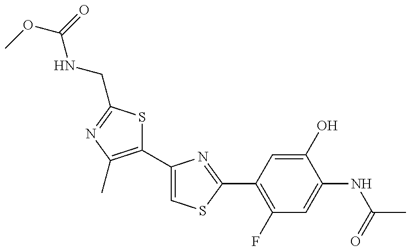 [SMILES: COC(=O)NCc1nc(C)c(-c2csc(-c3cc(O)c(NC(C)=O)cc3F)n2)s1] |
| 180 | 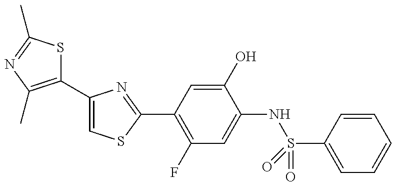 [SMILES: Cc1nc(C)c(-c2csc(-c3cc(O)c(NS(=O)(=O)c4ccccc4)cc3F)n2)s1] |
| 181 | 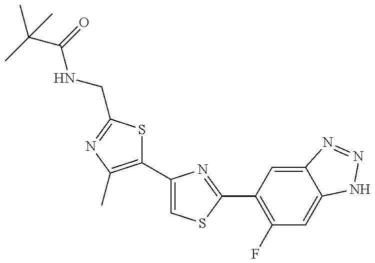 [SMILES: Cc1nc(CNC(=O)C(C)(C)C)sc1-c1csc(-c2cc3nn[nH]c3cc2F)n1] |
| 182 | 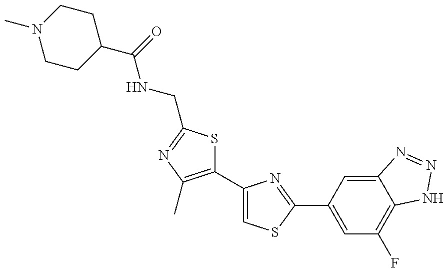 [SMILES: Cc1nc(CNC(=O)C2CCN(C)CC2)sc1-c1csc(-c2cc(F)c3[nH]nnc3c2)n1] |
| 183 | 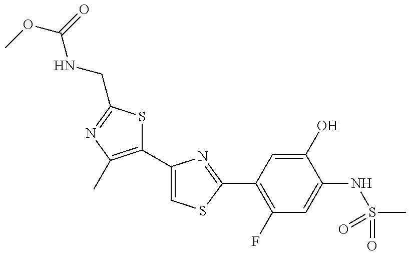 |
| 184 | 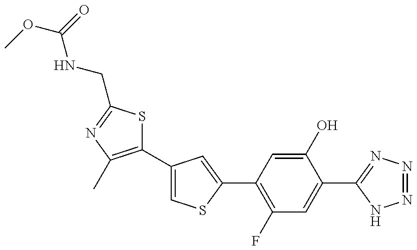 [SMILES: COC(=O)NCc1nc(C)c(-c2csc(-c3cc(O)c(-c4nnn[nH]4)cc3F)c2)s1] |
| 185 | 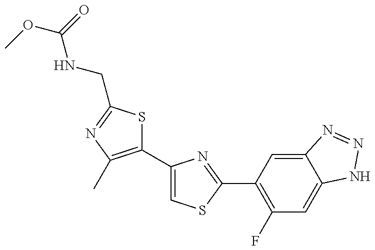 [SMILES: COC(=O)NCc1nc(C)c(-c2csc(-c3cc4nn[nH]c4cc3F)n2)s1] |
| 186 | 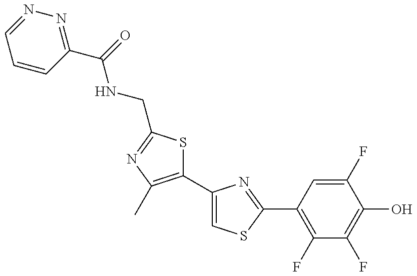 [SMILES: Cc1nc(CNC(=O)c2cccnn2)sc1-c1csc(-c2cc(F)c(O)c(F)c2F)n1] |
| 187 | 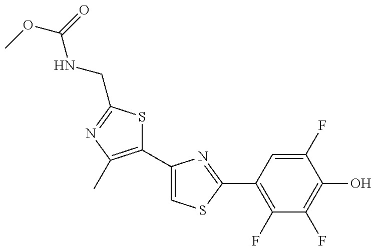 [SMILES: COC(=O)NCc1nc(C)c(-c2csc(-c3cc(F)c(O)c(F)c3F)n2)s1] |
| 188 | 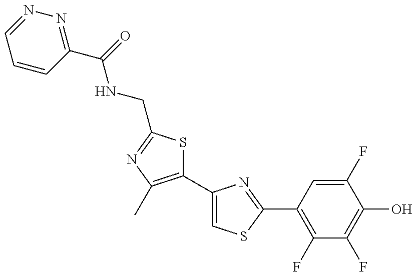 [SMILES: Cc1nc(CNC(=O)c2cccnn2)sc1-c1csc(-c2cc(F)c(O)c(F)c2F)n1] |
| 189 | 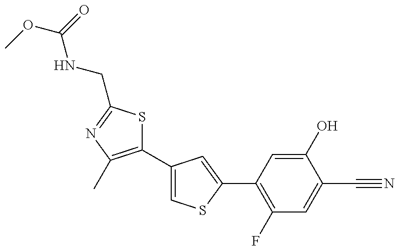 [SMILES: COC(=O)NCc1nc(C)c(-c2csc(-c3cc(O)c(C#N)cc3F)c2)s1] |
| 190 | 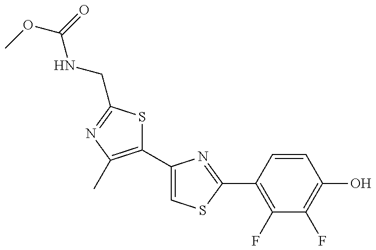 [SMILES: COC(=O)NCc1nc(C)c(-c2csc(-c3ccc(O)c(F)c3F)n2)s1] |
| 191 | 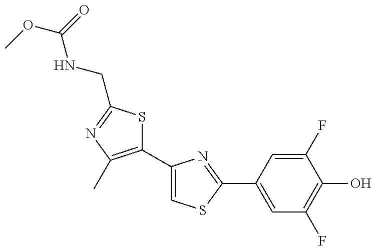 [SMILES: COC(=O)NCc1nc(C)c(-c2csc(-c3cc(F)c(O)c(F)c3)n2)s1] |
| 192 | 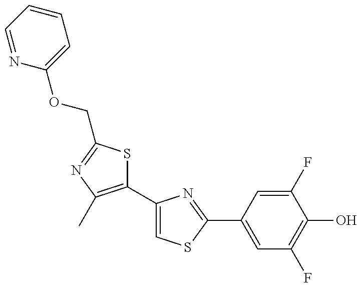 [SMILES: Cc1nc(COc2ccccn2)sc1-c1csc(-c2cc(F)c(O)c(F)c2)n1] |
| 193 | 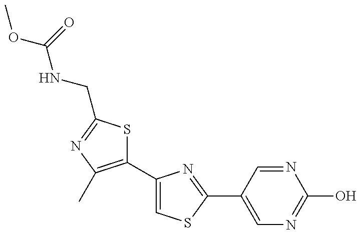 [SMILES: COC(=O)NCc1nc(C)c(-c2csc(-c3cnc(O)nc3)n2)s1] |
| 194 | 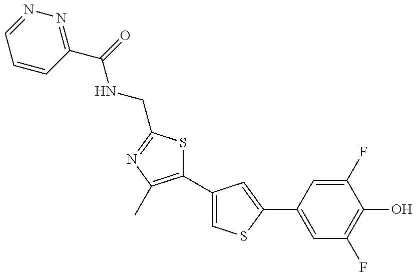 [SMILES: Cc1nc(CNC(=O)c2cccnn2)sc1-c1csc(-c2cc(F)c(O)c(F)c2)c1] |
| 195 | 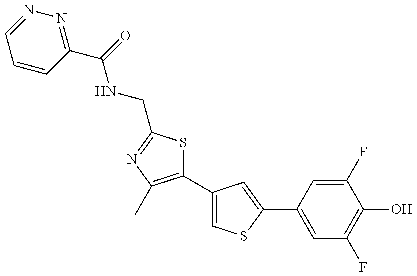 [SMILES: Cc1nc(CNC(=O)c2cccnn2)sc1-c1csc(-c2cc(F)c(O)c(F)c2)c1] |
| 196 | 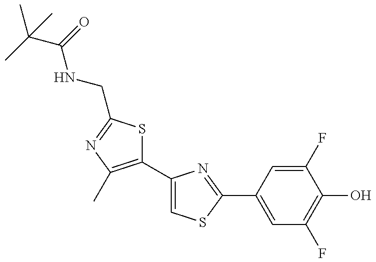 [SMILES: Cc1nc(CNC(=O)C(C)(C)C)sc1-c1csc(-c2cc(F)c(O)c(F)c2)n1] |
| 197 | 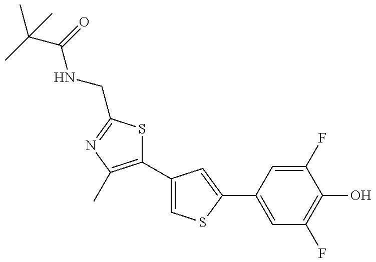 [SMILES: Cc1nc(CNC(=O)C(C)(C)C)sc1-c1csc(-c2cc(F)c(O)c(F)c2)c1] |
| 198 | 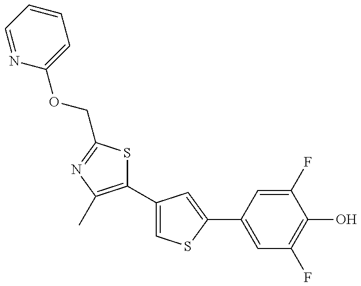 [SMILES: Cc1nc(COc2ccccn2)sc1-c1csc(-c2cc(F)c(O)c(F)c2)c1] |
| 199 | 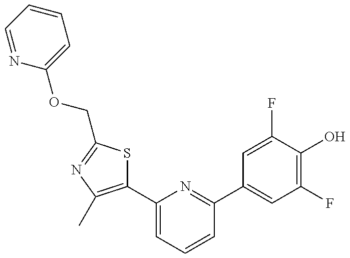 [SMILES: Cc1nc(COc2ccccn2)sc1-c1cccc(-c2cc(F)c(O)c(F)c2)n1] |
| 200 | 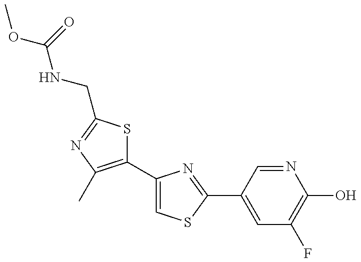 [SMILES: COC(=O)NCc1nc(C)c(-c2csc(-c3cnc(O)c(F)c3)n2)s1] |
| 201 | 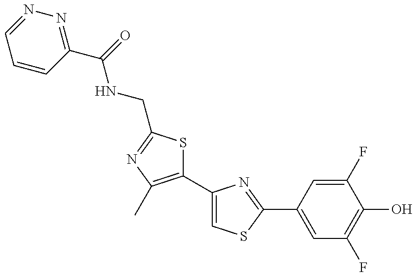 [SMILES: Cc1nc(CNC(=O)c2cccnn2)sc1-c1csc(-c2cc(F)c(O)c(F)c2)n1] |
| 202 | 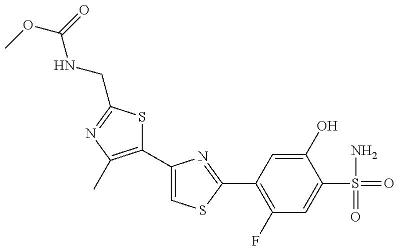 [SMILES: COC(=O)NCc1nc(C)c(-c2csc(-c3cc(O)c(S(N)(=O)=O)cc3F)n2)s1] |
| 203 | 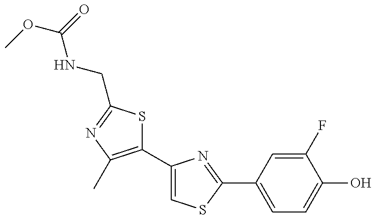 [SMILES: COC(=O)NCc1nc(C)c(-c2csc(-c3ccc(O)c(F)c3)n2)s1] |
| 204 | 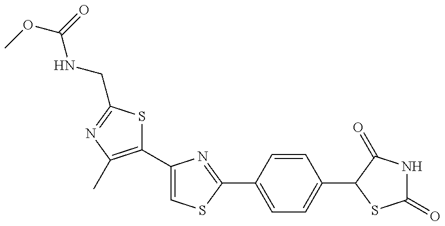 [SMILES: COC(=O)NCc1nc(C)c(-c2csc(-c3ccc(C4SC(=O)NC4=O)cc3)n2)s1] |
| 205 | 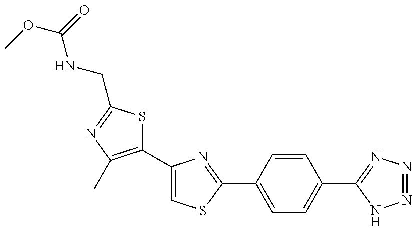 [SMILES: COC(=O)NCc1nc(C)c(-c2csc(-c3ccc(-c4nnn[nH]4)cc3)n2)s1] |
| 206 | 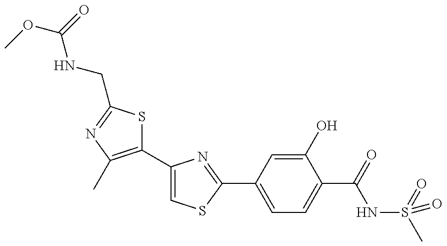 [SMILES: COC(=O)NCc1nc(C)c(-c2csc(-c3ccc(C(=O)NS(C)(=O)=O)c(O)c3)n2)s1] |
| 207 | 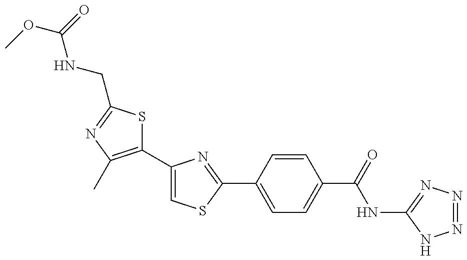 [SMILES: COC(=O)NCc1nc(C)c(-c2csc(-c3ccc(C(=O)Nc4nnn[nH]4)cc3)n2)s1] |
| 208 | 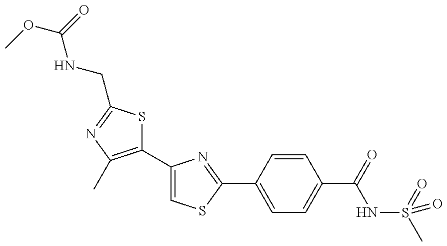 [SMILES: COC(=O)NCc1nc(C)c(-c2csc(-c3ccc(C(=O)NS(C)(=O)=O)cc3)n2)s1] |
| 209 | 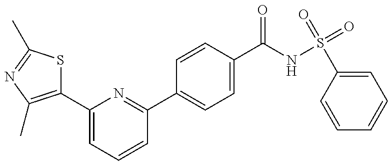 [SMILES: Cc1nc(C)c(-c2cccc(-c3ccc(C(=O)NS(=O)(=O)c4ccccc4)cc3)n2)s1] |
| 210 | 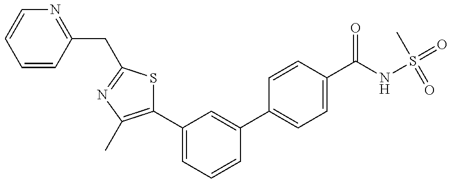 [SMILES: Cc1nc(Cc2ccccn2)sc1-c1cccc(-c2ccc(C(=O)NS(C)(=O)=O)cc2)c1] |
| 211 | 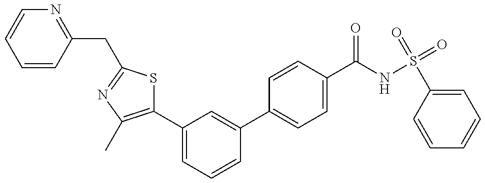 [SMILES: Cc1nc(Cc2ccccn2)sc1-c1cccc(-c2ccc(C(=O)NS(=O)(=O)c3ccccc3)cc2)c1] |
| 212 | 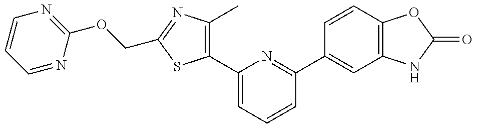 [SMILES: Cc1nc(COc2ncccn2)sc1-c1cccc(-c2ccc3oc(=O)[nH]c3c2)n1] |
| 213 | 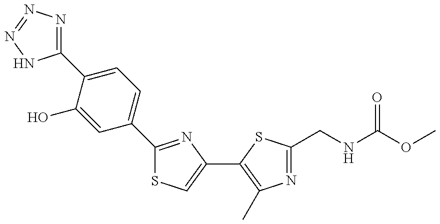 [SMILES: COC(=O)NCc1nc(C)c(-c2csc(-c3ccc(-c4nnn[nH]4)c(O)c3)n2)s1] |
| 214 | 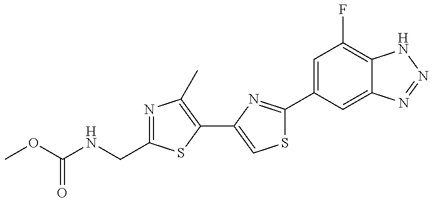 [SMILES: COC(=O)NCc1nc(C)c(-c2csc(-c3cc(F)c4[nH]nnc4c3)n2)s1] |
| 215 | 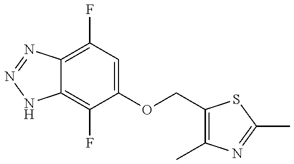 [SMILES: Cc1nc(C)c(COc2cc(F)c3nn[nH]c3c2F)s1] |
| 216 | 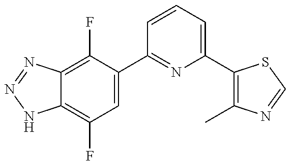 [SMILES: Cc1ncsc1-c1cccc(-c2cc(F)c3[nH]nnc3c2F)n1] |
| 217 | 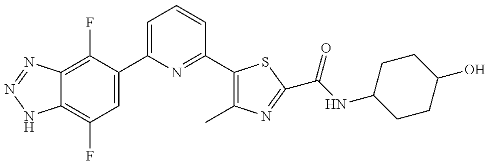 [SMILES: Cc1nc(C(=O)NC2CCC(O)CC2)sc1-c1cccc(-c2cc(F)c3[nH]nnc3c2F)n1] |
| 218 | 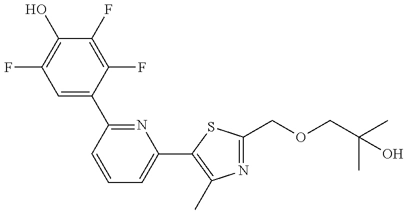 [SMILES: Cc1nc(COCC(C)(C)O)sc1-c1cccc(-c2cc(F)c(O)c(F)c2F)n1] |
| 219 | 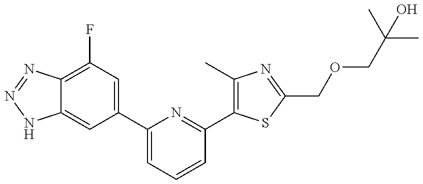 [SMILES: Cc1nc(COCC(C)(C)O)sc1-c1cccc(-c2cc(F)c3nn[nH]c3c2)n1] |
| 220 | 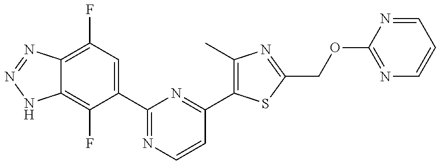 [SMILES: Cc1nc(COc2ncccn2)sc1-c1ccnc(-c2cc(F)c3nn[nH]c3c2F)n1] |
| 221 | 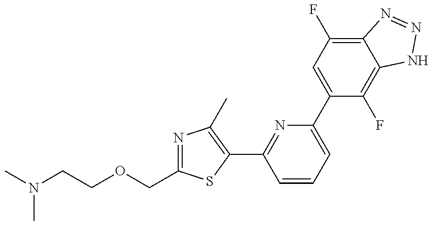 [SMILES: Cc1nc(COCCN(C)C)sc1-c1cccc(-c2cc(F)c3nn[nH]c3c2F)n1] |
| 222 | 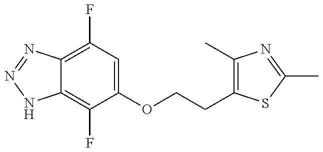 [SMILES: Cc1nc(C)c(CCOc2cc(F)c3nn[nH]c3c2F)s1] |
| 223 | 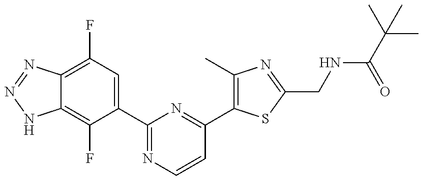 [SMILES: Cc1nc(CNC(=O)C(C)(C)C)sc1-c1ccnc(-c2cc(F)c3nn[nH]c3c2F)n1] |
| 224 | 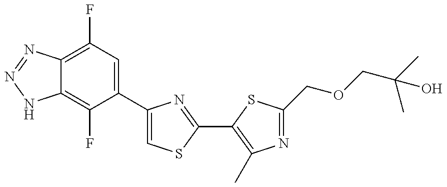 [SMILES: Cc1nc(COCC(C)(C)O)sc1-c1nc(-c2cc(F)c3nn[nH]c3c2F)cs1] |
| 225 | 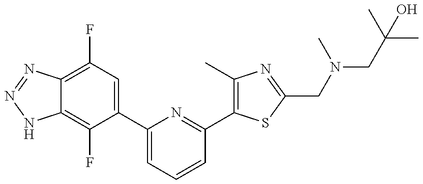 [SMILES: Cc1nc(CN(C)CC(C)(C)O)sc1-c1cccc(-c2cc(F)c3nn[nH]c3c2F)n1] |
| 226 | 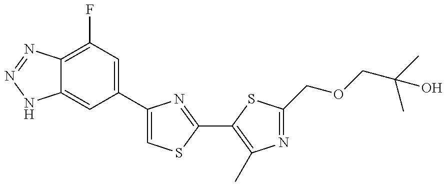 [SMILES: Cc1nc(COCC(C)(C)O)sc1-c1nc(-c2cc(F)c3nn[nH]c3c2)cs1] |
| 227 | 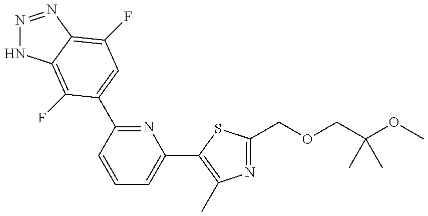 [SMILES: COC(C)(C)COCc1nc(C)c(-c2cccc(-c3cc(F)c4nn[nH]c4c3F)n2)s1] |
| 228 | 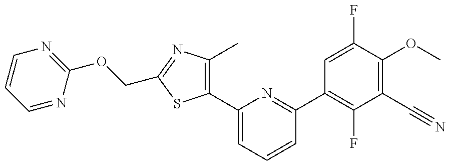 [SMILES: COc1c(F)cc(-c2cccc(-c3sc(COc4ncccn4)nc3C)n2)c(F)c1C#N] |
| 229 | 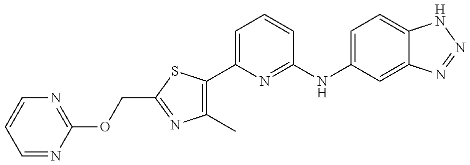 [SMILES: Cc1nc(COc2ncccn2)sc1-c1cccc(Nc2ccc3[nH]nnc3c2)n1] |
| 230 | 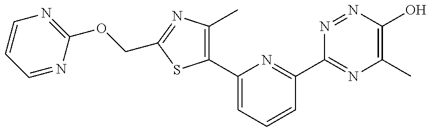 [SMILES: Cc1nc(-c2cccc(-c3sc(COc4ncccn4)nc3C)n2)nnc1O] |
| 231 | 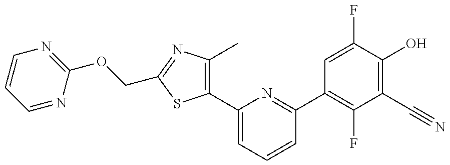 [SMILES: Cc1nc(COc2ncccn2)sc1-c1cccc(-c2cc(F)c(O)c(C#N)c2F)n1] |
| 232 | 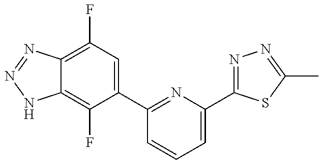 [SMILES: Cc1nnc(-c2cccc(-c3cc(F)c4nn[nH]c4c3F)n2)s1] |
| 233 | 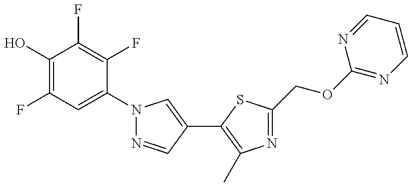 [SMILES: Cc1nc(COc2ncccn2)sc1-c1cnn(-c2cc(F)c(O)c(F)c2F)c1] |
| 234 | 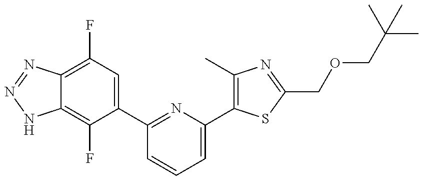 [SMILES: Cc1nc(COCC(C)(C)C)sc1-c1cccc(-c2cc(F)c3nn[nH]c3c2F)n1] |
| 235 | 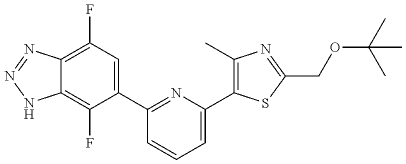 [SMILES: Cc1nc(COC(C)(C)C)sc1-c1cccc(-c2cc(F)c3nn[nH]c3c2F)n1] |
| 236 | 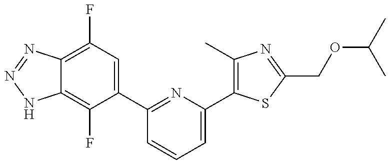 [SMILES: Cc1nc(COC(C)C)sc1-c1cccc(-c2cc(F)c3nn[nH]c3c2F)n1] |
| 237 | 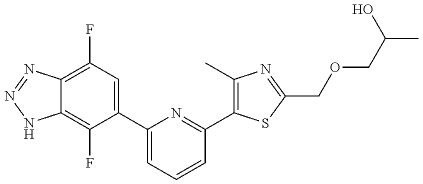 [SMILES: Cc1nc(COCC(C)O)sc1-c1cccc(-c2cc(F)c3nn[nH]c3c2F)n1] |
| 238 | 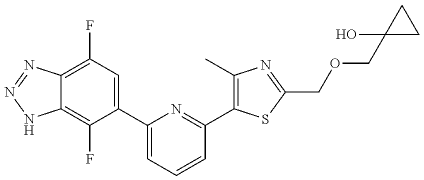 [SMILES: Cc1nc(COCC2(O)CC2)sc1-c1cccc(-c2cc(F)c3nn[nH]c3c2F)n1] |
| 239 | 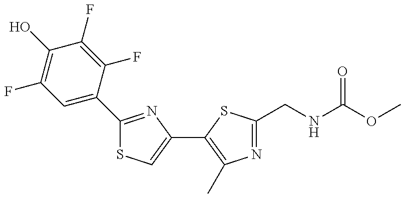 [SMILES: COC(=O)NCc1nc(C)c(-c2csc(-c3cc(F)c(O)c(F)c3F)n2)s1] |

or a pharmaceutically acceptable salt thereof.

### Methods of Synthesis

Compounds of Formula (I) or any variation thereof may be prepared according to the general reactions shown in Schemes IA, IB, II, and III

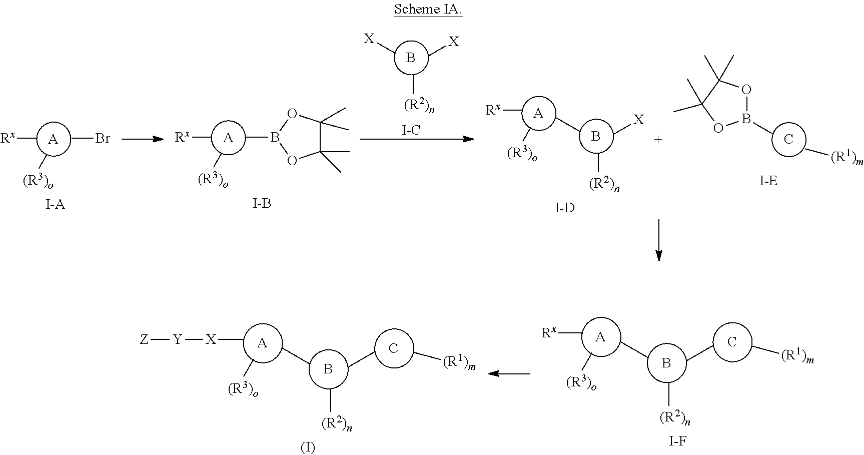

Scheme IA shows an exemplary route for preparing compounds of Formula (I), wherein R^{x} can be a group useful for conversion to a desired Z—Y—X group (such as shown in the non-limiting examples, Schemes IB, II, and III) and the remaining variables are as described herein. In some embodiments, compound I-A can be derivatized to form compound I-B, which can subsequently be coupled (e.g. under Suzuki coupling conditions) with an appropriate dihalide, compound I-C(wherein X is Cl or Br), to form compound I-D. Compound I-F can be achieved from compound I-D with an appropriate boronic acid ester, compound I-E, and metal catalyst. R^{x} can subsequently be derivatized to form the desired Z—Y—X group according to methods known in the art. In some embodiments, ring A, B, and C can be connected in any order (e.g., ring A and ring B, followed by ring C as shown in Scheme IA; or ring B and ring C, followed by ring A) according to similar methods as described herein.

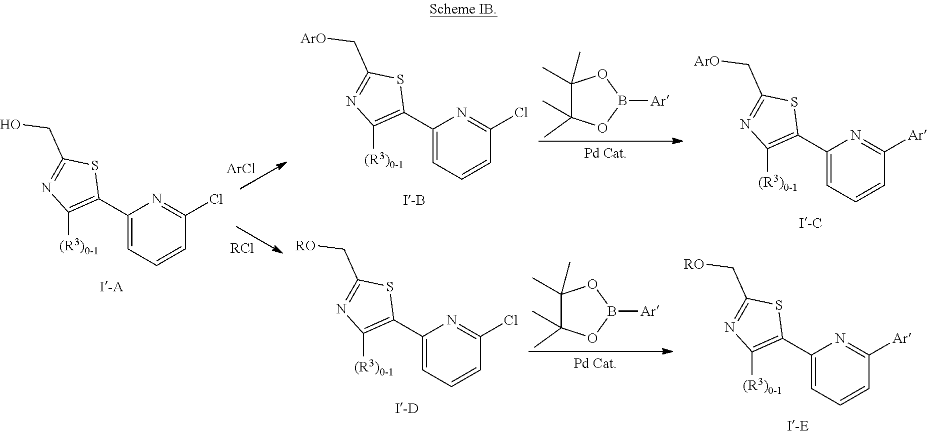

Scheme IB shows a route for preparing compounds of Formula (I) wherein Ring B is a heteroarylene group such as pyridinylene, Ar and Ar′ represent aryl groups, and R is a non-aryl group, such as an alkyl group. In some embodiments, esterification of the hydroxyl group of compound I′-A is achieved under standard conditions, such as in the presence of ArCl to form compound I′-B or in the presence of R-LG (e.g. R—Cl) to form compound I′-D. Suzuki coupling with the appropriate boronic ester in the presence of an appropriate palladium catalyst achieves compound I′-C from compound I′-B or compound I′-E from compound I′-D.

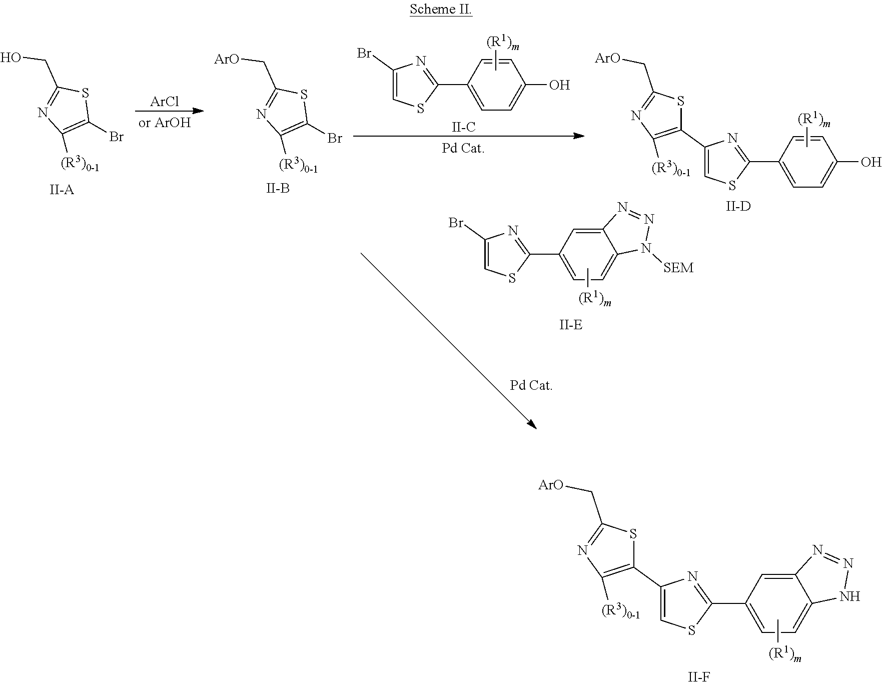

Scheme II shows a route for preparing compounds of Formula (I) wherein Rings A and B are each a heteroarylene group such as thiazolylene, Ring C is a phenylene or heteroarylene group, Ar represents an aryl group, and m, R³, and R¹ are as defined herein. In some embodiments, esterification of compound II-A in the presence of, for example, ArCl or ArOH, under standard conditions achieves compound II-B. Compound II-B may be combined with compound II-C in the presence of an appropriate boronic ester (such as 4,4,5,5-tetramethyl-2-(4,4,5,5-tetramethyl-1,3,2-dioxaborolan-2-yl)-1,3,2-dioxaborolane) and a palladium catalyst to provide compound II-D. Compound II-E can be achieved according to methods such as those described in Example S6. Compound II-B may also be combined with compound II-E in the presence of an appropriate boronic ester (such as 4,4,5,5-tetramethyl-2-(4,4,5,5-tetramethyl-1,3,2-dioxaborolan-2-yl)-1,3,2-dioxaborolane) and a palladium catalyst. Subsequent deprotection under standard conditions provides compound II-D.

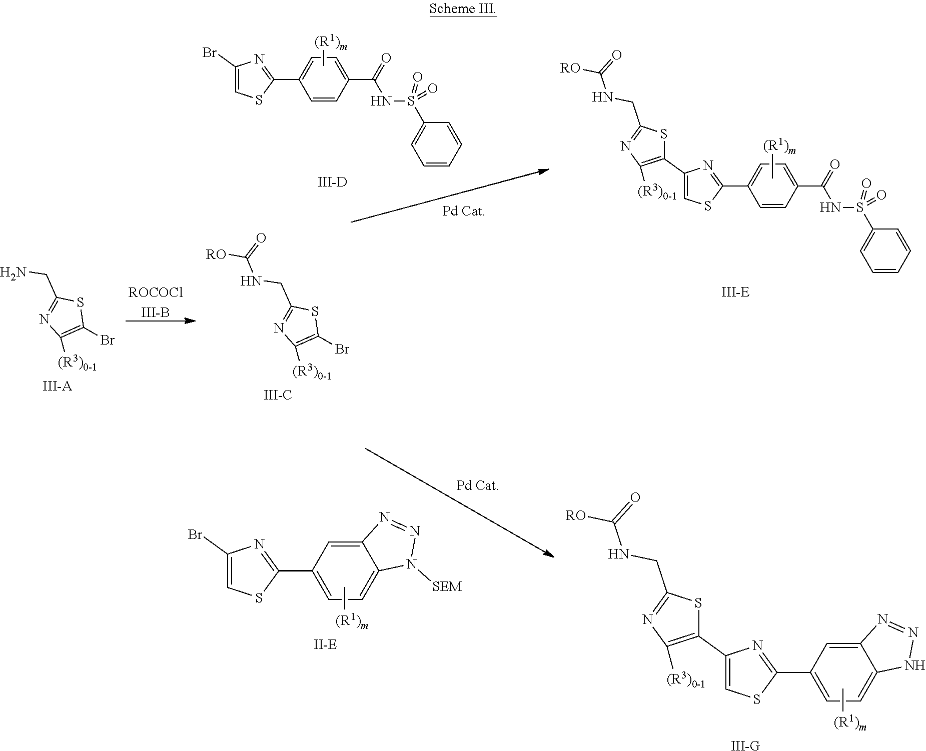

Scheme III shows a route for preparing compounds of Formula (I) wherein Rings A and B are each a heteroarylene group such as thiazolylene, Ring C is a phenylene or heteroarylene group, Ar represents an aryl group, R is a C₁₋₆ alkyl, and m, R³, and R¹ are as defined herein. In some embodiments, coupling of compound III-A and compound III-B under standard conditions achieves compound II-B. Compound III-C may be combined with compound III-D in the presence of an appropriate boronic ester (such as 4,4,5,5-tetramethyl-2-(4,4,5,5-tetramethyl-1,3,2-dioxaborolan-2-yl)-1,3,2-dioxaborolane) and a palladium catalyst to provide compound III-E. Compound III-C may also be combined with compound II-E in the presence of an appropriate boronic ester (such as 4,4,5,5-tetramethyl-2-(4,4,5,5-tetramethyl-1,3,2-dioxaborolan-2-yl)-1,3,2-dioxaborolane) and a palladium catalyst. Subsequent deprotection under standard conditions provides compound III-G.

It is understood that the synthetic processes disclosed herein may be modified to arrive at various compounds of the present disclosure by selection of appropriate reagents and starting materials. In some embodiments, the synthetic processes disclosed herein may be modified to arrive at compounds of Formula (I) having other substituents as those shown in the Schemes described herein by selection of appropriate reagents and starting materials.

All compounds of Formula (I) or any variation thereof as described herein which exist in free base or acid form can be converted to their pharmaceutically acceptable salts by treatment with the appropriate inorganic or organic base or acid by methods known to one skilled in the art. Salts of the compounds of the disclosure can be converted to their free base or acid form by standard techniques.

### Pharmaceutical Compositions and Formulations

In another aspect, provided herein are pharmaceutical compositions of any of the compounds detailed herein. Thus, the present disclosure includes pharmaceutical compositions comprising a compound disclosed herein, or a pharmaceutically acceptable salt thereof, and a pharmaceutically acceptable carrier or excipient. Pharmaceutical compositions according to the disclosure may take a form suitable for oral, buccal, parenteral, nasal, topical, or rectal administration, or a form suitable for administration by inhalation. Pharmaceutical compositions of the present disclosure comprise a compound of Formula (I), or a pharmaceutically acceptable salt thereof, and a pharmaceutically acceptable carrier, diluent, or excipient.

A compound described herein can be used in the preparation of a composition, such as a pharmaceutical composition, by combining the compound as an active ingredient with a pharmaceutically acceptable excipient. Some examples of materials which can serve as pharmaceutically acceptable excipients include: sugars, such as lactose, glucose and sucrose; starches, such as corn starch and potato starch; cellulose and its derivatives, such as sodium carboxymethyl cellulose, ethyl cellulose and cellulose acetate; surfactants, such as polysorbate 80 (i.e., Tween 80); powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Pharmaceutical formulations may be prepared by known pharmaceutical methods. Suitable formulations can be found in, for example, Remington: The Science and Practice of Pharmacy, Lippincott Williams & Wilkins, 21^{st} ed. (2005), which is incorporated herein by reference.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol and the like; and metal chelating agents, such as citric acid, ethylenediamine tetraacetic acid (EDTA), sorbitol, tartaric acid, phosphoric acid and the like.

Formulations of the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, this amount will range from about 1% to about 99% of active ingredient, or from about 5% to about 70%, or about 10% to about 30%.

In certain embodiments, a formulation of the present disclosure comprises an excipient selected from the group consisting of cyclodextrins, liposomes, micelle forming agents, e.g., bile acids and polymeric carriers, e.g., polyesters and polyanhydrides; and a compound of the present disclosure. In certain embodiments, an aforementioned formulation renders orally bioavailable a compound of the present disclosure.

Formulations of the disclosure suitable for oral administration may be in the form of capsules, cachets, pills, tablets, lozenges (using a flavored basis, usually sucrose and acacia or tragacanth), powders, granules or as a solution or a suspension in an aqueous or non-aqueous liquid or as an oil-in-water or water-in-oil liquid emulsion or as an elixir or syrup or as pastilles (using an inert base, such as gelatin and glycerin or sucrose and acacia) and/or as mouth washes and the like, each containing a predetermined amount of a compound of the present disclosure as an active ingredient. A compound of the present disclosure may also be administered as a bolus, electuary or paste.

In solid dosage forms of the disclosure for oral administration (capsules, tablets, pills, dragees, powders, granules and the like), the active ingredient is mixed with one or more pharmaceutically-acceptable carriers, such as sodium citrate or dicalcium phosphate and/or any of the following: fillers or extenders, such as starches, lactose, sucrose, glucose, mannitol and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates and sodium carbonate; solution retarding agents, such as paraffin; absorption accelerators, such as quaternary ammonium compounds; wetting agents, such as, for example, cetyl alcohol, glycerol monostearate and non-ionic surfactants; absorbents, such as kaolin and bentonite clay; lubricants, such a talc, calcium stearate, magnesium stearate, solid polyethylene glycols, sodium lauryl sulfate and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent.

The tablets and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter or by incorporating sterilizing agents in the form of sterile solid compositions that can be dissolved in sterile water or some other sterile injectable medium immediately before use. These compositions may also optionally contain opacifying agents and may be of a composition that they release the active ingredient(s) only or, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth and mixtures thereof.

Formulations of the pharmaceutical compositions of the disclosure for rectal or vaginal administration may be presented as a suppository, which may be prepared by mixing one or more compounds of the disclosure with one or more suitable nonirritating excipients or carriers comprising, for example, cocoa butter, polyethylene glycol, a suppository wax or a salicylate and which is solid at room temperature, but liquid at body temperature and, therefore, will melt in the rectum or vaginal cavity and release the active compound.

Dosage forms for the topical or transdermal administration of a compound of this disclosure include powders, sprays, ointments, pastes, creams, lotions, gels, solutions, patches and inhalants. The active compound may be mixed under sterile conditions with a pharmaceutically-acceptable carrier and with any preservatives, buffers or propellants which may be required.

The ointments, pastes, creams and gels may contain, in addition to an active compound of this disclosure, excipients, such as animal and vegetable fats, oils, waxes, paraffins, starch, tragacanth, cellulose derivatives, polyethylene glycols, silicones, bentonites, silicic acid, talc and zinc oxide or mixtures thereof.

Powders and sprays can contain, in addition to a compound of this disclosure, excipients such as lactose, talc, silicic acid, aluminum hydroxide, calcium silicates, and polyamide powder or mixtures of these substances. Sprays can additionally contain customary propellants, such as chlorofluorohydrocarbons and volatile unsubstituted hydrocarbons, such as butane and propane.

Pharmaceutical compositions of this disclosure suitable for parenteral administration comprise one or more compounds of the disclosure in combination with one or more pharmaceutically-acceptable sterile isotonic aqueous or nonaqueous solutions, dispersions, suspensions or emulsions or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the disclosure include water, ethanol, polyols (such as glycerol, propylene glycol, polyethylene glycol and the like) and suitable mixtures thereof, vegetable oils, such as olive oil and injectable organic esters, such as ethyl oleate. Proper fluidity can be maintained, for example, by the use of coating materials, such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenyl sorbic acid and the like. It may also be desirable to include isotonic agents, such as sugars, sodium chloride and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

### Methods of Use/Treatments

Compounds and compositions detailed herein, such as a pharmaceutical composition containing a compound of any formula provided herein, or a pharmaceutically acceptable salt thereof, and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

In one aspect, provided herein is a method of modulating bis-phosphoglycerate mutase (BPGM) comprising contacting either an effective amount of a compound described herein, or a pharmaceutically acceptable salt thereof, or an effective amount of a pharmaceutical composition provided herein, with the BPGM. In certain embodiments, the compounds of the present disclosure are allosteric modulators of BPGM that affect both the synthase and phosphatase functions of the enzyme.

In another aspect, provided herein is a method of treating sickle cell disease in a subject in need thereof, comprising administering to the subject a therapeutically effective amount of a compound described here, or a pharmaceutically acceptable salt thereof, or a therapeutically effective amount of a pharmaceutical composition described herein. In some embodiments, the sickle cell disease is HbSS, also referred to as sickle cell anemia. In some embodiments, the sickle cell disease is HbSC. In some embodiments, the sickle cell disease is HbS beta thalassemia. In some embodiments, the sickle cell disease is HbSD, HbSE, or HbSO.

In some embodiments, the subject is a mammal. In some embodiments, the subject is a primate, dog, cat, rabbit, or rodent. In some embodiments, the subject is a primate. In some embodiments, the subject is a human. In some embodiments, the human is at least about or is about any of 18, 21, 30, 50, 60, 65, 70, 80, or 85 years old. In some embodiments, the human is a child. In some embodiments, the human is less than about or about any of 21, 18, 15, 10, 5, 4, 3, 2, or 1 years old.

### Dosing and Method of Administration

The phrases “parenteral administration” and “administered parenterally” as used herein mean modes of administration other than enteral and topical administration, usually by injection and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the compounds of the present disclosure, or the pharmaceutical compositions of the present disclosure, are formulated into pharmaceutically acceptable dosage forms by conventional methods known to those of skill in the art.

Actual dosage levels of the active ingredients in the pharmaceutical compositions of this disclosure may be varied so as to obtain an amount of the active ingredient that is effective to achieve the desired therapeutic response for a particular patient, composition and mode of administration, without being toxic to the patient.

The selected dosage level will depend upon a variety of factors including the activity of the particular compound of the present disclosure employed or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated and like factors well known in the medical arts. A daily, weekly or monthly dosage (or other time interval) can be used.

A physician or veterinarian having ordinary skill in the art can readily determine and prescribe the effective amount of the pharmaceutical composition required. For example, the physician or veterinarian could start doses of the compounds of the disclosure employed in the pharmaceutical composition at levels lower than that required to achieve the desired therapeutic effect and then gradually increasing the dosage until the desired effect is achieved.

In general, a suitable daily dose of a compound of the disclosure will be that amount of the compound that is the lowest dose effective to produce a therapeutic effect (e.g., inhibit necrosis). Such an effective dose will generally depend upon the factors described above. Generally doses of the compounds of this disclosure for a patient, when used for the indicated effects, will range from about 0.0001 to about 100 mg per kg of body weight per day. In some embodiments, the daily dosage will range from 0.001 to 50 mg of compound per kg of body weight, and in some embodiments, from 0.01 to 10 mg of compound per kg of body weight.

If desired, the effective daily dose of the active compound may be administered as two, three, four, five, six or more sub-doses administered separately at appropriate intervals throughout the day, optionally, in unit dosage forms.

In certain embodiments, the present disclosure relates to compounds for modulating BPGM, wherein the compounds are represented by Formula (I). In certain embodiments, the compounds of the present disclosure are allosteric modulators of BPGM that affect both the synthase and phosphatase functions of the enzyme. In any event, the compounds of the present disclosure, in some embodiments, exert their effect on modulating BPGM at a concentration less than about 50 micromolar, or at a concentration less than about 10 micromolar or at a concentration less than 1 micromolar.

When the compounds of the present disclosure are administered as pharmaceuticals, to humans and animals, they can be given per se or as a pharmaceutical composition containing, for example, 0.1% to 99.5% (or, 0.5% to 90%) of active ingredient in combination with a pharmaceutically acceptable carrier.

The compounds of the present application or the compositions thereof may be administered once, twice, three or four times daily, using any suitable mode described above. Also, administration or treatment with the compounds may be continued for a number of days; for example, commonly treatment would continue for at least 7 days, 14 days or 28 days, for one cycle of treatment. Treatment cycles are well known and are frequently alternated with resting periods of about 1 to 28 days, commonly about 7 days or about 14 days, between cycles. The treatment cycles, in certain embodiments, may also be continuous.

When administered orally, the total daily dosage for a human subject may be between 1 mg and 1,000 mg, between about 1,000-2,000 mg/day, between about 10-500 mg/day, between about 50-300 mg/day, between about 75-200 mg/day or between about 100-150 mg/day.

The daily dosage may also be described as a total amount of a compound described herein administered per dose or per day. Daily dosage of a compound may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day or between about 15 to 150 mg/day.

In certain embodiments, the method comprises administering to the subject an initial daily dose of about 1 to 800 mg of a compound described herein and increasing the dose by increments until clinical efficacy is achieved. Increments of about 5, 10, 25, 50 or 100 mg can be used to increase the dose. The dosage can be increased daily, every other day, twice per week or once per week.

In certain embodiments, a compound or pharmaceutical preparation is administered orally. In certain embodiments, the compound or pharmaceutical preparation is administered intravenously. Alternative routes of administration include sublingual, intramuscular and transdermal administrations.

The preparations of the present disclosure may be given orally, parenterally, topically or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. In certain embodiments, the administration is oral.

### Kits/Articles of Manufacture

Also provided herein are kits that include a compound of the disclosure, or a pharmaceutically acceptable salt thereof, and suitable packaging. In certain embodiments, a kit further includes instructions for use. In one aspect, a kit includes a compound of the disclosure, or a pharmaceutically acceptable salt thereof, and a label and/or instructions for use of the compounds in the treatment of the indications, including the diseases or conditions, described herein.

Provided herein are also articles of manufacture that include a compound described herein, or a pharmaceutically acceptable salt thereof, in a suitable container. The container may be a vial, jar, ampoule, preloaded syringe and intravenous bag.

The kit can also contain instructions for using the compounds according to the disclosure. The kit can be compartmentalized to receive the containers in close confinement. As used herein, a kit such as a compartmentalized kit includes any kit in which compounds or agents are contained in separate containers. Illustrative examples of such containers include, but are not limited to, small glass containers, plastic containers or strips of plastic or paper. In some embodiments, the types of containers allow the skilled worker to efficiently transfer reagents from one compartment to another compartment such that the samples and reagents are not cross-contaminated and the agents or solutions of each container can be added in a quantitative fashion from one compartment to another. Such containers include, but are not limited to, a container that will accept a compound or combination of compounds and/or other agents of the disclosure. One or more compounds or agents can be provided as a powder (e.g. lyophilized powder) or precipitate. Such compound(s) can be resuspended prior to administration in a solution that may be provided as part of the kit or separately available. A kit can contain compounds or agents in other forms such as liquids, gels, solids, as described herein. Different compounds and/or agents may be provided in different forms in a single kit.

### EXAMPLES

The examples and preparations provided below further illustrate and exemplify the compounds of the present disclosure and methods for testing such compounds. It is to be understood that the scope of the present disclosure is not limited in any way by the scope of the following examples.

The chemical reactions in the Examples described can be readily adapted to prepare a number of other compounds disclosed herein, and alternative methods for preparing the compounds of this disclosure are deemed to be within the scope of this disclosure. For example, the synthesis of non-exemplified compounds according to the present disclosure can be performed by modifications apparent to those skilled in the art, for example by appropriately protecting interfering groups, by utilizing other suitable reagents known in the art other than those described, or by making routine modification of reaction conditions, reagents, and starting materials. Alternatively, other reactions disclosed herein or known in the art will be recognized as having applicability for preparing other compounds of the present disclosure.

The following abbreviations may be relevant for the application.

### Abbreviations

ACN: acetonitrile
Bu: butyl
DCM: dichloromethane
DEAD: diethyl azodicarboxylate
DIAD: diisopropyl azodicarboxylate
DIEA: N,N-diisopropylethylamine
DMF: N,N-dimethylformamide
DMSO: dimethylsulfoxide
ES-MS: electrospray ionization mass spectrometry
EtOAc or EA: ethylacetate
EtOH: ethanol
HPLC: high performance liquid chromatography
LCMS: liquid chromatography-mass spectrometry
Me: methyl
MeOH: methanol
NBS: N-bromosuccinimide
NMR: nuclear magnetic resonance
OAc: acetate
Pd(dppf)Cl₂·CH₂Cl₂: [1,1′-bis(diphenylphosphino)ferrocene]dichloropalladium(II)
PE: petroleum ether
PPh₃: triphenylphosphine
sat.: saturated
SEM-Cl: 2-(trimethylsilyl)ethoxymethyl chloride
TBAF: tetra-n-butylammonium fluoride
TBSCl: tert-butyldimethylsilyl chloride
TFA: trifluoroacetic acid
THF: tetrahydrofuran
TLC: thin layer chromatography

### SYNTHETIC EXAMPLES

### Example S1. Preparation of 4-fluoro-6-(4,4,5,5-tetramethyl-1,3,2-dioxaborolan-2-yl)-1-((2-(trimethylsilyl)ethoxy)methyl)-1H-benzo[d][1,2,3]triazole (1) (Key Intermediate A)

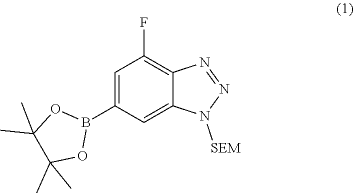 [SMILES: CC1(C)OB(c2cc(F)c3nnn(COCC[Si](C)(C)C)c3c2)OC1(C)C]

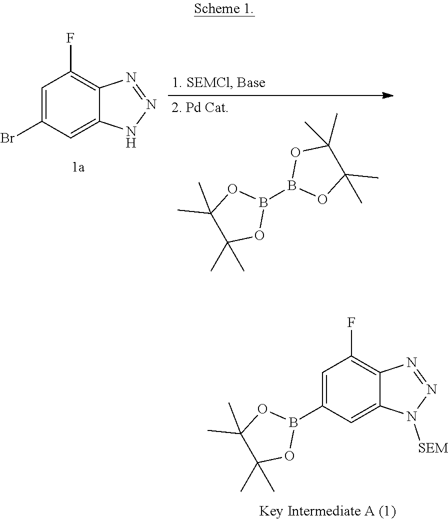

### Step 1: Synthesis of 6-bromo-4-fluoro-1-((2-(trimethylsilyl)ethoxy)methyl)-1H-benzo[d][1,2,3]triazole (1a)

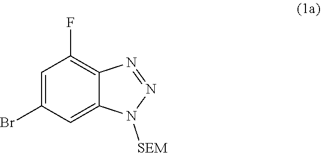 [SMILES: C[Si](C)(C)CCOCn1nnc2c(F)cc(Br)cc21]

To a solution of 5-bromo-7-fluoro-1H-benzotriazole (3 g, 12.50 mmol, 1 eq) in THF (100 mL) was added NaH (749.93 mg, 18.75 mmol, 60% purity, 1.5 eq). The mixture was stirred at 0° C. for 15 min. Then to the mixture was added SEM-Cl (3.13 g, 18.75 mmol, 3.32 mL, 1.5 eq). The mixture was stirred at 25° C. for 45 min. Water (40 mL) was added to the mixture. The mixture was extracted with ethyl acetate (40 mL×2). The combined organic phase was washed with brine (40 mL), dried over anhydrous Na₂SO₄, filtered and concentrated in vacuuo. The residue was purified with flash silica gel chromatography (ISCO®; 20 SepaFlash® Silica Flash Column, Eluent of 0-10% Petroleum/Ethyl acetate ether gradient @30 mL/min) to give 2-[(6-bromo-4-fluoro-benzotriazol-1-yl)methoxy]ethyl-trimethyl-silane 1a (1.4 g, 3.64 mmol, 58.2% yield, 90% purity) as a solid. ¹H NMR (400 MHz, CDCl₃) δ ppm 7.70 (d, J=1.2 Hz, 1H), 7.25 (dd, J=9.2, 1.2 Hz, 1H), 5.96 (s, 2H), 3.56-3.61 (m, 2H), 0.89-0.95 (m, 2H), −0.04 (s, 9H).

Step 2: Synthesis of 4-fluoro-6-(4,4,5,5-tetramethyl-1,3,2-dioxaborolan-2-yl)-1-((2-(trimethylsilyl)ethoxy)methyl)-1H-benzo[d][1,2,3]triazole (1). A mixture of 2-[(6-bromo-4-fluoro-benzotriazol-1-yl)methoxy]ethyl-trimethyl-silane 1a (600.00 mg, 1.56 mmol, 1 eq), potassium acetate (382.63 mg, 3.90 mmol, 2.5 eq), 4,4,5,5-tetramethyl-2-(4,4,5,5-tetramethyl-1,3,2-dioxaborolan-2-yl)-1,3,2-dioxaborolane (514.82 mg, 2.03 mmol, 1.3 eq) and cyclopentyl(diphenyl)phosphane;dichloromethane;dichloropalladium; iron (127.35 mg, 155.95 μmol, 0.1 eq) in dioxane (8 mL) was degassed and purged with N₂ for 3 times, and then the mixture was stirred at 100° C. for 12 h under N₂ atmosphere. Water (20 mL) was added to the mixture. The mixture was extracted with ethyl acetate (40 mL×2). The combined organic phase was washed with brine (40 mL), dried over anhydrous Na₂SO₄, filtered and concentrated in vacuo. The residue was purified with flash silica gel chromatography (ISCO®; 20 g SepaFlash® Silica Flash Column, Eluent of 0-30% Petroleum/Ethyl acetate ether gradient @ 30 mL/min) to give 2-[[4-fluoro-6-(4,4,5,5-tetramethyl-1,3,2-dioxaborolan-2-yl)benzotriazol-1-yl]methoxy]ethyl-trimethyl-silane 1 (800 mg, 1.42 mmol, 91.3% yield, 70% purity) as a solid. ¹HNMR (400 MHz, CDCl₃) δ ppm 7.96 (s, 1H), 7.46 (d, J=10.2 Hz, 1H), 6.01 (s, 2H), 3.58-3.64 (m, 2H), 1.39 (s, 12H), 0.89-0.95 (m, 2H), −0.05 (s, 9H).

### Example S2. Preparation of 4,7-difluoro-6-(4,4,5,5-tetramethyl-1,3,2-dioxaborolan-2-yl)-1H-benzotriazole (2) (Key Intermediate B)

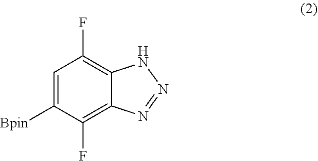

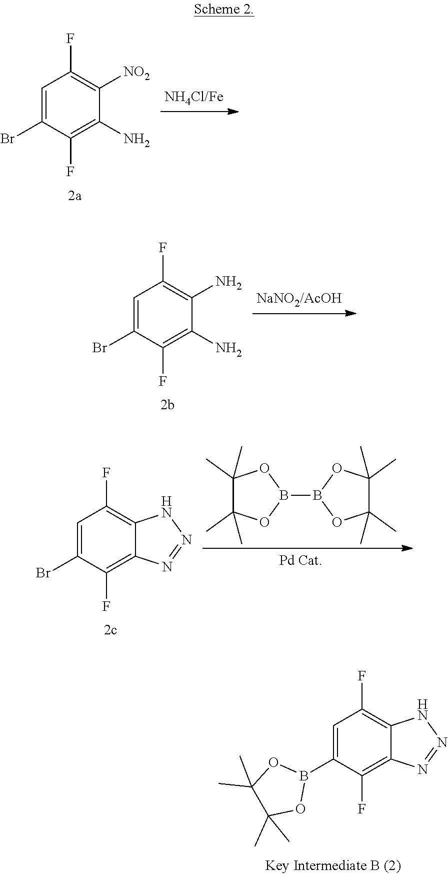

### Step 1: Synthesis of 3-bromo-2,5-difluoro-6-nitro-aniline (2a)

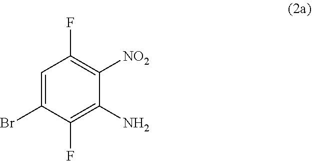 [SMILES: Nc1c(F)c(Br)cc(F)c1[N+](=O)[O-]]

To a solution of 1-bromo-2,5-difluoro-4-nitro-benzene (5 g, 21.01 mmol, 1 eq) in DMSO (100 mL) was added amino(trimethyl)ammonium iodide (5.09 g, 25.21 mmol, 1.2 eq), then t-BuOK (9.43 g, 84.04 mmol, 4 eq) was added in one portion at 15° C. After addition, the mixture was stirred at 15° C. for 15 min. TLC (PE/EtOAc=10/1) showed the reaction was completed. The reaction mixture was quenched with 10% HCl (30 mL) to make pH=4-5 at 10° C., then the mixture was poured into water (400 mL) and extracted with EtOAc (300 mL×3). The combined organic layers were washed with water (300 mL×3), brine (300 mL×3) and dried over Na₂SO₄, filtered and concentrated under reduced pressure. The residue was purified with flash silica gel chromatography (ISCO®; 80 g SepaFlash® Silica Flash Column, Eluent of 0-3.4% Ethyl acetate/Petroleum ether gradient @ 60 mL/min) to give 3-bromo-2,5-difluoro-6-nitro-aniline 2a (1.8 g, 6.40 mmol, 15.2% yield, 90% purity) as a solid. ¹H NMR (400 MHz, DMSO-d₆) δ ppm 7.21 (s, 2H), 7.00 (dd, J=5.6, 11.2 Hz, 1H).

### Step 2: Synthesis of 4-bromo-3,6-difluoro-benzene-1,2-diamine (2b)

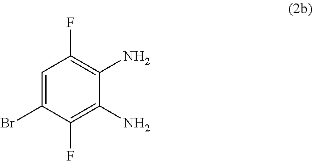 [SMILES: Nc1c(F)cc(Br)c(F)c1N]

To a solution of 3-bromo-2,5-difluoro-6-nitro-aniline 2a (2.6 g, 9.25 mmol, 1 eq) in EtOH (25 mL), THF (25 mL) and H₂O (15 mL) was added NH₄Cl (4.95 g, 92.49 mmol, 10 eq) and Fe power (5.17 g, 92.49 mmol, 10 eq). The reaction mixture was stirred at 55° C. for 2 h. TLC (PE/EtOAc=10/1) showed the reaction was completed. The reaction mixture was filtered, filtrate was dried over Na₂SO₄, filtered and concentrated to give crude 4-bromo-3,6-difluoro-benzene-1,2-diamine 2b (2 g, 8.07 mmol, 87.3% yield, 90% purity) as a solid. ¹H NMR (400 MHz, DMSO-d₆) δ ppm 6.64 (dd, J=6.0, 10.0 Hz, 1H), 5.05 (br.s, 2H), 4.89 (br.s, 2H).

### Step 3: Synthesis of 6-bromo-4,7-difluoro-1H-benzotriazole (2c)

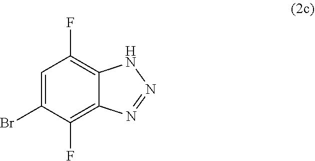 [SMILES: Fc1c(Br)cc(F)c2[nH]nnc12]

To a solution of 4-bromo-3,6-difluoro-benzene-1,2-diamine 2b (2 g, 8.97 mmol, 1 eq) in HOAc (15 mL) and H₂O (15 mL) was added HCl (12 M, 1.12 mL, 1.5 eq) and a solution of NaNO₂ (1.55 g, 22.42 mmol, 2.5 eq) in H₂O (15 mL) at 0° C. The reaction mixture was stirred at 0° C. for 30 min. TLC (PE/EtOAc=1/1) showed the reaction was completed. The reaction mixture was poured into water (30 mL) and extracted with EtOAc (30 mL×3). The combined organic layers were washed with sat. NaHCO₃ (30 mL×3) and dried over Na₂SO₄, filtered and concentrated under reduced pressure. The residue was purified with flash silica gel chromatography (ISCO®; 40 g SepaFlash® Silica Flash Column, Eluent of 0-10% Ethyl acetate/Petroleum ether gradient @ 40 mL/min) to give 6-bromo-4,7-difluoro-1H-benzotriazole 2c (1.6 g, 6.15 mmol, 68.6% yield, 90% purity) as a solid. ¹H NMR (400 MHz, CDCl₃) δ ppm 7.28 (dd, J=4.4, 8.4 Hz, 1H).

Step 4: Synthesis of 4,7-difluoro-6-(4,4,5,5-tetramethyl-1,3,2-dioxaborolan-2-yl)-1H-benzotriazole (2). A mixture of 6-bromo-4,7-difluoro-1H-benzotriazole 2c (1.5 g, 5.77 mmol, 1 eq), 4,4,5,5-tetramethyl-2-(4,4,5,5-tetramethyl-1,3,2-dioxaborolan-2-yl)-1,3,2-dioxaborolane (8.79 g, 34.62 mmol, 6 eq), Pd(dppf)Cl₂·CH₂Cl₂ (471.14 mg, 576.92 umol, 0.1 eq) and KOAc (4.53 g, 46.15 mmol, 8 eq) in DMSO (70 mL) was degassed and purged with N₂ for 3 times, and then the mixture was stirred at 100° C. for 12 h. The reaction mixture was poured into water (200 mL) and extracted with EtOAc (100 mL×3). The combined organic layers were washed with water (100 mL×3), brine (80 mL×3) and dried over Na₂SO₄, filtered and concentrated under reduced pressure. The residue was purified with flash silica gel chromatography (ISCO®; 40 g SepaFlash® Silica Flash Column, Eluent of 0-28% Ethyl acetate/Petroleum ether gradient @ 40 mL/min) to give 4,7-difluoro-6-(4,4,5,5-tetramethyl-1,3,2-dioxaborolan-2-yl)-1H-benzotriazole 2 (1.4 g, 3.74 mmol, 64.8% yield, 75% purity) as an oil. ¹H NMR (400 MHz, CDCl₃) δ ppm 7.37 (d, J=9.6 Hz, 1H), 1.38 (s, 12H).

### Example S3. Preparation of (5-bromo-4-methyl-thiazol-2-yl)methanamine (3) (Key Intermediate C)

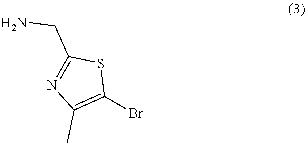

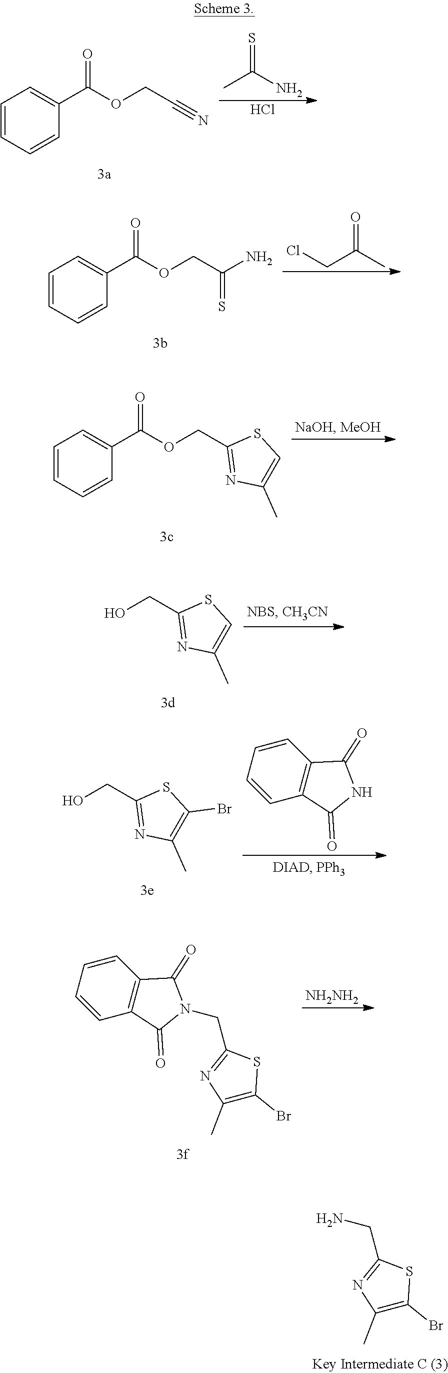

### Step 1: Synthesis of cyanomethyl benzoate (3a)

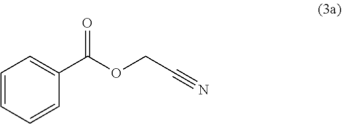 [SMILES: N#CCOC(=O)c1ccccc1]

To a suspension of benzoic acid (500 g, 4.09 mol, 625.00 mL, 1 eq) in DMF (1500 mL), K₂CO₃ (700.00 g, 5.06 mol, 1.24 eq) was added and the mixture was stirred until the evolution of gas has ceased. The reaction mixture was cooled to 0° C. and a solution of 2-bromoacetonitrile (491.10 g, 4.09 mol, 272.83 mL, 1 eq) in DMF (500 mL) was added drop-wise. The reaction mixture was stirred overnight (12 hr) at room temperature (25° C.). TLC (Petroleum ether/Ethyl acetate=3/1) indicated the material was consumed completely and one new spot formed. The reaction mixture was filtered and diluted with H₂O (3000 mL) and extracted with EA (2000 mL×2). The combined organic layers were washed with brine (2000 mL×2), dried over Na₂SO₄, filtered and concentrated under reduced pressure, the residue was purified by column chromatography (SiO₂, Petroleum ether/Ethyl acetate=10/1 to 5/1) to give cyanomethyl benzoate 3a (650 g, 4.03 mol, 98.51% yield) as an oil. ¹H NMR (400 MHz, CHLOROFORM-d) δ=8.11-8.03 (m, 2H), 7.67-7.60 (m, 1H), 7.53-7.45 (m, 2H), 4.97 (s, 2H).

### Step 2: Synthesis of (2-amino-2-thioxo-ethyl) benzoate (3b)

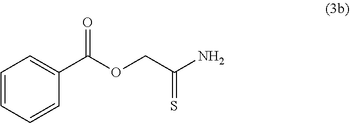 [SMILES: NC(=S)COC(=O)c1ccccc1]

Cyanomethyl benzoate 3a (170 g, 1.05 mol, 1 eq) and thioacetamide (118.88 g, 1.58 mol, 1.5 eq) were dissolved in DMF (800 mL). HCl/dioxane (4 M, 1.02 L, 3.87 eq) was added and the mixture was heated at 100° C. for 2 h. TLC (Petroleum ether/Ethyl acetate=1/1) indicated the material remained, and one major new spot with larger polarity was detected. The reaction mixture was reduced pressure to give a residue and then diluted with H₂O (1.2 L) and extracted with ethyl acetate (1.2 L×2). The combined organic layers were washed with brine (1.2 L×2), dried over Na₂SO₄, filtered and concentrated under reduced pressure and the crude product was triturated with Petroleum ether/Ethyl acetate=3/1 (300 mL) at 25° C. for 30 min to give (2-amino-2-thioxo-ethyl) benzoate 3b (130 g, crude) as a solid. ¹H NMR (400 MHz, DMSO-d₆) δ=9.95 (br s, 1H), 9.37 (br s, 1H), 8.13-8.04 (m, 2H), 7.72-7.64 (m, 1H), 7.60-7.49 (m, 2H), 4.97 (s, 2H).

### Step 3: Synthesis of (4-methylthiazol-2-yl)methyl benzoate (3c)

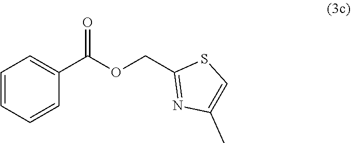 [SMILES: Cc1csc(COC(=O)c2ccccc2)n1]

To a solution of (2-amino-2-thioxo-ethyl) benzoate 3b (900 g, 4.61 mol, 1 eq) in EtOH (9000 mL) was added 1-chloropropan-2-one (639.77 g, 6.91 mol, 1.5 eq). The mixture was stirred at 90° C. for 12 hr. LCMS showed the material was consumed completely and one main peak with desired mass was detected. The reaction mixture was concentrated under reduced pressure to give a residue. The reaction mixture was diluted with H₂O (1000 mL) and extracted with Ethyl acetate (1000 mL×2). The combined organic layers were washed with brine (1000 mL×2), dried over Na₂SO₄, filtered and concentrated under reduced pressure to give (4-methylthiazol-2-yl)methyl benzoate 3c (1000 g, crude) as an oil. ¹H NMR (400 MHz, DMSO-d₆) δ=8.03-7.97 (m, 2H), 7.72-7.66 (m, 1H), 7.59-7.53 (m, 2H), 7.34 (d, J=1.0 Hz, 1H), 5.58 (s, 2H), 2.37 (d, J=0.9 Hz, 3H).

### Step 4: Synthesis of (4-methylthiazol-2-yl)methanol (3d)

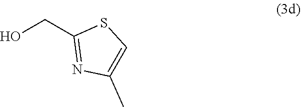

To a solution of (4-methylthiazol-2-yl)methyl benzoate 3c (250 g, 1.07 mol, 1 eq) in MeOH (1000 mL) was added NaOH (2 M, 1.25 L, 2.33 eq). The mixture was stirred at 25° C. for 2 hr. TLC (Petroleum ether/Ethyl acetate=0/1) indicated the material was consumed completely and one new spot formed. The reaction mixture was diluted with H₂O (300 mL) and extracted with EA (300 mL×2). The combined organic layers were washed with brine (300 mL×2), dried over Na₂SO₄, filtered and concentrated under reduced pressure to give (4-methylthiazol-2-yl)methanol 3d (340 g, crude) as an oil. ¹H NMR (400 MHz, DMSO-d₆) δ=7.13 (d, J=0.7 Hz, 1H), 5.96 (t, J=5.9 Hz, 1H), 4.67 (d, J=5.9 Hz, 2H), 2.32 (s, 3H).

### Step 5: Synthesis of (5-bromo-4-methyl-thiazol-2-yl)methanol (3e)

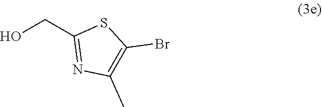

To a solution of (4-methylthiazol-2-yl)methanol 3d (100 g, 774.11 mmol, 1 eq) in MeCN (1000 mL) was added NBS (165.34 g, 928.94 mmol, 1.2 eq) at 0° C. The mixture was stirred at 25° C. for 0.5 h. LCMS showed the material was consumed completely and one main peak with desired mass was detected. The reaction mixture was diluted with H₂O (500 mL) and extracted with EA (500 mL×2). The combined organic layers were washed with brine (500 mL×2), dried over Na₂SO₄, filtered and concentrated under reduced pressure to give a residue and purified by column chromatography (SiO₂, Petroleum ether/Ethyl acetate=10/1 to 5/1) to give (5-bromo-4-methyl-thiazol-2-yl)methanol 3e (106 g, 509.43 mmol, 65.81% yield) as a solid. ¹H NMR (400 MHz, CHLOROFORM-d) δ=4.83 (s, 2H), 3.40-3.12 (m, 1H), 2.36 (s, 3H).

### Step 6: Synthesis of 2-[(5-bromo-4-methyl-thiazol-2-yl)methyl]isoindoline-1,3-dione (3f)

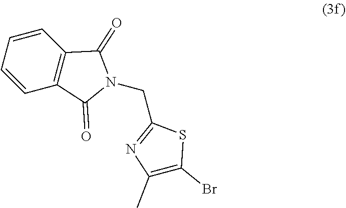 [SMILES: Cc1nc(CN2C(=O)c3ccccc3C2=O)sc1Br]

To a solution of (5-bromo-4-methyl-thiazol-2-yl)methanol 3e (3 g, 14.42 mmol, 1 eq), isoindoline-1,3-dione (2.76 g, 18.74 mmol, 1.3 eq) and triphenylphosphine (4.92 g, 18.74 mmol, 1.3 eq) in THF (40 mL). The mixture was stirred at 0° C. for 0.5 h. Then DIAD (3.79 g, 18.74 mmol, 3.64 mL, 1.3 eq) was added. The mixture was stirred at 25° C. for 1.5 h. After completion, the reaction mixture was poured into ammonium chloride saturated aqueous solution (50 mL) and extracted with ethyl acetate (20 mL×3). The combined organic layer was washed with brine (50 mL), dried over anhydrous sodium sulfate, filtered and concentrated. The residue was purified by flash silica gel chromatography (ISCO®; 40 g SepaFlash® Silica Flash Column, Eluent of 0-35% Ethyl acetate/Petroleum ether gradient @ 20 mL/min). The cut fraction was concentrated under reduced pressure to give 2-[(5-bromo-4-methyl-thiazol-2-yl)methyl]isoindoline-1,3-dione 3f (7.5 g, 10.45 mmol, 72.51% yield, 47% purity) as a solid.

Step 7: Synthesis of (5-bromo-4-methyl-thiazol-2-yl)methanamine (3). To a solution of 2-[(5-bromo-4-methyl-thiazol-2-yl)methyl]isoindoline-1,3-dione 3f (7.5 g, 10.45 mmol, 47% purity, 1 eq) in MeOH (150 mL) was added NH₂NH₂·H₂O (7.85 g, 156.81 mmol, 7.62 mL, 15 eq). The mixture was stirred at 25° C. for 16 h. After completion, the reaction mixture was filtered and concentrated under reduced pressure to give a residue. The reaction mixture was poured into 1N HCl (30 mL) to pH=3. Next, EA (100 mL) was added to the mixture. Then aqueous phase was added to the 2N NaOH (50 mL) and extracted with ethyl acetate (100 mL×3). The combined organic layer was washed with brine (50 mL), dried over anhydrous sodium sulfate, filtered and concentrated to give (5-bromo-4-methyl-thiazol-2-yl)methanamine 3 (2.5 g, crude) as a solid. ¹H NMR (400 MHz, DMSO-d₆) δ=3.90 (s, 2H), 2.33 (s, 2H), 2.26 (s, 3H).

### Example S4. Preparation of 4-(4-bromothiazol-2-yl)-2,3,6-trifluoro-phenol (4) (Key Intermediate D)

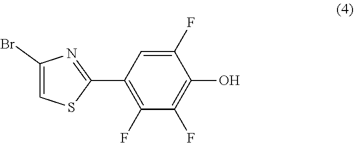 [SMILES: Oc1c(F)cc(-c2nc(Br)cs2)c(F)c1F]

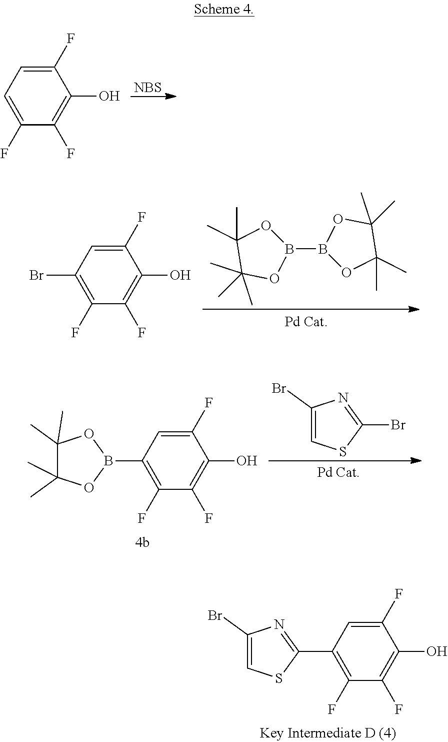

### Step 1: Synthesis of 4-bromo-2,3,6-trifluoro-phenol (4a)

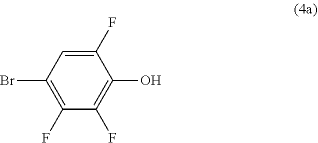 [SMILES: Oc1c(F)cc(Br)c(F)c1F]

A mixture of 2,3,6-trifluorophenol (20 g, 135.06 mmol, 1 eq), NBS (27.64 g, 155.32 mmol, 1.15 eq) in CHCl₃ (500 mL) was stirred at 0° C. for 2 h under N₂ atmosphere. The reaction mixture was concentrated under reduced pressure. The residue was purified by flash silica gel chromatography (ISCO®; 80 g SepaFlash® Silica Flash Column, Eluent of 0˜15% Ethyl acetate/Petroleum ether gradient @ 80 mL/min) to give 4-bromo-2,3,6-trifluoro-phenol 4a (30 g, 118.95 mmol, 88.1% yield, 90% purity) as a solid. ¹H NMR (400 MHz, DMSO-d₆) δ ppm 11.16 (s, 1H), 7.67-7.42 (m, 1H).

### Step 2: Synthesis of 2,3,6-trifluoro-4-(4,4,5,5-tetramethyl-1,3,2-dioxaborolan-2-yl)phenol (4b)

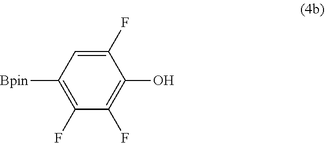

A mixture of 4-bromo-2,3,6-trifluoro-phenol 4a (10 g, 39.65 mmol, 1 eq), Pd(dppf)Cl₂ (1.45 g, 1.98 mmol, 0.05 eq), KOAc (9.73 g, 99.13 mmol, 2.5 eq), 4,4,5,5-tetramethyl-2-(4,4,5,5-tetramethyl-1,3,2-dioxaborolan-2-yl)-1,3,2-dioxaborolane (20.14 g, 79.30 mmol, 2 eq) in dioxane (250 mL) was degassed and purged with N₂ for 3 times, then the mixture was stirred at 80° C. for 12 h. The mixture was concentrated and then water (200 mL) was added then extracted with EtOAc (200 mL×3). The combined organic layers were washed with brine (200 mL), dried over Na₂SO₄, filtered and concentrated. The residue was purified with flash silica gel chromatography (ISCO®; 80 g SepaFlash® Silica Flash Column, Eluent of 0-20% Ethyl acetate/Petroleum ether gradient @ 60 mL/min) to give 2,3,6-trifluoro-4-(4,4,5,5-tetramethyl-1,3,2-dioxaborolan-2-yl)phenol 4b (4 g, 13.14 mmol, 33.1% yield, 90% purity) as a solid. ¹H NMR (400 MHz, DMSO-d₆) δ ppm 11.23 (s, 1H), 7.05-6.96 (m, 1H), 1.24 (s, 12H).

Step 3: Synthesis of 4-(4-bromothiazol-2-yl)-2,3,6-trifluoro-phenol (4). A mixture of 2,3,6-trifluoro-4-(4,4,5,5-tetramethyl-1,3,2-dioxaborolan-2-yl)phenol 4b (0.5 g, 1.64 mmol, 1 eq), Pd(dppf)Cl₂ (120.15 mg, 164.21 μmol, 0.1 eq), K₃PO₄.3H₂O (1.31 g, 4.93 mmol, 3 eq), 2,4-dibromothiazole (1.20 g, 4.93 mmol, 3 eq) in dioxane (8 mL) and H₂O (2 mL) was degassed and purged with N₂ for 3 times, and then the mixture was stirred at 60° C. for 0.5 h under microwave (2 Bar). TLC (PE/EtOAc=5/1) indicated the reaction was completed. The mixture was concentrated and water (200 mL) was added. The mixture was extracted with EtOAc (200 mL×3). The combined organic layers were washed with brine (200 mL), dried over Na₂SO₄, filtered and concentrated. The residue was purified with flash silica gel chromatography (ISCO®; 80 g SepaFlash® Silica Flash Column, Eluent of 0-30% Ethyl acetate/Petroleum ether gradient @ 60 mL/min) to give 4-(4-bromothiazol-2-yl)-2,3,6-trifluoro-phenol 4 (0.28 g, 812.67 μmol, 49.5% yield, 90% purity) as a solid. ¹H NMR (400 MHz, DMSO-d₆) δ ppm 11.72 (br s, 1H), 8.05 (s, 1H), 7.74-7.71 (m, 1H).

### Example S5. Preparation of 5-(6-chloro-2-pyridyl)-4-methyl-2-(pyrimidin-2-yloxymethyl)thiazole (5) (Key Intermediate E)

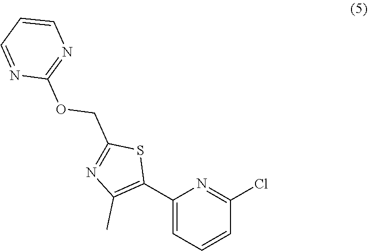 [SMILES: Cc1nc(COc2ncccn2)sc1-c1cccc(Cl)n1]

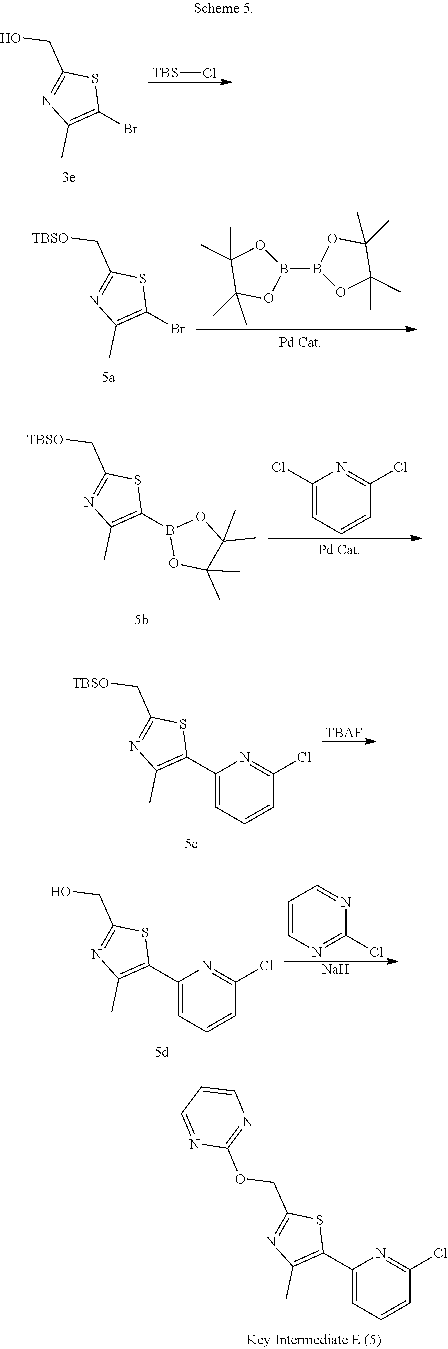

### Step 1: Synthesis of 5-bromo-2-(((tert-butyldimethylsilyl)oxy)methyl)-4-methylthiazole (5a)

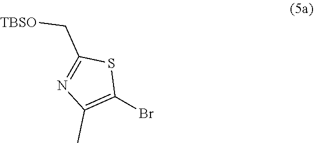 [SMILES: Cc1nc(CO[Si](C)(C)C(C)(C)C)sc1Br]

To a solution of (5-bromo-4-methyl-thiazol-2-yl)methanol 3e (40 g, 173.01 mmol, 1 eq) in anhydrous DCM (150 mL) was added TBSCl (33.90 g, 224.92 mmol, 27.56 mL, 1.3 eq) and imidazole (17.67 g, 259.52 mmol, 1.5 eq) at 0° C. The mixture was warmed to 25° C. and stirred for 12 h. The reaction mixture was concentrated under reduced pressure to remove DCM. The residue was diluted with H₂O (100 mL) and extracted with EtOAc (150 mL×3). The combined organic layers were washed with brine (100 mL×3), dried over anhydrous Na₂SO₄, filtered and concentrated under reduced pressure to give a residue. The residue was purified by flash silica gel chromatography (ISCO®; 40 g SepaFlash® Silica Flash Column, Eluent of 0-3% Ethyl acetate/Petroleum ether gradient @ 60 mL/min) to give (5-bromo-4-methyl-thiazol-2-yl)methoxy-tert-butyl-dimethyl-silane 5a (57 g, 159.15 mmol, 92.0% yield, 90% purity) as an oil. ¹H NMR (400 MHz, CDCl₃) δ ppm 4.85 (s, 2H), 2.36 (s, 3H), 0.95 (s, 9H), 0.13 (s, 6H).

### Step 2: Synthesis of 2-(((tert-butyldimethylsilyl)oxy)methyl)-4-methyl-5-(4,4,5,5-tetramethyl-1,3,2-dioxaborolan-2-yl)thiazole (5b)

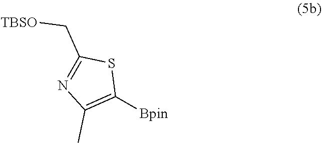

To a solution of (5-bromo-4-methyl-thiazol-2-yl)methoxy-tert-butyl-dimethyl-silane 5a (10 g, 27.92 mmol, 1 eq) in anhydrous THF (200 mL) was added n-BuLi (2.5 M, 16.75 mL, 1.5 eq) dropwise at −78° C. under N₂. The reaction mixture was stirred at −78° C. for 0.5 h. 4,4,5,5-tetramethyl-1,3,2-dioxaborolane (8.93 g, 69.80 mmol, 10.13 mL, 2.5 eq) was added to the mixture in dropwise at −78° C. for 0.5 h. After addition, the reaction was warmed to 25° C. and stirred for another 12 h. The reaction was quenched with saturated NH₄Cl (100 mL) at 0° C. and extracted with EtOAc (200 mL×3) and washed with brine (500 mL×3). The combined organic layers were dried over anhydrous Na₂SO₄, filtered and concentrated to give crude residue. The residue was purified by flash silica gel chromatography (ISCO®; 80 g SepaFlash® Silica Flash Column, Eluent of 0-10% Ethyl acetate/Petroleum ether gradient @ 60 mL/min) to give 2-(((tert-butyldimethylsilyl)oxy)methyl)-4-methyl-5-(4,4,5,5-tetramethyl-1,3,2-dioxaborolan-2-yl)thiazole 5b (6 g, 14.62 mmol, 52.4% yield, 90% purity) as an oil. ¹H NMR (400 MHz, CDCl₃) δ ppm 4.95 (s, 2H), 2.60 (s, 3H), 1.34 (s, 12H), 0.96 (s, 9H), 0.13 (s, 6H).

### Step 3: Synthesis of 2-(((tert-butyldimethylsilyl)oxy)methyl)-5-(6-chloropyridin-2-yl)-4-methylthiazole (5c)

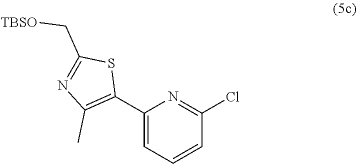 [SMILES: Cc1nc(CO[Si](C)(C)C(C)(C)C)sc1-c1cccc(Cl)n1]

To a solution of 2,6-dichloropyridine (2.34 g, 15.84 mmol, 1.3 eq) in dioxane (120 mL), H₂O (12 mL) was added 2-(((tert-butyldimethylsilyl)oxy)methyl)-4-methyl-5-(4,4,5,5-tetramethyl-1,3,2-dioxaborolan-2-yl)thiazole 5b (5 g, 12.18 mmol, 1 eq), Cs₂CO₃ (11.91 g, 36.55 mmol, 3 eq) and Pd(dppf)Cl₂ (534.81 mg, 730.91 μmol, 0.06 eq). The mixture was stirred at 90° C. for 2 h under N₂ atmosphere. The reaction was diluted with water (100 mL) and extracted with EtOAc (80 mL×3). The combined organic layers were washed with brine (100 mL), dried over anhydrous Na₂SO₄, filtered and concentrated under reduced pressure. The residue was purified by flash silica gel chromatography (ISCO®; 20 g SepaFlash® Silica Flash Column, Eluent of 0-10% Ethylacetate/Petroleum ethergradient @ 35 mL/min) to give 2-(((tert-butyldimethylsilyl)oxy)methyl)-5-(6-chloropyridin-2-yl)-4-methylthiazole 5c (1.9 g, 4.82 mmol, 39.5% yield, 90% purity) as an oil. ¹H NMR (400 MHz, CDCl₃) δ ppm 7.68 (t, J=7.6 Hz, 1H), 7.44 (d, J=7.6 Hz, 1H), 7.21 (d, J=7.6 Hz, 1H), 4.94 (s, 2H), 2.67 (s, 3H), 0.97 (s, 9H), 0.16 (s, 6H).

### Step 4: Synthesis of [5-(6-chloro-2-pyridyl)-4-methyl-thiazol-2-yl]methanol (5d)

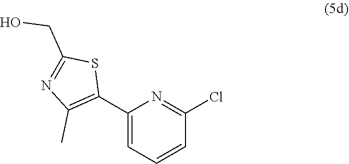

To a solution of 2-(((tert-butyldimethylsilyl)oxy)methyl)-5-(6-chloropyridin-2-yl)-4-methylthiazole 5c (1.9 g, 4.82 mmol, 1 eq) in THF (15 mL) was added TBAF (1 M, 5.78 mL, 1.2 eq). The mixture was stirred at 25° C. for 1 h under N₂ atmosphere. TLC (EtOAc) indicated the starting material was consumed. The reaction mixture was concentrated under reduced pressure to give residue. The residue was purified by flash silica gel chromatography (ISCO®; 12 g SepaFlash® Silica Flash Column, Eluent of 100% Ethyl acetate gradient @ 30 mL/min) to give [5-(6-chloro-2-pyridyl)-4-methyl-thiazol-2-yl]methanol 5d (1 g, 3.74 mmol, 77.6% yield, 90% purity) as a solid. ¹H NMR (400 MHz, CDCl₃) δ ppm 7.70 (t, J=7.6 Hz, 1H), 7.45 (d, J=7.6 Hz, 1H), 7.23 (d, J=8.0 Hz, 1H), 4.93 (d, J=6.0 Hz, 2H), 2.69 (s, 3H).

Step 5: Synthesis of 5-(6-chloro-2-pyridyl)-4-methyl-2-(pyrimidin-2-yloxymethyl)thiazole (5). To a solution of [5-(6-chloro-2-pyridyl)-4-methyl-thiazol-2-yl]methanol 5d (1 g, 3.74 mmol, 1 eq) in anhydrous DMF (10 mL) was added NaH (164.50 mg, 4.11 mmol, 60% purity, 1.1 eq). The mixture was stirred at 0° C. for 15 min. Then 2-chloropyrimidine (1.28 g, 11.22 mmol, 3 eq) was added to the mixture at 0° C. After addition, the mixture was stirred at 25° C. for 45 min. TLC (Petroleum ether/EtOAc=1:1) indicated one major new spot formed. Water (40 mL) was added and extracted with ethyl acetate (40 mL×2). The combined organic phase was washed with brine (40 mL), dried with anhydrous Na₂SO₄, filtered and concentrated under reduced pressure. The residue was purified by flash silica gel chromatography (ISCO®; 12 g SepaFlash® Silica Flash Column, Eluent of 0˜70% EtOAc/PE ethergradient @30 mL/min) to give 5-(6-chloro-2-pyridyl)-4-methyl-2-(pyrimidin-2-yloxymethyl)thiazole 5 (700 mg, 1.98 mmol, 52.9% yield, 90% purity) as a solid. ¹H NMR (400 MHz, CDCl₃) δ ppm 8.58 (d, J=4.8 Hz, 2H), 7.68 (t, J=8.0 Hz, 1H), 7.44 (d, J=7.6 Hz, 1H), 7.22 (d, J=7.6 Hz, 1H), 7.02 (t, J=4.8 Hz, 1H), 5.71 (s, 2H), 2.71 (s, 3H).

### Example S6. Preparation of 2-[[5-(4-bromothiazol-2-yl)-4,7-difluoro-benzotriazol-2-yl]methoxy]ethyl-trimethyl-silane (6) (Key Intermediate F)

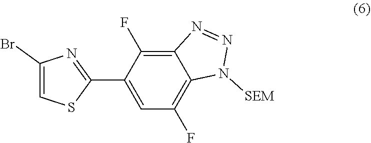 [SMILES: C[Si](C)(C)CCOCn1nnc2c(F)c(-c3nc(Br)cs3)cc(F)c21]

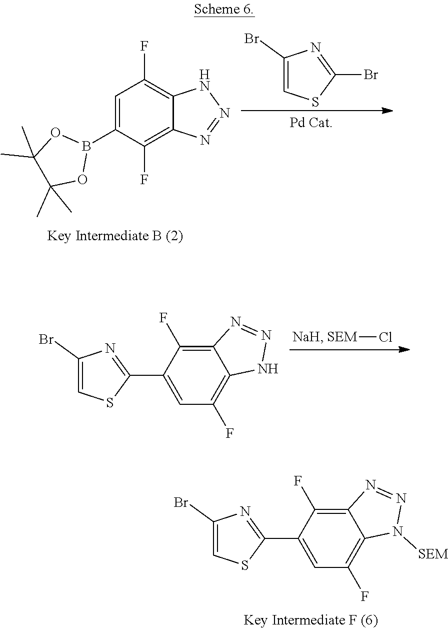 [SMILES: CC1(C)OB(c2cc(F)c3[nH]nnc3c2F)OC1(C)C ; Brc1csc(Br)n1 ; Fc1c(-c2nc(Br)cs2)cc(F)c2[nH]nnc12 ; C[Si](C)(C)CCOCCl ; C[Si](C)(C)CCOCn1nnc2c(F)c(-c3nc(Br)cs3)cc(F)c21]

### Step 1: Synthesis of 4-bromo-2-(4,7-difluoro-1H-benzotriazol-5-yl)thiazole (6a)

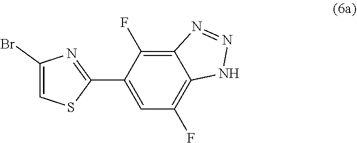 [SMILES: Fc1c(-c2nc(Br)cs2)cc(F)c2[nH]nnc12]

A mixture of 4,7-difluoro-5-(4,4,5,5-tetramethyl-1,3,2-dioxaborolan-2-yl)-1H-benzotriazole Key Intermediate B (2) (8 g, 28.46 mmol, 1 eq), 2,4-dibromothiazole (13.83 g, 56.93 mmol, 2 eq), Na₂CO₃ (9.05 g, 85.39 mmol, 3 eq) and Pd(dppf)Cl₂·CH₂Cl₂ (2.32 g, 2.85 mmol, 0.1 eq) in dioxane (100 mL) and H₂O (25 mL) was degassed and charged with N₂ three times. The reaction mixture was stirred at 80° C. for 12 h under N₂. LCMS showed 22% of desired was observed. Ice-water (300 mL) was added, acidified by 1N HCl (˜180 mL) to pH=5, extracted with EtOAc (500 mL×2), washed with brine (300 mL), dried over anhydrous Na₂SO₄, filtered and concentrated to give the crude. Then purified by reversed-phase HPLC (0.1% FA condition) to give 4-bromo-2-(4,7-difluoro-1H-benzotriazol-5-yl)thiazole 6a (1.3 g, 4.10 mmol, 14.40% yield) as a solid. ¹H NMR (400 MHz, DMSO-d6) δ=8.13 (s, 1H), 7.95-7.90 (m, 1H).

Step 2: Synthesis of 2-[[5-(4-bromothiazol-2-yl)-4,7-difluoro-benzotriazol-2-yl]methoxy]ethyl-trimethyl-silane (6). To a suspension of NaH (416.22 mg, 10.41 mmol, 60% purity, 3 eq) in THF (7 mL) was added a solution of 4-bromo-2-(4,7-difluoro-1H-benzotriazol-5-yl)thiazole 6a (1.1 g, 3.47 mmol, 1 eq) in THF (7 mL) at 0° C. under N₂ atmosphere. The mixture was stirred at 0° C. for 30 min, then SEM-Cl (751.82 mg, 4.51 mmol, 798.11 uL, 1.3 eq) was added. The resulting mixture was stirred at 0° C. under N₂ atmosphere for 30 min. TLC showed material was consumed and some new spots formed. The reaction mixture was quenched with sat. NH₄Cl (20 mL) solution and extracted with ethyl acetate (20 mL×2), the organic layer was dried over Na₂SO₄, filtered, filtrate was concentrated under reduced pressure. The residue was purified with flash silica gel chromatography (Eluent of 0-10% Ethyl acetate/Petroleum ether) to give 2-[[5-(4-bromothiazol-2-yl)-4,7-difluoro-benzotriazol-2-yl]methoxy]ethyl-trimethyl-silane 6 (550 mg, 1.23 mmol, 35.44% yield) as a solid. ¹H NMR (400 MHz, CHLOROFORM-d) δ=8.06 (dd, J=4.6, 10.6 Hz, 1H), 7.44 (s, 1H), 6.03 (s, 2H), 3.84-3.77 (m, 2H), 1.03-0.97 (m, 2H), 0.00 (s, 9H).

### Example S7. Preparation of 5-[2-(7-fluoro-1H-benzotriazol-5-yl)thiazol-4-yl]-4-methyl-2-(pyridazin-3-yloxymethyl)thiazole (7)

General Scheme for Compounds 7, 10 and 11.

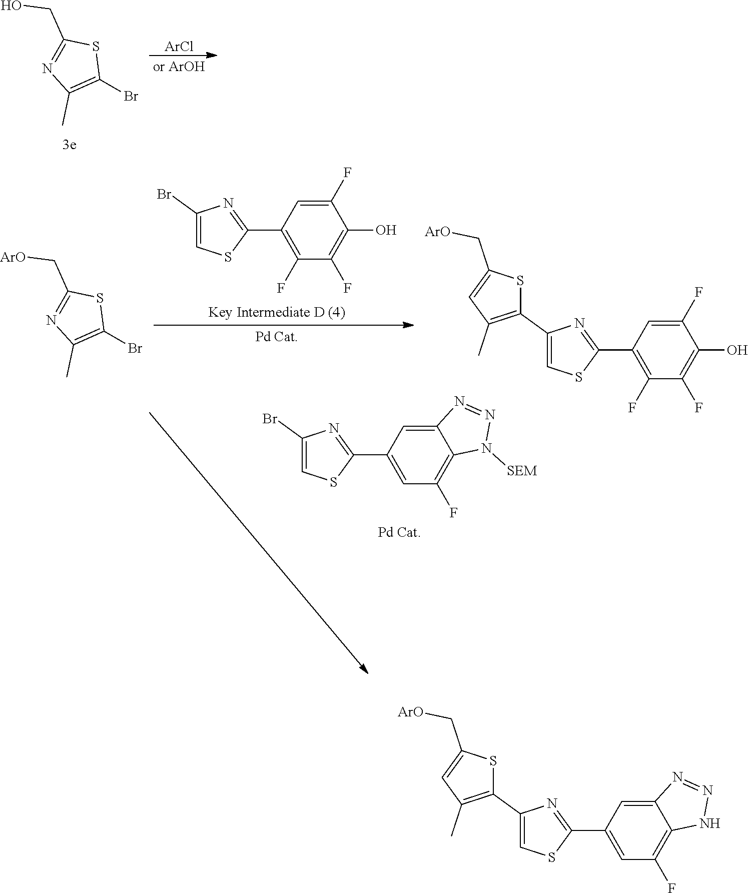

### Step 1: Synthesis of 5-bromo-4-methyl-2-(pyridazin-3-yloxymethyl)thiazole (7a)

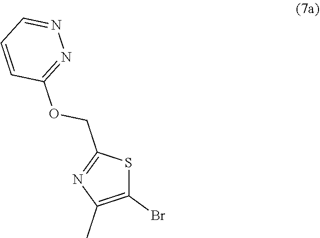

To a mixture of (5-bromo-4-methyl-thiazol-2-yl)methanol 3e (0.6 g, 2.60 mmol, 1 eq) in THF (15 mL) was added NaH (311.39 mg, 7.79 mmol, 60% purity, 3 eq) and purged with N₂ for 3 times, and then the mixture was stirred at 20° C. for 0.5 h. 3-Chloropyridazine (356.68 mg, 3.11 mmol, 1.2 eq) was added to the reaction mixture, the mixture was stirred at 20° C. for 12 h. The reaction mixture was quenched with addition of water (50 mL) at 0° C. The mixture was extracted with EtOAc (50 mL×3). The combined organic layers were washed with brine (50 mL), dried over Na₂SO₄, filtered and concentrated. The residue was purified with flash silica gel chromatography (ISCO®; 20 g SepaFlash® Silica Flash Column, Eluent of 0˜70% Ethylacetate/Petroleum ether gradient @ 40 mL/min) to give 5-bromo-4-methyl-2-(pyridazin-3-yloxymethyl)thiazole 7a (110 mg, 345.97 μmol, 13.3% yield, 90% purity) as a solid. ¹H NMR (400 MHz, DMSO-d₆) δ ppm 8.97 (d, J=4.4 Hz, 1H), 7.71 (dd, J=4.4, 9.0 Hz, 1H), 7.36 (dd, J=1.4, 8.8 Hz, 1H), 5.74 (s, 2H), 2.34 (s, 3H).

### Step 2: Synthesis of 2-[[5-(4-bromothiazol-2-yl)-7-fluoro-benzotriazol-1-yl]methoxy]ethyl-trimethyl-silane (7b)

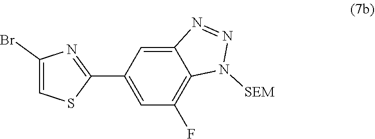 [SMILES: C[Si](C)(C)CCOCn1nnc2cc(-c3nc(Br)cs3)cc(F)c21]

A mixture of 2-[[7-fluoro-5-(4,4,5,5-tetramethyl-1,3,2-dioxaborolan-2-yl)benzotriazol-1-yl]methoxy]ethyl-trimethyl-silane Key Intermediate A (1) (0.6 g, 1.22 mmol, 1 eq), Pd(dppf)Cl₂ (89.29 mg, 122.03 μmol, 0.1 eq), K₃PO₄·3H₂O (974.96 mg, 3.66 mmol, 3 eq), 2,4-dibromothiazole (741.11 mg, 3.05 mmol, 2.5 eq) in dioxane (8 mL) and H₂O (2 mL) was degassed and purged with N₂ for 3 times, and then the mixture was stirred at 60° C. for 0.5 h under microwave (1bar). The mixture was concentrated and then water (100 mL) was added. The mixture was extracted with EtOAc (80 mL×3). The combined organic layers were washed with brine (80 mL), dried over Na₂SO₄, filtered and concentrated. The residue was purified with flash silica gel chromatography (ISCO®; 24 g SepaFlash® Silica Flash Column, Eluent of 0-30% Ethylacetate/Petroleum ether gradient @ 40 mL/min) to give 2-[[5-(4-bromothiazol-2-yl)-7-fluoro-benzotriazol-1-yl]methoxy]ethyl-trimethyl-silane 7b (0.51 g, 1.07 mmol, 87.6% yield, 90% purity) as a solid. ¹HNMR (400 MHz, DMSO-d₆) δ ppm 8.55 (s, 1H), 8.02 (s, 2H), 6.10 (s, 2H), 3.62 (t, J=8.0 Hz, 2H), 0.85 (t, J=8.0 Hz, 2H), 0.09 (s, 9H).

### Step 3: Synthesis of 2-[[7-fluoro-5-[4-[4-methyl-2-(pyridazin-3-yloxymethyl)thiazol-5-yl]thiazol-2-yl]benzotriazol-1-yl]methoxy]ethyl-trimethyl-silane (7c)

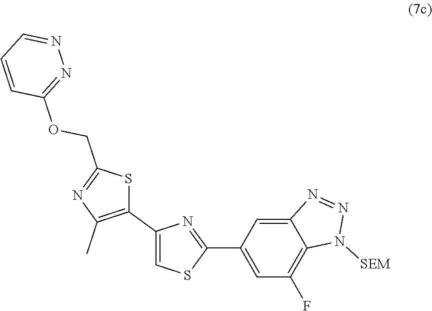 [SMILES: Cc1nc(COc2cccnn2)sc1-c1csc(-c2cc(F)c3c(c2)nnn3COCC[Si](C)(C)C)n1]

A mixture of bis(1-adamantyl)-butyl-phosphane (22.55 mg, 62.88 μmol, 0.2 eq) and diacetoxypalladium (14.12 mg, 62.88 μmol, 0.2 eq) in toluene (3 mL) was stirred at 20° C. for 0.5 h under N₂ atmosphere. The mixture was added to a mixture of 2-[[5-(4-bromothiazol-2-yl)-7-fluoro-benzotriazol-1-yl]methoxy]ethyl-trimethyl-silane 7b (150 mg, 314.41 μmol, 1 eq), 4,4,5,5-tetramethyl-2-(4,4,5,5-tetramethyl-1,3,2-dioxaborolan-2-yl)-1,3,2-dioxaborolane (319.36 mg, 1.26 mmol, 4 eq), CsF (143.27 mg, 943.22 μmol, 34.78 μL, 3 eq), 5-bromo-4-methyl-2-(pyridazin-3-yloxymethyl)thiazole 7a (149.94 mg, 471.61 μmol, 1.5 eq) in MeOH (6 mL) was stirred at 50° C. for 0.5 h, the mixture was stirred at 80° C. for 12 h. The reaction mixture was concentrated under reduced pressure to give a residue. The residue was purified with prep-HPLC (column: Boston Prime C18 150*30 mm*5 um; mobile phase: [water (0.05% ammonia hydroxide v/v)-ACN]; B %: 75%-95%, 10 min) to give 2-[[7-fluoro-5-[4-[4-methyl-2-(pyridazin-3-yloxymethyl)thiazol-5-yl]thiazol-2-yl]benzotriazol-1-yl]methoxy]ethyl-trimethyl-silane 7c (50 mg, 80.98 mol, 25.8% yield, 90% purity) as a solid. ¹H NMR (400 MHz, DMSO-d₆) δ ppm 9.09 (dd, J=1.2, 4.4 Hz, 1H), 8.68 (d, J=1.0 Hz, 1H), 8.28-8.12 (m, 2H), 7.83 (dd, J=4.4, 8.8 Hz, 1H), 7.51 (dd, J=1.2, 8.8 Hz, 1H), 6.22 (s, 2H), 5.92 (s, 2H), 3.74 (t, J=8.0 Hz, 2H), 2.78 (s, 3H), 0.96 (t, J=8.0 Hz, 2H), 0.00 (s, 9H).

Step 3: Synthesis of 5-[2-(7-fluoro-1H-benzotriazol-5-yl)thiazol-4-yl]-4-methyl-2-(pyridazin-3-yloxymethyl)thiazole (7). A mixture of 2-[[7-fluoro-5-[4-[4-methyl-2-(pyridazin-3-yloxymethyl)thiazol-5-yl]thiazol-2-yl]benzotriazol-1-yl]methoxy]ethyl-trimethyl-silane 7c (50 mg, 80.98 mol, 1 eq), TBAF (1 M in THF, 1.07 mL, 13.23 eq) in THF (2 mL) was degassed and purged with N₂ for 3 times, and then the mixture was stirred at 60° C. for 12 h. The mixture was concentrated and then water (10 mL) was added. The mixture was extracted with DCM (10 mL×3). The combined organic layers were washed with brine (10 mL), dried over Na₂SO₄, filtered and concentrated. The residue was purified with prep-HPLC (column: Boston Prime C18 150*30 mm*5 um; mobile phase: [water (0.05% ammonia hydroxide v/v)-ACN]; B %: 25%-45%, 10 min) to give 5-[2-(7-fluoro-1H-benzotriazol-5-yl)thiazol-4-yl]-4-methyl-2-(pyridazin-3-yloxymethyl)thiazole 7 (31.4 mg, 72.37 umol, 89.4% yield, 98.06% purity) as a solid. ¹H NMR (400 MHz, DMSO-d₆) δ ppm 8.99 (dd, J=1.4, 4.4 Hz, 1H), 8.29 (s, 1H), 8.04 (s, 1H), 7.80 (d, J=10.8 Hz, 1H), 7.72 (dd, J=4.8, 8.8 Hz, 1H), 7.41 (dd, J=1.4, 8.8 Hz, 1H), 5.81 (s, 2H), 2.67 (s, 3H); HPLC (98.06% purity); ES-MS m/z 448.0 [M+23]⁺.

### Example S8. Preparation of 5-[6-(4,7-difluoro-3H-benzotriazol-5-yl)-2-pyridyl]-4-methyl-2-[(5-methyl-pyrimidin-2-yl)oxymethyl]thiazole (8)

General Scheme for Compounds 8, 9, 12, 14, and 15.

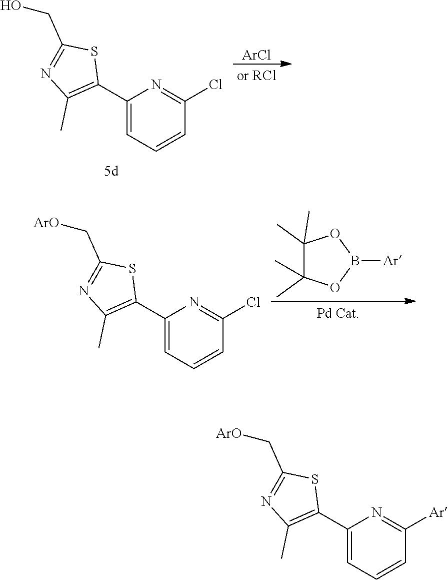

### Step 1: Synthesis of 5-(6-chloro-2-pyridyl)-4-methyl-2-[(5-methylpyrimidin-2-yl)oxymethyl]thiazole (8a)

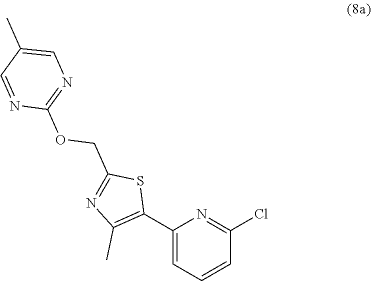 [SMILES: Cc1cnc(OCc2nc(C)c(-c3cccc(Cl)n3)s2)nc1]

To a solution of [5-(6-chloro-2-pyridyl)-4-methyl-thiazol-2-yl]methanol 5d (1 g, 4.15 mmol, 1 eq) in DMF (10 mL) was added NaH (332.32 mg, 8.31 mmol, 60% purity, 2 eq) at 0° C. and stirred for 0.5 h, and 2-chloro-5-methyl-pyrimidine (1.34 g, 10.39 mmol, 2.5 eq) was added. The mixture was stirred at 20° C. for 2 h. LCMS showed desired MS was detected. The reaction mixture was poured into ammonium chloride saturated aqueous solution (50 mL) and extracted with ethyl acetate (50 mL*3). The combined organic layer was washed with brine (50 mL), dried over anhydrous sodium sulfate, filtered and concentrated. The residue was purified by column chromatography (SiO₂, DCM/MeOH=0/1 to 10/1) to give 5-(6-chloro-2-pyridyl)-4-methyl-2-[(5-methylpyrimidin-2-yl)oxymethyl]thiazole 8a (0.8 g, 2.40 mmol, 57.86% yield) as a solid. ¹H NMR (400 MHz, CHLOROFORM-d) δ=8.31 (s, 2H), 7.60 (t, J=7.8 Hz, 1H), 7.36 (d, J=7.8 Hz, 1H), 7.13 (d, J=7.9 Hz, 1H), 5.60 (s, 2H), 2.63 (s, 3H), 2.19 (s, 3H).

Step 2: Synthesis of 5-[6-(4,7-difluoro-3H-benzotriazol-5-yl)-2-pyridyl]-4-methyl-2-[(5-methyl-pyrimidin-2-yl)oxymethyl]thiazole (8). A mixture of 5-(6-chloro-2-pyridyl)-4-methyl-2-[(5-methylpyrimidin-2-yl)oxymethyl]thiazole 8a (0.5 g, 1.50 mmol, 1 eq), 4,7-difluoro-6-(4,4,5,5-tetramethyl-1,3,2-dioxaborolan-2-yl)-1H-benzotriazole (2, Key Intermediate B, 760.08 mg, 2.70 mmol, 1.8 eq), Pd(dppf)Cl₂ (109.93 mg, 150.24 umol, 0.1 eq) and Cs₂CO₃ (1.22 g, 3.76 mmol, 2.5 eq) in dioxane (6 mL) and H₂O (2 mL) was degassed and purged with N₂ for 3 times, and then the mixture was stirred at 100° C. for 12 hr under N₂ atmosphere. LCMS showed desired MS was detected. The reaction mixture was concentrated under reduced pressure to give a residue. The residue was purified by flash silica gel chromatography (ISCO®; 40 g SepaFlash® Silica Flash Column, Eluent of 0˜15% DCM/MeOH ethergradient @ 70 mL/min). The cut fraction was concentrated under reduced pressure to give the crude product. The crude product was purified by triturated with MeOH (10 mL). Then filter cake was collected and concentrated under reduced pressure to give product. The crude product was purified by flash silica gel chromatography (ISCO®; 20 g SepaFlash® Silica Flash Column, Eluent of 0˜15% DCM/EA ether gradient @ 50 mL/min). The cut fraction was concentrated under reduced pressure to give 5-[6-(4,7-difluoro-3H-benzotriazol-5-yl)-2-pyridyl]-4-methyl-2-[(5-methylpyrimidin-2-yl)oxymethyl]thiazole 8 (250 mg, 553.77 umol, 36.86% yield, 98.20% purity) as a solid. ¹H NMR (400 MHz, DMSO-d6) δ=8.51 (s, 2H), 8.12-8.04 (m, 1H), 7.92 (dd, J=1.5, 7.8 Hz, 1H), 7.86-7.75 (m, 2H), 5.64 (s, 2H), 2.71 (s, 3H), 2.21 (s, 3H); HPLC (98.20% purity); ES-MS m/z 452.2 [M+H]⁺.

### Example S9. Preparation of 1-[[5-[6-(4,7-difluoro-3H-benzotriazol-5-yl)-2-pyridyl]-4-methyl-thiazol-2-yl]methoxy]-2-methyl-propan-2-ol (9)

### Step 1: Synthesis of ethyl 2-[[5-(6-chloro-2-pyridyl)-4-methyl-thiazol-2-yl]methoxy]acetate (9a)

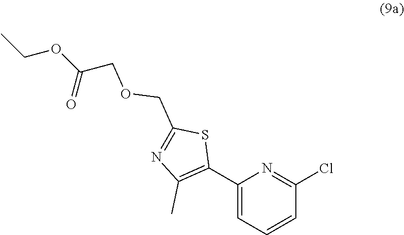 [SMILES: CCOC(=O)COCc1nc(C)c(-c2cccc(Cl)n2)s1]

A mixture of [5-(6-chloro-2-pyridyl)-4-methyl-thiazol-2-yl]methanol (from Example S5, intermediate 5d, 1 g, 4.15 mmol, 1 eq) in DMF (6 mL) cooled to 0° C. was added NaH (332.35 mg, 8.31 mmol, 60% purity, 2 eq) in portions under N₂ atmosphere. The mixture was stirred at 0° C. for 15 min under N₂ atmosphere. Then a solution ethyl 2-bromoacetate (901.92 mg, 5.40 mmol, 597.30 uL, 1.3 eq) was added dropwise to the mixture. The whole mixture was stirred at 0° C. for 1 h under N₂ atmosphere. Then the whole mixture was stirred at 25° C. for 1 h under N₂ atmosphere. LCMS showed material was consumed completely and one main peak with desired MS was detected. TLC (PE:EA=1:1) indicated material was consumed completely and one new spot formed. The reaction mixture was quenched by addition NH₄Cl solution (15 mL) at 0° C., and then extracted with ethyl acetate (10 mL×3). The combined organic layers were washed with brine (30 mL), dried over Na₂SO₄, filtered and concentrated under reduced pressure to give a residue and purified by flash silica gel chromatography (Eluent of 0˜35% Ethyl acetate/Petroleum ether gradient) to give ethyl 2-[[5-(6-chloro-2-pyridyl)-4-methyl-thiazol-2-yl]methoxy]acetate 9a (600 mg, 1.84 mmol, 44.19% yield) as an oil. ¹H NMR (400 MHz, CHLOROFORM-d) δ=7.70 (t, J=7.8 Hz, 1H), 7.45 (d, J=7.5 Hz, 1H), 7.24 (d, J=7.5 Hz, 1H), 4.92 (s, 2H), 4.30-4.20 (m, 4H), 2.71 (s, 3H), 1.31 (t, J=7.2 Hz, 3H).

### Step 2: Synthesis of 1-[[5-(6-chloro-2-pyridyl)-4-methyl-thiazol-2-yl]methoxy]-2-methyl-propan-2-ol (9b)

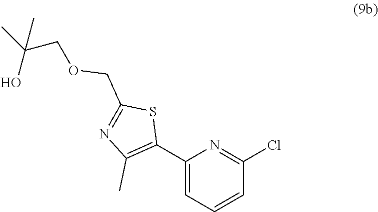 [SMILES: Cc1nc(COCC(C)(C)O)sc1-c1cccc(Cl)n1]

To a solution of ethyl 2-[[5-(6-chloro-2-pyridyl)-4-methyl-thiazol-2-yl]methoxy]acetate 9a (600 mg, 1.84 mmol, 1 eq) in THF (10 mL) at 0° C. was added MeMgBr (3 M, 1.84 mL, 3 eq) dropwise. The mixture was stirred at 25° C. for 3 h. TLC (PE:EA=0:1) indicated material was consumed completely and one new main spot formed. LCMS showed material was consumed completely and one main peak with desired MS was detected. The reaction mixture was quenched by addition NH₄Cl saturated solution (5 mL) at 0° C., and then diluted with water (15 mL) and extracted with ethyl acetate (20 mL×3). The combined organic layers were washed with brine (50 mL), dried over Na₂SO₄, filtered and concentrated under reduced pressure to give a residue. Then purified by flash silica gel chromatography (Eluent of 10-40% Ethyl acetate/Petroleum ether gradient) to give 1-[[5-(6-chloro-2-pyridyl)-4-methyl-thiazol-2-yl]methoxy]-2-methyl-propan-2-ol 9b (350 mg, 1.12 mmol, 60.94% yield) as an oil. ¹H NMR (400 MHz, CHLOROFORM-d) δ=7.72 (t, J=7.8 Hz, 1H), 7.47 (d, J=7.6 Hz, 1H), 7.25 (s, 1H), 4.91 (s, 2H), 3.50 (s, 2H), 2.79-2.67 (m, 3H), 1.27 (s, 6H).

Step 3: Synthesis of 1-[[5-[6-(4,7-difluoro-3H-benzotriazol-5-yl)-2-pyridyl]-4-methyl-thiazol-2-yl]methoxy]-2-methyl-propan-2-ol (9). A mixture of 1-[[5-(6-chloro-2-pyridyl)-4-methyl-thiazol-2-yl]methoxy]-2-methyl-propan-2-ol 9b (87 mg, 278.12 umol, 1 eq), 4,7-difluoro-6-(4,4,5,5-tetramethyl-1,3,2-dioxaborolan-2-yl)-1H-benzotriazole (2, Key Intermediate B, 156.34 mg, 556.24 umol, 2 eq), Cs₂CO₃ (271.85 mg, 834.36 umol, 3 eq) and Pd(dppf)Cl₂·CH₂Cl₂ (113.56 mg, 139.06 umol, 0.5 eq) in dioxane (1 mL)/H₂O (0.2 mL) was degassed and purged with N₂ for 3 times, and then the mixture was stirred at 105° C. for 12 h under N₂ atmosphere. LCMS showed material was consumed completely and desired compound was detected. The reaction mixture was filtered and concentrated under reduced pressure to give a residue and purified by prep-HPLC (basic condition: column: Waters Xbridge C18 150*50 mm*10 um; mobile phase: [water (0.05% ammonia hydroxide v/v)-ACN]; B %: 5%-35%, 11 min). Then re-purified by prep-TLC (SiO₂, Ethyl acetate: MeOH=4:1) to give 1-[[5-[6-(4,7-difluoro-3H-benzotriazol-5-yl)-2-pyridyl]-4-methyl-thiazol-2-yl]methoxy]-2-methyl-propan-2-ol 9 (35 mg, 77.79 umol, 6.99% yield, 95.9% purity) as a solid. ¹H NMR (400 MHz, DMSO-d6) δ=8.10-8.03 (m, 1H), 7.91 (dd, J=1.4, 7.9 Hz, 1H), 7.81-7.74 (m, 2H), 4.80 (s, 2H), 4.47 (s, 1H), 3.36 (br s, 2H), 2.69 (s, 3H), 1.13 (s, 6H); HPLC (95.91% purity); ES-MS m/z 432.4 [M+H]⁺.

### Example S10. Preparation of 2,3,6-trifluoro-4-[4-[4-methyl-2-(pyrimidin-2-yloxymethyl)thiazol-5-yl]thiazol-2-yl]phenol (10)

### Step 1: Synthesis of 5-bromo-4-methyl-2-(pyrimidin-2-yloxymethyl)thiazole (10a)

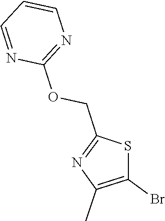

To a mixture of (5-bromo-4-methyl-thiazol-2-yl)methanol 3e (500 mg, 2.40 mmol, 1 eq) in THF (12.5 mL) was added NaH (144.00 mg, 3.60 mmol, 60% purity, 1.5 eq) at 0° C. and purged with N₂ for 3 times, and then the mixture was stirred at 15° C. for 0.5 h. 2-chloropyrimidine (329.86 mg, 2.88 mmol, 1.2 eq) was added to the reaction mixture, the mixture was stirred at 15° C. for 12 h. TLC (PE/EtOAc=2:1) showed a new spot was formed, and the desired MS was observed on LCMS. The reaction mixture was poured into ice-water (30 mL), extracted with EtOAc (50 mL×3). The combined organic layers were washed with brine (50 mL), dried over Na₂SO₄, filtered and concentrated. The residue was purified by clumn chromatography (SiO₂, PE/EtOAc=4:1) to give 5-bromo-4-methyl-2-(pyrimidin-2-yloxymethyl)thiazole 10a (534 mg, 1.87 mmol, 77.76% yield) as a solid. ¹H NMR (400 MHz, CHLOROFORM-d) δ=8.58 (d, J=4.6 Hz, 2H), 7.03 (t, J=4.8 Hz, 1H), 5.63 (s, 2H), 2.41 (s, 3H).

Step 2: Synthesis of 2,3,6-trifluoro-4-[4-[4-methyl-2-(pyrimidin-2-yloxymethyl)thiazol-5-yl]thiazol-2-yl]phenol (10). A mixture of bis(1-adamantyl)-butyl-phosphane (62.44 mg, 174.14 mol, 0.2 eq) and Pd(OAc)₂ (19.55 mg, 87.07 μmol, 0.1 eq) in anhydrous toluene (30 mL) was stirred at 20° C. for 0.5 h under N₂ atmosphere. The above solution was added to a mixture of 4-(4-bromothiazol-2-yl)-2,3,6-trifluoro-phenol (4, Key Intermediate D, 300 mg, 870.71 μmol, 1 eq), 4,4,5,5-tetramethyl-2-(4,4,5,5-tetramethyl-1,3,2-dioxaborolan-2-yl)-1,3,2-dioxaborolane (884.43 mg, 3.48 mmol, 4 eq), CsF (396.78 mg, 2.61 mmol, 96.31 μL, 3 eq), 5-bromo-4-methyl-2-(pyrimidin-2-yloxymethyl)thiazole 10a (332.20 mg, 1.04 mmol, 1.2 eq) in anhydrous MeOH (100 mL) and then the mixture was stirred at 80° C. for 12 h under N₂ atmosphere. The reaction mixture was concentrated and then water (200 mL) was added. The mixture was extracted with DCM (200 mL×3). The combined organic layers were washed with brine (200 mL), dried over Na₂SO₄, filtered and concentrated. The residue was purified with prep-HPLC (column: Boston Prime C18 150*30 mm*5 um;mobile phase: [water(0.2% FA)-ACN]; B %: 46%-69%, 10 min) to give 2,3,6-trifluoro-4-[4-[4-methyl-2-(pyrimidin-2-yloxymethyl)thiazol-5-yl]thiazol-2-yl]phenol 10 (49 mg, 110.03 μmol, 12.6% yield, 98% purity) of this batch as a solid. ¹H NMR (400 MHz, DMSO-d₆) δ ppm 8.70 (d, J=4.8 Hz, 2H), 8.03 (s, 1H), 7.75-7.71 (m, 1H), 7.25 (t, J=4.8 Hz, 1H), 5.66 (s, 2H), 2.63 (s, 3H); HPLC (100.00% purity); ES-MS m/z 437.1 [M+H]⁺.

### Example 511. Preparation of 2,3,6-trifluoro-4-[4-[4-methyl-2-(2-pyridyloxymethyl)thiazol-5-yl]thiazol-2-yl]phenol (11)

### Step 1: Synthesis of 5-bromo-4-methyl-2-(2-pyridyloxymethyl)thiazole (11a)

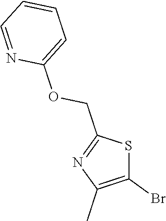

To a solution of (5-bromo-4-methyl-thiazol-2-yl)methanol 3e (0.4 g, 1.73 mmol, 1 eq) and pyridin-2-ol (329.07 mg, 3.46 mmol, 2 eq) in THF (4 mL) were added DEAD (602.64 mg, 3.46 mmol, 629.06 mL, 2 eq) and PPh₃ (907.60 mg, 3.46 mmol, 2 eq) under N₂. The reaction mixture was stirred at 20° C. for 1 h under N₂. The reaction mixture was concentrated under reduced pressure. The residue was purified with flash silica gel chromatography (ISCO®; 20 g SepaFlash® Silica Flash Column, Eluent of 0˜35% Ethylacetate/Petroleum ether gradient @ 35 mL/min) to give 5-bromo-4-methyl-2-(2-pyridyloxymethyl)thiazole 11a (130 mg, 410.30 mmol, 23.7% yield, 90% purity) as a solid. ¹HNMR (400 MHz, CDCl₃) δ ppm 8.22-8.14 (m, 1H), 7.65-7.57 (m, 1H), 6.96-6.91 (m, 1H), 6.83 (d, J=8.4 Hz, 1H), 5.57 (s, 2H), 2.40 (s, 3H).

Step 2: Synthesis of 2,3,6-trifluoro-4-[4-[4-methyl-2-(2-pyridyloxymethyl)thiazol-5-yl]thiazol-2-yl]phenol (11). A mixture of bis(1-adamantyl)-butyl-phosphane (31.22 mg, 87.07 mol, 0.2 eq) and diacetoxypalladium (19.55 mg, 87.07 umol, 0.2 eq) in toluene (6 mL) was stirred at 20° C. for 0.5 h under N₂ atmosphere, the mixture was added to a mixture of 5-bromo-4-methyl-2-(2-pyridyloxymethyl)thiazole 11a (169.88 mg, 565.96 mol, 1.3 eq), 4,4,5,5-tetramethyl-2-(4,4,5,5-tetramethyl-1,3,2-dioxaborolan-2-yl)-1,3,2-dioxaborolane (442.21 mg, 1.74 mmol, 4 eq), CsF (198.39 mg, 1.31 mmol, 48.15 μL, 3 eq), 4-(4-bromothiazol-2-yl)-2,3,6-trifluoro-phenol (4, Key Intermediate D, 150 mg, 435.36 mol, 1 eq) in MeOH (4 mL), the mixture was stirred at 80° C. for 1 h under microwave (2 bar). The mixture was concentrated and then water (50 mL) was added. The mixture was extracted with DCM (50 mL×3). The combined organic layers were washed with brine (50 mL), dried over Na₂SO₄, filtered and concentrated. The residue was purified with prep-HPLC (column: YMC-Actus Triart C18 150*30 mm*7 um; mobile phase: [water (0.05% ammonia hydroxide v/v)-ACN]; B %: 14%-54%, 11 min) to give the crude product. The crude product was purified with prep-HPLC (column: Phenomenex Gemini-NX 150*30 mm*5 um;mobile phase: [water (0.05% ammonia hydroxide v/v)-ACN]; B %: 12%-52%, 10 min) to give 2,3,6-trifluoro-4-[4-[4-methyl-2-(2-pyridyloxymethyl)thiazol-5-yl]thiazol-2-yl]phenol 11 (27.6 mg, 63.01 umol, 14.5% yield, 99.41% purity) as a solid. ¹H NMR (400 MHz, DMSO-d₆) δ ppm 11.67 (br s, 1H), 8.23 (dd, J=1.6, 4.8 Hz, 1H), 8.01 (s, 1H), 7.83-7.67 (m, 2H), 7.07 (dd, J=5.6, 6.8 Hz, 1H), 6.98 (d, J=8.4 Hz, 1H), 5.62 (s, 2H), 2.63 (s, 3H); HPLC (Purity: 99.41%); ES-MS m/z 341.0 [M+H]⁺.

### Example S12. Preparation of 3-fluoro-2-hydroxy-5-[6-[4-methyl-2-(pyrimidin-2-yloxymethyl)thiazol-5-yl]-2-pyridyl]benzonitrile (12)

### Step 1: Synthesis of 3-fluoro-2-hydroxy-benzonitrile (12a)

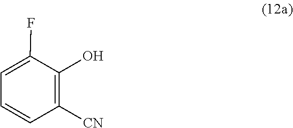

To a solution of 2,3-difluorobenzonitrile (500 mg, 3.59 mmol, 400.00 μL, 1 eq) in DMF (15 mL) was added 2-methylsulfonylethanol (446.29 mg, 3.59 mmol, 1 eq) and NaH (287.53 mg, 7.19 mmol, 60% purity, 2 eq) in small portions at 25° C. The reaction mixture was stirred at 25° C. for 0.5 h. The reaction mixture was poured into water (30 mL) and washed with ethyl acetate (30 mL×3), the aqueous phase was adjusted with pH=2 with 1 N HCl and extracted with ethyl acetate (30 mL×3). The combined organic layers were dried over Na₂SO₄, filtered, filtrate was concentrated under reduced pressure to give 3-fluoro-2-hydroxy-benzonitrile 12a (350 mg, 2.30 mmol, 63.9% yield, 90% purity) as a solid. ¹H NMR (400 MHz, DMSO-d₆) δ ppm 11.45 (br s, 1H), 7.54-7.44 (m, 1H), 7.47-7.42 (m, 1H), 6.94 (dt, J=4.8, 8.0 Hz, 1H).

### Step 2: Synthesis of 5-bromo-3-fluoro-2-hydroxy-benzonitrile (12b)

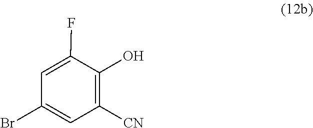 [SMILES: N#Cc1cc(Br)cc(F)c1O]

To a solution of 3-fluoro-2-hydroxy-benzonitrile 12a (350 mg, 2.30 mmol, 1 eq) in CH₃CN (5 mL) was added NBS (449.78 mg, 2.53 mmol, 1.1 eq) in one portion at 20° C. The reaction mixture was stirred at 20° C. for 1 h. The reaction mixture was poured into sat. Na₂CO₃ (20 mL) and washed with ethyl acetate (20 mL×3). Then the aqueous phase was adjusted to pH=2 with 1 N HCl and extracted with ethyl acetate (20 mL×3), the combined organic layers were dried over Na₂SO₄, filtered, filtrate was concentrated to give 5-bromo-3-fluoro-2-hydroxy-benzonitrile 12b (300 mg, 1.19 mmol, 52.0% yield, 86% purity) as a solid. ¹H NMR (400 MHz, DMSO-d₆) δ ppm 11.87 (br. s, 1H), 7.88-7.85 (m, 1H), 7.77 (d, J=1.6 Hz, 1H).

### Step 3: Synthesis of 3-fluoro-2-hydroxy-5-(4,4,5,5-tetramethyl-1,3,2-dioxaborolan-2-yl)benzonitrile (12c)

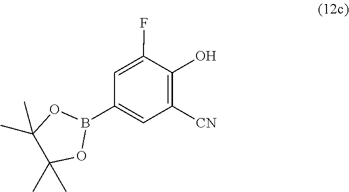 [SMILES: CC1(C)OB(c2cc(F)c(O)c(C#N)c2)OC1(C)C]

A mixture of 5-bromo-3-fluoro-2-hydroxy-benzonitrile 12b (250 mg, 995.34 μmol, 1 eq), 4,4,5,5-tetramethyl-2-(4,4,5,5-tetramethyl-1,3,2-dioxaborolan-2-yl)-1,3,2-dioxaborolane (631.88 mg, 2.49 mmol, 2.5 eq), KOAc (293.05 mg, 2.99 mmol, 3 eq) and Pd(dppf)Cl₂·CH₂Cl₂ (81.28 mg, 99.53 μmol, 0.1 eq) in 1,4-dioxane (10 mL) was degassed and purged with N₂ for 3 times, and then the mixture was stirred at 85° C. for 12 h under N₂ atmosphere. TLC (petroleum ether/ethyl acetate=1/1) showed the reaction was completed. The reaction mixture was concentrated under reduced pressure to give 3-fluoro-2-hydroxy-5-(4,4,5,5-tetramethyl-1,3,2-dioxaborolan-2-yl)benzonitrile 12c (300 mg, crude) as a gum. ¹H NMR (400 MHz, CDCl₃) δ ppm 7.75 (s, 1H), 7.64 (d, J=10.4 Hz, 1H), 7.43-7.38 (m, 1H), 1.33 (s, 12H).

Step 4: Synthesis of 3-fluoro-2-hydroxy-5-[6-[4-methyl-2-(pyrimidin-2-yloxymethyl)thiazol-5-yl]-2-pyridyl]benzonitrile (12). A mixture of 5-(6-chloro-2-pyridyl)-4-methyl-2-(pyrimidin-2-yloxymethyl)thiazole (5, Key Intermediate E, 250 mg, 705.81 μmol, 1 eq), 3-fluoro-2-hydroxy-5-(4,4,5,5-tetramethyl-1,3,2-dioxaborolan-2-yl)benzonitrile 12c (278.52 mg, 1.06 mmol, 1.5 eq), Cs₂CO₃ (459.94 mg, 1.41 mmol, 2 eq) and Pd(dppf)Cl₂·CH₂Cl₂ (57.64 mg, 70.58 μmol, 0.1 eq) in 1,4-dioxane (9 mL) and H₂O (3 mL) was degassed and purged with N₂ for 3 times, and then the mixture was stirred at 85° C. for 12 h under N₂ atmosphere. The reaction mixture was concentrated under reduced pressure. The residue was purified with reversed-phase HPLC (H₂O-ACN condition) to give crude product, which was purified with prep-HPLC (column: Boston Prime C18 150*30 mm*5 m; mobile phase: [water (0.05% NH₃H₂O+10 mM NH₄HCO₃)-ACN]; B %: 27%-50%, 7 min) to give 3-fluoro-2-hydroxy-5-[6-[4-methyl-2-(pyrimidin-2-yloxymethyl)thiazol-5-yl]-2-pyridyl]benzonitrile 12 (71.8 mg, 166.89 μmol, 23.6% yield, 97.49% purity) as a solid. ¹H NMR (400 MHz, DMSO-d₆) δ ppm 8.69 (d, J=4.8 Hz, 2H), 7.79 (d, J=2.4 Hz, 1H), 7.78-7.75 (m, 1H), 7.76 (d, J=7.6 Hz, 1H), 7.75-7.70 (m, 1H), 7.67 (d, J=8.0 Hz, 1H), 7.40 (d, J=7.6 Hz, 1H), 7.23 (t, J=4.8 Hz, 2H), 5.64 (s, 2H), 2.66 (s, 3H); HPLC (Purity: 97.49%); ES-MS m/z 420.2 [M+H]⁺.

### Example S13. Preparation of methyl((4′-methyl-2-(4-((phenylsulfonyl)carbamoyl)phenyl)-[4,5′-bithiazol]-2′-yl)methyl)carbamate (13)

General Scheme for Compounds 13 and 16.

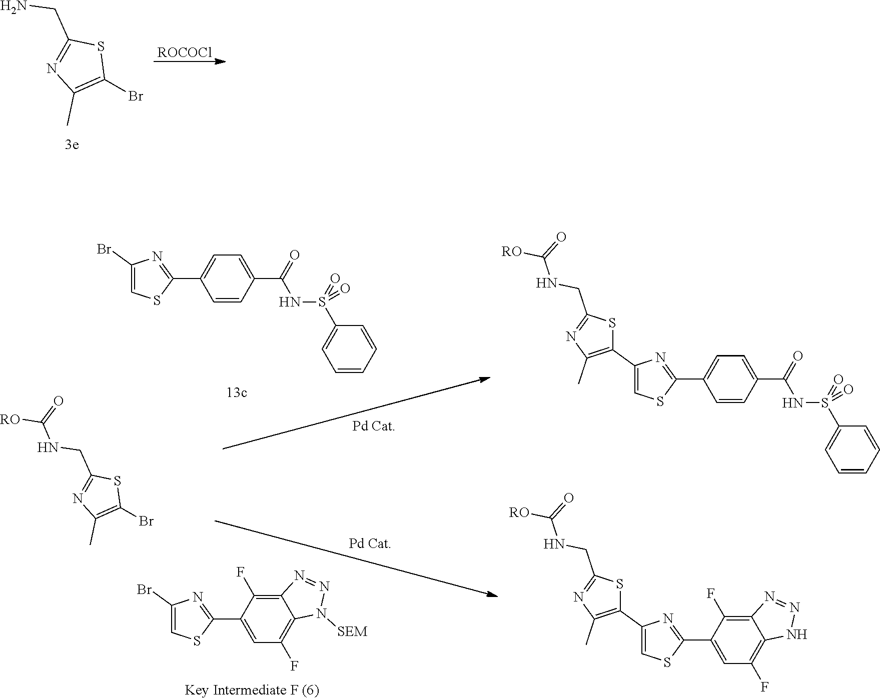

### Step 1: Synthesis of 4-bromo-N-(phenylsulfonyl)benzamide (13a)

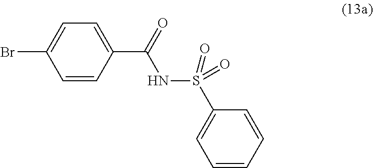 [SMILES: O=C(NS(=O)(=O)c1ccccc1)c1ccc(Br)cc1]

To a solution of 4-bromobenzoyl chloride (3 g, 13.67 mmol, 1 eq) and benzenesulfonamide (2.36 g, 15.04 mmol, 1.1 eq) in DCM (30 mL) was added DIEA (5.30 g, 41.01 mmol, 7.14 mL, 3 eq). The mixture was stirred at 25° C. for 2 h. TLC (PE/EtOAc=1/1) indicated starting material was consumed completely, and one major new spot with larger polarity was formed. The mixture was added H₂O (10 mL) and extracted with EtOAc (30 mL×3). The combined organic layers were washed with H₂O (100 mL×3) and brine (100 mL×2), dried over Na₂SO₄, filtered and concentrated. The residue was purified by flash silica gel chromatography (ISCO®; 40 g SepaFlash® Silica Flash Column, Eluent of 0-50% Ethyl acetate/Petroleum ether gradient @ 40 mL/min) to give N-(benzenesulfonyl)-4-bromo-benzamide 13a (4.5 g, 11.91 μmol, 87.1% yield, 90% purity) as a solid. ¹H NMR (400 MHz, DMSO-d₆) δ ppm 7.86-7.80 (dd, J=8, 12 Hz, 4H), 7.53 (d, J=8.4 Hz, 2H), 7.40-7.47 (d, J=5.6 Hz, 3H).

### Step 2: Synthesis of N-(phenylsulfonyl)-4-(4,4,5,5-tetramethyl-1,3,2-dioxaborolan-2-yl)benzamide (13b)

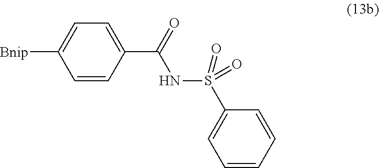

A mixture of N-(benzenesulfonyl)-4-bromo-benzamide 13a (5.2 g, 13.76 mmol, 1 eq), 4,4,4′,4′,5,5,5′,5′-octamethyl-2,2′-bi(1,3,2-dioxaborolane) (6.99 g, 27.51 mmol, 2 eq), Pd(dppf)Cl₂ (1.01 g, 1.38 mmol, 0.1 eq), KOAc (2.70 g, 27.51 mmol, 2 eq) in dioxane (100 mL) was degassed and purged with N₂ for 3 times, and then the mixture was stirred at 80° C. for 12 h under N₂ atmosphere. TLC (PE/EtOAc=1/1) indicated starting material was consumed completely, and one major new spot with lower polarity was formed. The mixture was concentrated and then water (100 mL) was added. The mixture was extracted with EtOAc (100 mL×3). The combined organic layers were washed with brine (200 mL), dried over Na₂SO₄, filtered and concentrated. The residue was purified by flash silica gel chromatography (ISCO®; 40 g SepaFlash® Silica Flash Column, Eluent of 0˜50% Ethyl acetate/Petroleum ether gradient @ 40 mL/min) to give N-(benzenesulfonyl)-4-(4,4,5,5-tetramethyl-1,3,2-dioxaborolan-2-yl)benzamide 13b (5.9 g, 13.71 mmol, 99.7% yield, 90% purity) as a solid. ¹H NMR (400 MHz, CDCl₃) δ ppm 8.17 (d, J=8 Hz, 2H), 7.89-7.83 (m, 2H), 7.80-7.74 (m, 2H), 7.66 (s, 1H), 7.59-7.53 (m, 2H), 1.34 (s, 12H).

### Step 3: Synthesis of 4-(4-bromothiazol-2-yl)-N-(phenylsulfonyl)benzamide (13c)

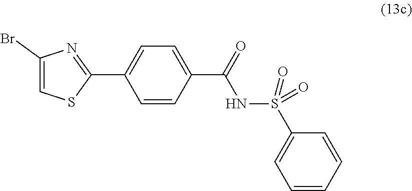 [SMILES: O=C(NS(=O)(=O)c1ccccc1)c1ccc(-c2nc(Br)cs2)cc1]

A mixture of N-(benzenesulfonyl)-4-bromo-benzamide 13b (500 mg, 1.32 μmol, 1 eq), 4,4,4′,4′,5,5,5′,5′-octamethyl-2,2′-bi(1,3,2-dioxaborolane) (671.81 mg, 2.65 μmol, 2 eq), Pd(dppf)Cl₂ (96.79 mg, 132.28 μmol, 0.1 eq), KOAc (259.64 mg, 2.65 μmol, 2 eq) in dioxane (10 mL) was degassed and purged with N₂ for 3 times, and then the mixture was stirred at 80° C. for 12 h under N₂ atmosphere. TLC (PE/EtOAc=1/1) indicated starting material was remained, and one major new spot with lower polarity was formed. The mixture was concentrated and then water (10 mL) was added. The mixture was extracted with EtOAc (10 mL×3). The combined organic layers were washed with brine (20 mL), dried over Na₂SO₄, filtered and concentrated. The residue was purified by flash silica gel chromatography (ISCO®; 12 g SepaFlash® Silica Flash Column, Eluent of 0-50% Ethyl acetate/Petroleum ether gradient @ 25 mL/min) to give N-(benzenesulfonyl)-4-(4,4,5,5-tetramethyl-1,3,2-dioxaborolan-2-yl)benzamide 13c (510 mg, 1.19 μmol, 89.6% yield, 90% purity) as a solid. ¹H NMR (400 MHz, DMSO-d₆) δ ppm=8.05-7.92 (m, 5H), 7.88-7.47 (m, 5H).

Step 4: Synthesis of methyl((4′-methyl-2-(4-((phenylsulfonyl)carbamoyl)phenyl)-[4,5′-bithiazol]-2′-yl)methyl)carbamate (13). A solution of bis(1-adamantyl)-butylphosphane (45.74 mg, 127.57 μmol, 0.2 eq) and diacetoxypalladium (28.64 mg, 127.57 μmol, 0.2 eq) in toluene (17.5 mL) was stirred at 20° C. for 0.5 h under N₂. The mixture was added to a solution of N-(benzenesulfonyl)-4-(4-bromothiazol-2-yl)benzamide 13c (300 mg, 637.84 μmol, 1 eq), 4,4,5,5-tetramethyl-2-(4,4,5,5-tetramethyl-1,3,2-dioxaborolan-2-yl)-1,3,2-dioxaborolane (647.89 mg, 2.55 mmol, 4 eq), methyl N-[(5-bromo-4-methyl-thiazol-2-yl)methyl]carbamate 16a (263.06 mg, 892.98 μmol, 1.4 eq) and CsF (290.67 mg, 1.91 mmol, 70.55 μL, 3 eq) in MeOH (30 mL) at 50° C. under N₂. The mixture was stirred at 80° C. for 12 h. The mixture was concentrated and then DMSO (4 mL) was added. The mixture was filtered and the filtrate was purified by prep-HPLC (column: YMC-Triart Prep C18 150*40 mm*7 um;mobile phase: [water(0.225% FA)-ACN];B %: 45%-75%, 8 min) to give methyl N-[[5-[2-[4-(benzenesulfonylcarbamoyl)phenyl]thiazol-4-yl]-4-methyl-thiazol-2-yl]methyl]carbamate 13 (31.7 mg, 59.04 μmol, 9.3% yield, 98.45% purity) as a solid. ¹H NMR (400 MHz, DMSO-d₆) δ ppm 8.07 (d, J=6.8 Hz, 3H), 8.05-7.97 (m, 5H), 7.76-7.69 (m, 1H), 7.69-7.61 (m, 2H), 4.42 (d, J=4.0 Hz, 2H), 3.60 (s, 3H), 2.60 (s, 3H); HPLC (98.45% purity); ES-MS m/z 529.1 [M+H]⁺.

Example S14. Preparation of 5-(6-(1H-benzo[d][1,2,3]triazol-6-yl)pyridin-2-yl)-4-methyl-2-((pyrimidin-2-yloxy)methyl)thiazole (14). To a 2.0 mL microwave tube was added 5-(6-chloropyridin-2-yl)-4-methyl-2-((pyrimidin-2-yloxy)methyl)thiazole (5, Key Intermediate E, 80 mg, 250.96 μmol), 6-(4,4,5,5-tetramethyl-1,3,2-dioxaborolan-2-yl)-1H-benzo[d][1,2,3]triazole (67.66 mg, 276.05 μmol), potassium carbonate (34.68 mg, 250.96 μmol), water (0.5 ml) and dioxane (1.5 ml). PdCl₂(dppf) (18.36 mg, 25.10 μmol) was finally added and the vial sealed and heated to 80° C. with stirring. After a total of 26 hr., the mixture was allowed to cool to room temp and then diluted with water and extracted with EtAc. The extracts were then washed once with water followed by brine solution, dried (Na2SO4), filtered and concentrated in vacuo. The crude product was chromatographed (ISCO, dry loading, 4 g silica column, gradient=60% EtAc/heptane to 100% EtAc, 18 min run time). The fractions containing product were then concentrated in vacuo to give 5-(6-(1H-benzo[d][1,2,3]triazol-6-yl)pyridin-2-yl)-4-methyl-2-((pyrimidin-2-yloxy)methyl)thiazole 14 (10 mg, 24.91 μmol, 9.9% yield) as a solid. ¹H NMR (400 MHz, DMSO-d6) δ 15.88 (s, 1H), 8.70 (d, J=4.7 Hz, 3H), 8.41-7.94 (m, 4H), 7.73 (d, J=7.7 Hz, 1H), 7.24 (t, J=4.8 Hz, 1H), 5.68 (s, 2H), 2.73 (s, 3H). ES-MS m/z 402.4 [M+H]⁺.

### Example S15. Preparation of 3-(4-(6-(4-methyl-2-((pyrimidin-2-yloxy)methyl)thiazol-5-yl)pyridin-2-yl)phenyl)-1,2,4-oxadiazol-5(4H)-one (15)

### Step 1: Synthesis of 3-(4-(4,4,5,5-tetramethyl-1,3,2-dioxaborolan-2-yl)phenyl)-1,2,4-oxadiazol-5(4H)-one (15a)

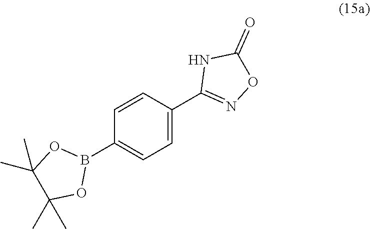 [SMILES: CC1(C)OB(c2ccc(-c3noc(=O)[nH]3)cc2)OC1(C)C]

In a 5 mL microwave vial were placed bis(pinacolato)diboron (702.33 mg, 2.74 mmol), 3-(4-bromophenyl)-1,2,4-oxadiazol-5(4H)-one (330 mg, 1.37 mmol), potassium acetate (537.44 mg, 5.48 mmol) and DMSO (5 mL). After the mixture was purged with nitrogen for 5 min, PdCl₂(dppf) (75.13 mg, 102.68 μmol) was added at 20° C. After 20 h at 80° C., the mixture was poured into water (30 mL), stirred for 30 min, filtered, and dried to give 3-(4-(4,4,5,5-tetramethyl-1,3,2-dioxaborolan-2-yl)phenyl)-1,2,4-oxadiazol-5(4H)-one 15a (540 mg, 1.87 mmol, 136.9% yield), which was used in the next step without further purification. ES-MS m/z 289.2 [M+H]⁺.

Step 2: Synthesis of 3-(4-(6-(4-methyl-2-((pyrimidin-2-yloxy)methyl)thiazol-5-yl)pyridin-2-yl)phenyl)-1,2,4-oxadiazol-5(4H)-one (15). In a 5 mL microwave vial were placed 5-(6-chloropyridin-2-yl)-4-methyl-2-((pyrimidin-2-yloxy)methyl)thiazole (5, Key Intermediate E, 30 mg, 94.11 μmol), 3-(4-(4,4,5,5-tetramethyl-1,3,2-dioxaborolan-2-yl)phenyl)-1,2,4-oxadiazol-5(4H)-one 15a (54.23 mg, 188.22 μmol), cesium carbonate (76.66 mg, 235.27 μmol), water (0.6 ml), and 1,4-dioxane (3 ml). After the mixture was bubbled with nitrogen gas for 5 min, PalladiumTetrakis (10.88 mg, 9.41 μmol) was added at 20° C. After 15 h at 90° C., the mixture was diluted with EtOAc (20 mL) and water (10 mL), and acidified with 10% citric acid. The organic layer was separated, dried (Na₂SO₄), filtered, and concentrated in a rotavap to give a residue, which was purified by HPLC to afford 3-(4-(6-(4-methyl-2-((pyrimidin-2-yloxy)methyl)thiazol-5-yl)pyridin-2-yl)phenyl)-1,2,4-oxadiazol-5(4H)-one 15 (9.2 mg, 20.70 μmol, 22.0% yield) as a solid after lyophilization. ¹H NMR (400 MHz, DMSO-d6) δ 8.72 (d, J=4.8 Hz, 2H), 8.32 (d, J=8.2 Hz, 2H), 8.06 (d, J=5.8 Hz, 2H), 7.98 (d, J=8.2 Hz, 2H), 7.77 (dd, J=5.8, 2.8 Hz, 1H), 7.26 (t, J=4.8 Hz, 1H), 5.70 (s, 2H), 2.74 (s, 3H), 2.56 (s, 1H). ES-MS m/z 445.3 [M+H]⁺.

### Example S16. Preparation of methyl N-[[5-[2-(4,7-difluoro-1H-benzotriazol-5-yl)thiazol-4-yl]-4-methyl-thiazol-2-yl]methyl]carbamate (16)

### Step 1: Synthesis of methyl N-[(5-bromo-4-methyl-thiazol-2-yl)methyl]carbamate (16a)

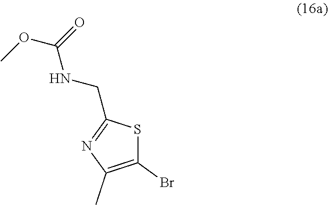

To a solution of (5-bromo-4-methyl-thiazol-2-yl)methanamine (3, Key Intermediate C, 1 g, 4.83 mmol, 1 eq) in DCM (20 mL) was added TEA (2.44 g, 24.14 mmol, 3.36 mL, 5 eq) and methyl chloroformate (1.37 g, 14.49 mmol, 1.12 mL, 3 eq) at 0° C. The mixture was stirred at 25° C. for 2 hr. LCMS showed the desired mass was detected. The reaction mixture was filtered and concentrated under reduced pressure to give a residue. The residue was poured into ammonium chloride saturated aqueous solution (50 mL) and extracted with ethyl acetate (50 mL×3). The combined organic layer was washed with brine (50 mL), dried over anhydrous sodium sulfate, filtered and concentrated. The residue was purified by flash silica gel chromatography (ISCO®; 20 g SepaFlash® Silica Flash Column, Eluent of 0˜70% Ethylacetate/Petroleum ethergradient @ 20 mL/min). The cut fraction was concentrated under reduced pressure to give methyl N-[(5-bromo-4-methyl-thiazol-2-yl)methyl]carbamate 16a (440 mg, 1.66 mmol, 34.37% yield) was obtained as a solid. ¹H NMR (400 MHz, CHLOROFORM-d) 7.26 (s, 1H), 5.46-5.22 (m, 1H), 4.55 (br d, J=6.0 Hz, 2H), 3.72 (s, 3H), 2.36 (s, 3H).

### Step 2: Synthesis of methyl N-[[5-[2-[4,7-difluoro-1-(2-trimethylsilylethoxymethyl)benzotriazol-5-yl]thiazol-4-yl]-4-methyl-thiazol-2-yl]methyl]carbamate (16b)

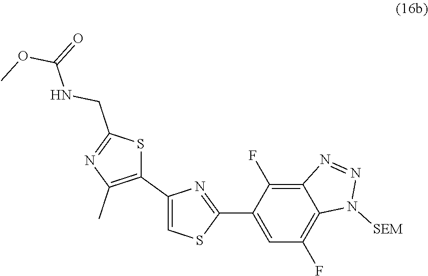 [SMILES: COC(=O)NCc1nc(C)c(-c2csc(-c3cc(F)c4c(nnn4COCC[Si](C)(C)C)c3F)n2)s1]

A mixture of Pd(OAc)₂ (19.07 mg, 84.94 umol, 0.1 eq) and bis(1-adamantyl)-butyl-phosphane (60.91 mg, 169.88 umol, 0.2 eq) in Tol. (8 mL) was stirred at 20° C. for 0.5 h under N₂. The above mixture was added to a mixture of 2-[[5-(4-bromothiazol-2-yl)-4,7-difluoro-benzotriazol-1-yl]methoxy]ethyl-trimethyl-silane (6, Key Intermediate F, 380 mg, 849.40 umol, 1 eq), methyl N-[(5-bromo-4-methyl-thiazol-2-yl)methyl]carbamate 16a (270.24 mg, 1.02 mmol, 1.2 eq), 4,4,5,5-tetramethyl-2-(4,4,5,5-tetramethyl-1,3,2-dioxaborolan-2-yl)-1,3,2-dioxaborolane (862.79 mg, 3.40 mmol, 4 eq) and CsF (387.07 mg, 2.55 mmol, 93.95 uL, 3 eq) in MeOH (32 mL). The mixture was taken up into a microwave tube. The sealed tube was heated at 80° C. for 1 h under microwave under N₂. TLC indicated one new spot formed. The reaction mixture was filtered and concentrated under reduced pressure to give a residue. The residue was purified with flash silica gel chromatography (ISCO®; 20 g SepaFlash® Silica Flash Column, Eluent of 0˜100% PE/EA gradient @ 75 mL/min) to give methyl N-[[5-[2-[4,7-difluoro-1-(2-trimethylsilylethoxymethyl)benzotriazol-5-yl]thiazol-4-yl]-4-methyl-thiazol-2-yl]methyl]carbamate 16b (110 mg, 199.03 umol, 23.43% yield) as a solid.

Step 3: Synthesis of methyl N-[[5-[2-(4,7-difluoro-1H-benzotriazol-5-yl)thiazol-4-yl]-4-methyl-thiazol-2-yl]methyl]carbamate (16). To a solution of methyl N-[[5-[2-[4,7-difluoro-1-(2-trimethylsilylethoxymethyl)benzotriazol-5-yl]thiazol-4-yl]-4-methyl-thiazol-2-yl]methyl]carbamate 16b (100 mg, 180.93 umol, 1 eq) in DCM (5 mL) was added TFA (3.08 g, 27.01 mmol, 2.00 mL, 149.29 eq) at 0° C., and the mixture was stirred at 20° C. for 2 h. LCMS showed desired MS was detected. The reaction mixture was concentrated under reduced pressure to give a residue and purified by prep-HPLC (column: Welch Xtimate C18 150*30 mm*5 um; mobile phase: [water (0.05% ammonia hydroxide v/v)-ACN]; B %: 10%-40%, 11.5 min), and was further purified by prep-HPLC (column: Welch Xtimate C18 150*30 mm*5 um;mobile phase: [water (0.05% ammonia hydroxide v/v)-ACN]; B %: 10%-40%, 11.5 min) to give methyl N-[[5-[2-(4,7-difluoro-1H-benzotriazol-5-yl)thiazol-4-yl]-4-methyl-thiazol-2-yl]methyl]carbamate 16 (26 mg, 61.55 umol, 34.02% yield, 99.67% purity) as a solid. ¹H NMR (400 MHz, DMSO-d₆) δ=8.16-8.04 (m, 2H), 7.92 (dd, J=4.4, 10.5 Hz, 1H), 4.44 (d, J=6.1 Hz, 2H), 3.62 (s, 3H), 2.62 (s, 3H), HPLC (99.67% purity); ES-MS m/z 423.0 [M+H]⁺.

Example S17. Compounds 17-212 were prepared in accordance with the procedures described in Examples S7-S16 using suitable reagents and modifications as would be known by a person of skill in the art.

Compounds described herein may be prepared according to the methods and examples as described herein and were characterized using standard techniques known in the art, such as ¹H NMR and mass spectra.

### BIOLOGICAL EXAMPLES

### Example B1. BPGM Synthase Assay

Materials

The following materials were used in this assay: deionized water; Bisphosphoglycerate mutase (BPGM; Standard Buffer: 50 mM Tris, 0.01% Tween 20, pH 7.4); BPGM Reaction Buffer (50 mM Tris, 0.01% Tween 20, 3.23 mM KH₂PO₄, pH 7.4); dithiothreitol (DTT; Stock solution: 100 mM in Standard Buffer); 384-Well Black Assay Plate (Corning, P/N 3575); DMSO; BPGM Stock Solution (500 nM in BPGM Standard Buffer); Glyceraldehyde 3-phosphate dehydrogenase (GAPDH; Stock Solution: 40 U/mL in 20 mM Tris-HCL Buffer with 20% Glycerol, 1 mM EDTA, 1 mM DTT); Glyceraldehyde 3-phosphate (GAP; Stock Solution: 20 mM in BPGM Standard Buffer); 3-PG Stock Solution (500 μM in BPGM Standard Buffer); Nicotinamide adenine dinucleotide (NAD; Stock Solution: 50 mM in BPGM Standard Buffer); Koningic Acid (1 mM Stock in DMSO); Resazurin Stock Solution (10 mM in DMSO); Diaphorase Stock Solution (40 U/mL in BPGM Standard Buffer); DTT Reaction Buffer (1 mM DTT in BPGM Reaction Buffer); Enzyme Solution (5 nM BPGM, 0.4 U/mL GAPDH in DTT Reaction Buffer); GAPDH Solution (0.4 U/mL GAPDH in DTT Reaction Buffer); Substrate Solution (2 mM NAD, 20 μM 3PG, 800 μM GAP in DTT Reaction Buffer); and Resazurin Solution (5 U/mL Diaphorase, 500 μM Resazurin in BPGM Standard Buffer).

Procedures

To the assay plate was added Enzyme Solution (20 uL) to columns 2-22 and 24 of the assay plate. To columns 1 and 23 was added GAPDH Solution (20 uL). A separate compound plate was prepared at the desired concentrations and all wells were normalized with 5% DMSO. The compound plate was incubated for 30 minutes at RT. Next, the substrate solution (20 μL) was dispensed to all reaction wells and the plate was incubated for 60 minutes at RT. Koningic acid (1 μL) was added and the plate was incubated for 10 minutes at RT. During this time period, the NADH fluorescence was read at ex: 360/em: 460. Next, Resazurin Solution (10 μL) was added to all reaction wells and the plate was incubated for 30 minutes at RT. The resorufin fluorescence was then read at ex:544/em:590.

The results of the BPGM synthase assay for select exemplary compounds are shown in Table 3.

TABLE 3
hBPGM Synthase IC₅₀ Values for Exemplary Compounds.

| Compound No. | hBPGM Synthase IC₅₀ (nM) |
|---|---|
| 7 | 92 |
| 8 | 21 |
| 9 | 19 |
| 10 | 159 |
| 11 | 511 |
| 12 | 256 |
| 13 | 169 |
| 14 | 1010 |
| 15 | 138 |
| 16 | 50 |

### Example B2. BPGM Phosphatase Assay

Materials

The following materials were used in this assay: Tris buffer (50 mM Tris, 0.01% Tween 20, pH 7.4); cyclohexylammonium salt of 2,3-BPG (stock solution of 500 mM in ultapure distilled water; human Bisphosphoglycerate mutase (hBPGM; stock solution of 110 μM in Standard Buffer); 3-Phosphoglyceric acid (3-PG; stock solution of 100 mM in ultrapure distilled water); 384-Well Polystyrene Plates (Fisherbrand™, Catalog No.: 12-566-625); BIOMOL GREEN REAGENT (Enzo life sciences, BML-AK111-0250); and 1 mM DMSO stock of exemplary compounds.

Procedures

A 3-PG enzyme mix solution (final enzyme concentration 62.5 nM, 3-PG concentration at 50 M, 14.5 uL) was added to onto a 384-well clear plate. Solutions of the test compounds were then added to the plate and the DMSO content was normalized across the plate. The plate was incubated at RT for 15 min. Next, the substrate 2,3-BPG (5 μL) was added to all the wells, and the plate was sealed and incubated for 24 hr at RT. After incubation, BIOMOL green dye (40 μL) was added to all the wells, the plate was shaken to mix, and the plate was incubated at RT for 20-30 min. Absorbance measurements were recorded at 620 nm.

Results

The results of the BPGM phosphatase assay for select exemplary compounds are shown in Table 4.

TABLE 4
hBPGM Phosphatase EC₅₀ Values for Exemplary Compounds.

| Compound No. | hBPGM Phosphatase EC₅₀ (nM) |
|---|---|
| 7 | 55 |
| 8 | 40 |
| 10 | 310 |
| 11 | 190 |
| 13 | 55 |
| 14 | 570 |
| 15 | 170 |

### Example B3. Human Cell-Based Assay

Materials

The following materials were used in this assay: DMSO; Hemox buffer (30 mM TES, 135 mM NaCl, 5 mM KCl, pH 7.4); Inosine (100 mM in hemox buffer); Glucose (1 M in water); human red blood cells; LC/MS Optima grade methanol; LC/MS Optima grade water; and Incubation buffer (30 mM TES, 135 mM NaCl, 5 mM KCl, 50 mM inosine, 20 mM glucose pH 7.4).

Procedures

Test compounds were loaded onto a 96-well plate (final concentration of DMSO was 1%). 10 mL whole blood was resuspended with an equivalent amount of 2× incubation buffer to make 25% het & 50% plasma solution. The final solution contains 10 mM glucose and 25 mM inosine. The blood suspension (100 uL) was added to the wells of the plate. The plate was covered with an adhesive foil cover and plastic lid, and incubated at 37° C. for 24 h. The samples were extracted through an SPE column and the metabolites (2,3-BPG, ATP) were analyzed by LC/MS.

Results

The results of the cell-based assay for a select exemplary compound is shown in Table 5.

TABLE 5
hRBC EC₅₀ Values for Exemplary Compounds.

| Compound No. | hRBC EC₅₀ (μM) |
|---|---|
| 16 | 22.5 |

Additional results from the hBPGM synthase and hBPGM phosphate assays as described herein are shown in Table 6.

TABLE 6
Additional Assay Results

| | huBPGM: phosphatase | huBPGM: synthase |
|---|---|---|
| Compound No. | Enzyme EC50 (M) | Enzyme IC50 (M) |
| 7 | 5.50E−08 | 9.20E−08 |
| 8 | 4.00E−08 | 1.70E−08 |
| 9 | | 1.90E−08 |
| 10 | 3.10E−07 | 1.30E−07 |
| 11 | 1.90E−07 | 5.10E−07 |
| 12 | | 2.60E−07 |
| 13 | 3.20E−08 | 1.70E−07 |
| 15 | 1.70E−07 | 1.40E−07 |
| 24 | | 2.50E−07 |
| 25 | | 4.40E−06 |
| 26 | | 1.10E−07 |
| 27 | | 1.50E−06 |
| 28 | | 1.60E−06 |
| 29 | | 5.70E−06 |
| 30 | | 2.00E−07 |
| 31 | | 3.50E−08 |
| 32 | | 7.60E−08 |
| 33 | | 2.50E−07 |
| 34 | | 2.50E−07 |
| 35 | | 6.00E−07 |
| 36 | | 1.30E−06 |
| 37 | | 1.30E−05 |
| 38 | | 3.60E−08 |
| 39 | | 4.10E−08 |
| 40 | | 4.40E−08 |
| 41 | | 7.50E−08 |
| 42 | | 4.10E−06 |
| 43 | | 1.40E−08 |
| 44 | | 2.50E−08 |
| 45 | | 4.80E−08 |
| 46 | | 1.30E−07 |
| 47 | | 2.50E−08 |
| 48 | | 3.10E−08 |
| 49 | | 4.70E−08 |
| 50 | | 7.40E−08 |
| 51 | | 2.80E−07 |
| 52 | | 3.90E−07 |
| 53 | | 8.90E−07 |
| 54 | | 9.10E−08 |
| 55 | | 2.20E−07 |
| 56 | | 2.60E−07 |
| 57 | | 2.90E−07 |
| 58 | | 1.90E−06 |
| 59 | | 2.00E−08 |
| 60 | | 5.50E−08 |
| 61 | | 2.20E−07 |
| 62 | | 3.50E−07 |
| 63 | | 1.40E−06 |
| 64 | | 4.30E−08 |
| 65 | | 2.80E−07 |
| 66 | | 1.60E−08 |
| 67 | | 5.00E−08 |
| 68 | | 2.00E−07 |
| 69 | | 7.90E−08 |
| 70 | | 2.00E−07 |
| 71 | | 3.00E−07 |
| 72 | | 5.10E−07 |
| 73 | | >2.5E−05 |
| 74 | | 2.90E−07 |
| 75 | | 4.40E−07 |
| 76 | | 1.10E−06 |
| 77 | | 1.30E−06 |
| 78 | | 1.50E−06 |
| 79 | | 2.40E−06 |
| 80 | | 2.80E−06 |
| 81 | | 3.00E−06 |
| 82 | | 3.60E−06 |
| 83 | | >2.5E−05 |
| 84 | | >2.5E−05 |
| 85 | | 2.10E−08 |
| 86 | | 1.40E−07 |
| 87 | | 3.80E−07 |
| 88 | | 2.40E−08 |
| 89 | | 2.80E−07 |
| 90 | | 3.40E−07 |
| 91 | | 6.40E−07 |
| 92 | | 2.30E−06 |
| 93 | | 9.00E−06 |
| 94 | | >2.5E−05 |
| 95 | 2.50E−08 | 2.50E−08 |
| 96 | 1.00E−06 | 1.60E−06 |
| 97 | 6.90E−06 | 1.80E−06 |
| 98 | 3.80E−07 | 2.20E−06 |
| 99 | 7.80E−06 | 4.70E−06 |
| 100 | 5.70E−08 | 3.10E−07 |
| 101 | 3.90E−08 | 4.50E−07 |
| 102 | 4.50E−08 | 6.20E−07 |
| 103 | >2.5E−05 | 9.30E−07 |
| 104 | >2.5E−05 | 2.30E−06 |
| 105 | | 2.20E−07 |
| 106 | | 3.30E−07 |
| 107 | | 3.20E−06 |
| 108 | | 1.40E−05 |
| 109 | | 1.60E−07 |
| 110 | | 1.60E−07 |
| 111 | | 5.70E−07 |
| 112 | | 6.50E−07 |
| 113 | | 5.30E−07 |
| 114 | | 1.20E−06 |
| 115 | | 4.40E−06 |
| 116 | | 6.50E−06 |
| 117 | | >2.5E−05 |
| 118 | | 2.80E−08 |
| 119 | | 2.90E−06 |
| 120 | | 1.50E−06 |
| 121 | | 3.30E−06 |
| 122 | | 3.30E−08 |
| 123 | | 1.60E−06 |
| 124 | | 6.30E−06 |
| 125 | 2.70E−06 | 2.30E−08 |
| 126 | >2.5E−05 | 2.10E−08 |
| 127 | 3.80E−07 | 4.30E−07 |
| 128 | | 6.70E−06 |
| 129 | 4.00E−08 | 8.00E−09 |
| 130 | 1.00E−05 | 1.50E−08 |
| 131 | 5.70E−07 | 5.10E−07 |
| 132 | | 8.10E−06 |
| 133 | | 4.40E−06 |
| 134 | | >2.5E−05 |
| 135 | 2.40E−07 | 1.60E−07 |
| 136 | 7.00E−08 | 2.00E−07 |
| 137 | 2.00E−07 | 2.30E−07 |
| 138 | >2.5E−05 | 9.60E−07 |
| 139 | >2.5E−05 | >2.5E−05 |
| 140 | 3.00E−08 | |
| 141 | 5.70E−08 | 5.40E−08 |
| 142 | 4.00E−08 | 2.60E−07 |
| 144 | 4.00E−08 | 3.30E−07 |
| 145 | 3.00E−08 | 2.50E−07 |
| 146 | | 1.30E−05 |
| 147 | 4.90E−07 | 5.50E−07 |
| 148 | 2.50E−07 | 1.20E−06 |
| 149 | | 4.50E−06 |
| 150 | | >9.9E−05 |
| 151 | 2.00E−05 | 1.50E−06 |
| 152 | 3.00E−08 | 4.00E−08 |
| 153 | 6.00E−08 | 8.10E−08 |
| 154 | 4.90E−08 | 1.10E−07 |
| 155 | 4.00E−08 | 4.40E−08 |
| 156 | 1.50E−07 | 2.00E−07 |
| 157 | 2.40E−07 | 1.70E−07 |
| 158 | 5.30E−07 | 2.40E−07 |
| 159 | 1.80E−06 | 2.90E−07 |
| 160 | 5.20E−07 | 4.60E−07 |
| 161 | | >2.5E−05 |
| 162 | | >2.5E−05 |
| 163 | 2.60E−07 | 1.70E−07 |
| 164 | 5.50E−07 | 6.80E−07 |
| 165 | 1.10E−07 | 9.20E−08 |
| 166 | 1.70E−07 | 1.10E−07 |
| 167 | 7.50E−08 | 1.30E−07 |
| 168 | 1.30E−07 | 2.20E−07 |
| 169 | 3.30E−07 | 5.60E−07 |
| 170 | 8.80E−07 | 1.10E−06 |
| 171 | 1.90E−06 | 8.10E−07 |
| 172 | 2.40E−07 | 2.80E−07 |
| 173 | >2.5E−05 | 1.50E−06 |
| 174 | | 5.00E−06 |
| 175 | 3.00E−08 | 7.90E−08 |
| 176 | 2.00E−08 | 3.40E−06 |
| 177 | 2.50E−07 | 4.40E−07 |
| 178 | 1.70E−06 | 1.70E−06 |
| 179 | | |
| 180 | | >2.5E−05 |
| 181 | 7.90E−07 | 3.00E−07 |
| 182 | | 1.90E−06 |
| 183 | | >2.5E−05 |
| 184 | | 2.50E−07 |
| 185 | | 8.90E−07 |
| 186 | 3.50E−07 | 2.40E−07 |
| 187 | 4.80E−07 | 3.00E−07 |
| 188 | 1.40E−07 | 8.40E−08 |
| 189 | 3.10E−07 | 9.80E−07 |
| 190 | 3.30E−06 | 4.20E−06 |
| 191 | 1.60E−06 | 1.10E−06 |
| 192 | 4.90E−07 | 1.00E−06 |
| 193 | | 2.30E−05 |
| 194 | 3.70E−07 | 8.20E−07 |
| 196 | 4.90E−06 | 1.60E−06 |
| 197 | 4.00E−07 | 3.10E−06 |
| 198 | | 5.20E−06 |
| 199 | | 6.50E−06 |
| 200 | | 1.20E−05 |
| 201 | 1.80E−07 | 4.10E−07 |
| 202 | | >2.5E−05 |
| 203 | 3.60E−06 | 6.10E−06 |
| 204 | | >2.5E−05 |
| 205 | 4.40E−07 | 3.40E−07 |
| 206 | 1.20E−06 | 1.40E−06 |
| 207 | 8.20E−06 | 6.50E−06 |
| 208 | 1.60E−05 | 3.10E−06 |
| 209 | | 3.30E−06 |
| 210 | | 7.50E−06 |
| 211 | | 7.40E−07 |
| 212 | | 2.60E−06 |
| 213 | 6.00E−08 | 1.60E−07 |
| 214 | 9.60E−08 | 1.80E−07 |
| 215 | | >2.5E−05 |
| 216 | | 5.40E−06 |
| 217 | | 1.20E−07 |
| 218 | | 4.10E−07 |
| 219 | | 5.80E−08 |
| 220 | | 2.70E−08 |
| 221 | | 1.70E−07 |
| 222 | | >2.5E−05 |
| 223 | | 8.40E−07 |
| 224 | | 3.00E−08 |
| 225 | | 2.10E−07 |
| 226 | | 8.20E−08 |
| 227 | | 2.10E−08 |
| 228 | | >2.5E−05 |
| 229 | | >2.5E−05 |
| 230 | | >2.5E−05 |
| 231 | | 8.70E−08 |
| 232 | | 2.20E−06 |
| 233 | | 3.90E−06 |
| 234 | | 1.30E−06 |
| 235 | | 1.30E−07 |
| 236 | | 1.10E−07 |
| 237 | | 2.20E−08 |
| 238 | | 4.90E−08 |
| 239 | 4.80E−07 | 3.00E−07 |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

1. A compound of Formula (I): 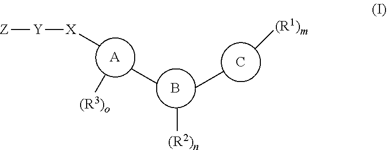
or a pharmaceutically acceptable salt thereof, wherein:
Ring A is a 5-membered heteroarylene containing 1-3 heteroatoms selected from N and S;
Ring B is phenylene or a 5- to 6-membered heteroarylene containing 1-3 heteroatoms selected from N and S;
Ring C is a fused bicyclic 9- to 10-membered heteroaryl or heterocyclyl containing 1-4 heteroatoms selected from N and O, a 5- to 6-membered heteroaryl containing 1-4 nitrogen atoms, or phenyl substituted by one R⁴;
each R¹ is independently —OH, halo, oxo, C₁-C₆ alkyl, C₁-C₆ haloalkyl, —CN, —N(H)C(O)R⁶, —N(H)SO₂R⁶, —N(H)SO₂(C₆H₅), —SO₂NR⁶R⁷, or —C(O)N(H)SO₂R⁶;
each R² is independently C₁-C₆ alkyl, C₁-C₆ haloalkyl, or halo;
each R³ is independently C₁-C₆ alkyl, C₁-C₆ haloalkyl, or C₃-C₆ cycloalkyl;
R⁴ is —OH, —OP(O)(OH)₂, —NO₂, —C(O)N(H)SO₂R⁵, 5- to 6-membered heteroaryl or heterocyclyl, or —C(O)N(H)-(5- to 6-membered heteroaryl or heterocyclyl),
wherein the heteroaryl and heterocyclyl contain 1-4 heteroatoms selected from N, O, and S, and wherein the heterocyclyl is optionally substituted by 1-2 oxo groups;
m is 0-4;
n is 0-2;
o is 0-2;
R⁵ is phenyl or C₁-C₆ alkyl;
X is —CR⁶R⁷— or a bond;
Y is —O—, —N(H)—, or a bond;
each R⁶ and R⁷ is independently H or C₁-C₆ alkyl;
Z is Z¹ or Z²;
Z¹ is H, C₁-C₆ alkyl, —C(O)(C₁-C₆ alkyl), —CO₂(C₁-C₆ alkyl), —C(O)NR⁶R⁷, —C(O)NR⁶(C₁-C₆ alkylene)NR⁶R⁷, —CR⁶R⁷C(O)NR⁶R⁷, —CR⁶R⁷NR⁶R⁷, —C(O)(C₁-C₆ alkylene)NR⁶R⁷, —NR⁶C(O)(C₁-C₆ alkyl), —NR⁶R⁷, —(C₁-C₆ alkyl)-CO₂H, —(C₁-C₆ alkyl)-OH, or —C(NR⁶R⁷)═N—CN,
wherein C₁-C₆ alkylene is optionally substituted by 1-6 halo, C₁-C₆ alkyl, or C₁-C₆ haloalkyl;
Z² is 5- to 6-membered heteroaryl, —C(O)(5- to 6-membered heteroaryl), —CH₂C(O)(5- to 6-membered heteroaryl), 4- to 6-membered heterocyclyl, —C(O)(4- to 6-membered heterocyclyl), or —CH₂C(O)(4- to 6-membered heterocyclyl), wherein the heteroaryl and heterocyclyl contain 1-3 heteroatoms selected from N and O, and wherein the heteroaryl and heterocyclyl are optionally substituted by 1-3 R⁸;
each R⁸ is independently halo, C₁-C₆ alkyl, C₁-C₆ haloalkyl, —NR⁶R⁷, —OH, oxo, —CO₂H, —O(C₁-C₆ alkyl), —CH₂—O(C₁-C₆ alkyl), or —(C₁-C₆ alkyl)-OH.

2. The compound of claim 1, wherein the compound is of Formula (I-a): 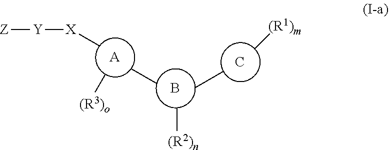
or a pharmaceutically acceptable salt thereof, wherein:
Ring A is thiazolylene;
Ring B is phenylene or a 5- to 6-membered heteroarylene containing 1-3 heteroatoms selected from N and S;
Ring C is a fused bicyclic 9- to 10-membered heteroaryl or heterocyclyl containing 1-4 heteroatoms selected from N and O, a 5- to 6-membered heteroaryl containing 1-4 nitrogen atoms, or phenyl substituted by one R⁴;
each R¹ is independently —OH, halo, oxo, C₁-C₆ alkyl, C₁-C₆ haloalkyl, —CN, —N(H)C(O)R⁶, —N(H)SO₂R⁶, —SO₂NR⁶R⁷, or —C(O)N(H)SO₂R⁶;
each R² is independently C₁-C₆ alkyl, C₁-C₆ haloalkyl, or halo;
each R³ is independently C₁-C₆ alkyl, C₁-C₆ haloalkyl, or C₃-C₆ cycloalkyl;
R⁴ is —OH, —OP(O)(OH)₂, —NO₂, —C(O)N(H)SO₂R⁵, 5- to 6-membered heteroaryl or heterocyclyl, or —C(O)N(H)-(5- to 6-membered heteroaryl or heterocyclyl),
wherein the heteroaryl and heterocyclyl contain 1-4 heteroatoms selected from N, O, and S, and wherein the heterocyclyl is optionally substituted by 1-2 oxo groups;
m is 0-4;
n is 0-2;
o is 0-2;
R⁵ is phenyl or C₁-C₆ alkyl;
X is —CR⁶R⁷—;
Y is —O—, —N(H)—, or a bond;
each R⁶ and R⁷ is independently H or C₁-C₆ alkyl;
Z is Z²;
Z² is 5- to 6-membered heteroaryl, —C(O)(5- to 6-membered heteroaryl), —CH₂C(O)(5- to 6-membered heteroaryl), 4- to 6-membered heterocyclyl, —C(O)(4- to 6-membered heterocyclyl), or —CH₂C(O)(4- to 6-membered heterocyclyl), wherein the heteroaryl and heterocyclyl contain 1-3 heteroatoms selected from N and O, and wherein the heteroaryl and heterocyclyl are optionally substituted by 1-3 R⁸; and
each R⁸ is independently halo, C₁-C₆ alkyl, C₁-C₆ haloalkyl, —NR⁶R⁷, —OH, oxo, —CO₂H, —O(C₁-C₆ alkyl), —CH₂—O(C₁-C₆ alkyl), or —(C₁-C₆ alkyl)-OH.

3. The compound of claim 1, wherein the compound is of Formula (I-a1): 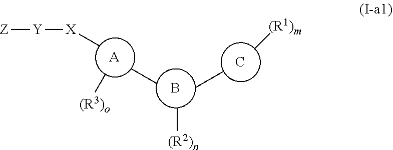
or a pharmaceutically acceptable salt thereof, wherein:
Ring A is thiazolylene;
Ring B is a 5- to 6-membered heteroarylene containing 1-3 heteroatoms selected from N and S;
Ring C is a fused bicyclic 9- to 10-membered heteroaryl or phenyl substituted by one R⁴;
each R¹ is independently —OH, halo, oxo, C₁-C₆ alkyl, C₁-C₆ haloalkyl, —CN, —N(H)C(O)R⁶, —N(H)SO₂R⁶, —SO₂NR⁶R⁷, or —C(O)N(H)SO₂R⁶;
each R² is independently C₁-C₆ alkyl, C₁-C₆ haloalkyl, or halo;
each R³ is independently C₁-C₆ alkyl, C₁-C₆ haloalkyl, or C₃-C₆ cycloalkyl;
R⁴ is —OH, —OP(O)(OH)₂, —NO₂, —C(O)N(H)SO₂R⁵, 5- to 6-membered heteroaryl or heterocyclyl, or —C(O)N(H)-(5- to 6-membered heteroaryl or heterocyclyl),
wherein the heteroaryl and heterocyclyl contain 1-4 heteroatoms selected from N, O, and S, and wherein the heterocyclyl is optionally substituted by 1-2 oxo groups;
m is 0-4;
n is 0-2;
o is 0-2;
R⁵ is phenyl or C₁-C₆ alkyl;
X is —CR⁶R⁷—;
Y is —O—, —N(H)—, or a bond;
each R⁶ and R⁷ is independently H or C₁-C₆ alkyl;
Z is Z²;
Z² is 5- to 6-membered heteroaryl, —C(O)(5- to 6-membered heteroaryl), —CH₂C(O)(5- to 6-membered heteroaryl), 4- to 6-membered heterocyclyl, —C(O)(4- to 6-membered heterocyclyl), or —CH₂C(O)(4- to 6-membered heterocyclyl), wherein the heteroaryl and heterocyclyl contain 1-3 heteroatoms selected from N and O, and wherein the heteroaryl and heterocyclyl are optionally substituted by 1-3 R⁸; and
each R⁸ is independently halo, C₁-C₆ alkyl, C₁-C₆ haloalkyl, —NR⁶R⁷, —OH, oxo, —CO₂H, —O(C₁-C₆ alkyl), —CH₂—O(C₁-C₆ alkyl), or —(C₁-C₆ alkyl)-OH.

4. The compound of claim 1, or a pharmaceutically acceptable salt thereof, wherein Ring A is thiazolylene.

5. The compound of any one of claims 1-3, or a pharmaceutically acceptable salt thereof, wherein each R³ is independently C₁-C₃ alkyl or C₃-C₆ cycloalkyl.

6. The compound of claim 4, or a pharmaceutically acceptable salt thereof, wherein each R³ is independently —CH₃ or cyclopropyl.

7. The compound of any one of claims 1-5, or a pharmaceutically acceptable salt thereof, wherein o is 0 or 1.

8. The compound of any one of claims 1-2 or 4-7, or a pharmaceutically acceptable salt thereof, wherein Ring B is phenylene, pyridinylene, thiazolylene, pyrazolylene, pyrimidinylene, or thienylene.

9. The compound of any one of claims 1-8, or a pharmaceutically acceptable salt thereof, wherein each R² is independently C₁-C₃ alkyl or halo.

10. The compound of any one of claims 1-9, or a pharmaceutically acceptable salt thereof, wherein n is 0 or 1.

11. The compound of any one of claims 1-2 and 4-11, or a pharmaceutically acceptable salt thereof, wherein:
Ring C is a fused bicyclic 9-membered heteroaryl or heterocyclyl containing 1-4 heteroatoms selected from N and O, a 5- to 6-membered heteroaryl containing 1-4 nitrogen atoms, or phenyl substituted by one R⁴.

12. The compound of any one of claims 1-11, or a pharmaceutically acceptable salt thereof, wherein:
R⁴ is —OH, —OP(O)(OH)₂, —NO₂, —C(O)N(H)SO₂R⁵, 5- to 6-membered heteroaryl or heterocyclyl, or —C(O)N(H)-(5- to 6-membered heteroaryl or heterocyclyl),
wherein the heteroaryl and heterocyclyl contain 1-4 heteroatoms selected from N, O, and S, and wherein the heterocyclyl is optionally substituted by 1-2 oxo groups; and
R⁵ is phenyl or C₁-C₃ alkyl.

13. The compound of any one of claims 1-2 and 4-12, or a pharmaceutically acceptable salt thereof, wherein:
Ring C—(R¹)ₘ is selected from:

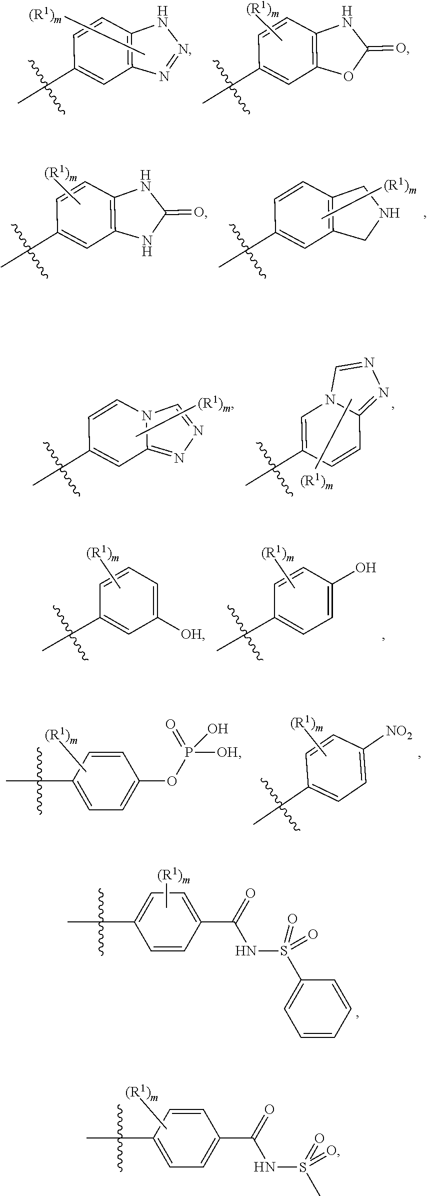

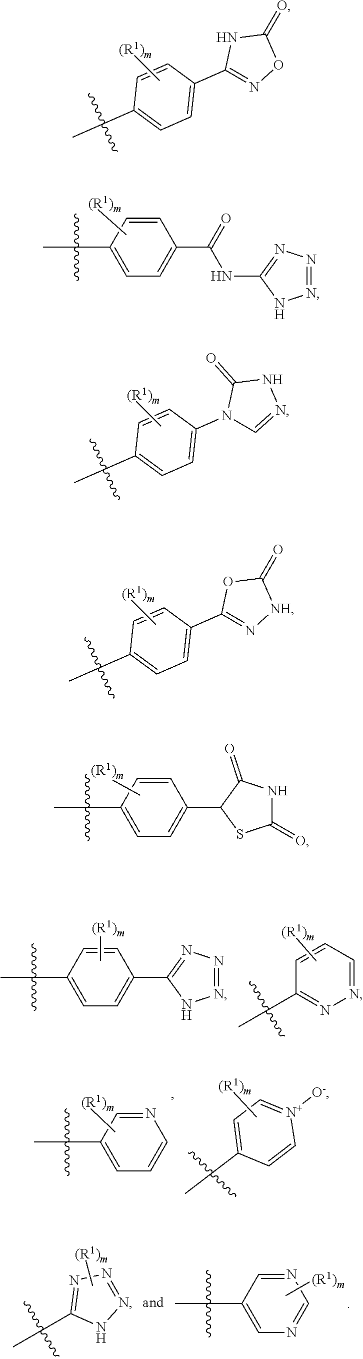

14. The compound of any one of claims 1-13, or a pharmaceutically acceptable salt thereof, wherein:
each R¹ is independently —OH, halo, oxo, C₁-C₃ alkyl, C₁-C₃ haloalkyl, —CN, —N(H)C(O)R⁶, —N(H)SO₂R⁶, —SO₂NR⁶R⁷, or —C(O)N(H)SO₂R⁶; and
each R⁶ and R⁷ is independently H or C₁-C₃ alkyl.

15. The compound of any one of claims 1-13, or a pharmaceutically acceptable salt thereof, wherein:
each R¹ is independently —OH, F, oxo, —CH₃, —CN, —N(H)C(O)CH₃, —N(H)SO₂(C₆H₅), —N(H)SO₂CH₃, —SO₂NH₂, or —C(O)N(H)SO₂CH₃.

16. The compound of any one of claims 1-15, or a pharmaceutically acceptable salt thereof, wherein m is 0-3.

17. The compound of any one of claims 1 and 4-16, or a pharmaceutically acceptable salt thereof, wherein X is —CR⁶R⁷—.

18. The compound of any one of claims 1-3 and 17, or a pharmaceutically acceptable salt thereof, wherein R⁶ and R⁷ are each H.

19. The compound of any one of claims 1 and 4-16, or a pharmaceutically acceptable salt thereof, wherein X is a bond.

20. The compound of any one of claims 1-19, or a pharmaceutically acceptable salt thereof, wherein Y is —O—.

21. The compound of any one of claims 1-19, or a pharmaceutically acceptable salt thereof, wherein Y is —N(H)— or a bond.

22. The compound of any one of claims 1 and 4-21, or a pharmaceutically acceptable salt thereof, wherein Z is Z¹.

23. The compound of claim 22, or a pharmaceutically acceptable salt thereof, wherein:
Z¹ is H, C₁-C₃ alkyl, —C(O)(C₁-C₃ alkyl), —CO₂(C₁-C₃ alkyl), —C(O)NR⁶R⁷, —C(O)NR⁶(C₁-C₃ alkylene)NR⁶R⁷, —CR⁶R⁷C(O)NR⁶R⁷, —CR⁶R⁷NR⁶R⁷, —C(O)(C₁-C₃ alkylene)NR⁶R⁷, —NR⁶C(O)(C₁-C₃ alkyl), —NR⁶R⁷, —(C₁-C₃ alkyl)-CO₂H, —(C₁-C₃ alkyl)-OH, or —C(NR⁶R⁷)═N—CN, wherein C₁-C₃ alkylene is optionally substituted by 1-2 halo, C₁-C₃ alkyl, or C₁-C₃ haloalkyl; and
each R⁶ and R⁷ is independently H or C₁-C₃ alkyl.

24. The compound of claim 22, or a pharmaceutically acceptable salt thereof, wherein:
Z¹ is H, —CO₂CH₃, —CO₂C(CH₃)₃, —C(O)CH₃, —C(O)C(CH₃)₃, —C(N(CH₃)₂)═N—CN, —C(O)N(CH₃)₂, —CH₃, —CH₂CH₃, —CH₂C(CH₃)₃, —CH₂CO₂H, —CH₂C(CH₃)₂OH, —C(O)N(CH₃)CH₂CH₂N(CH₃)₂, —CH₂C(O)N(CH₃)₂, —CH(CH₃)C(O)N(CH₃)₂, —N(CH₃)₂, —N(CH₃)C(O)CH₃, or —C(O)CH(CH₃)CH₂N(CH₃)₂.

25. The compound of any one of claims 1-21, or a pharmaceutically acceptable salt thereof, wherein Z is Z².

26. The compound of claim 24, or a pharmaceutically acceptable salt thereof, wherein:
Z² is 5- to 6-membered heteroaryl, —C(O)(5- to 6-membered heteroaryl), —CH₂C(O)(5- to 6-membered heteroaryl), 4- to 6-membered heterocyclyl, —C(O)(4- to 6-membered heterocyclyl), or —CH₂C(O)(4- to 6-membered heterocyclyl), wherein the heteroaryl and heterocyclyl contain 1-2 heteroatoms selected from N and O, and wherein the heteroaryl and heterocyclyl are optionally substituted by 1-3 R⁸.

27. The compound of claim 25, or a pharmaceutically acceptable salt thereof, wherein:
Z² is pyrimidinyl, pyridazinyl, pyrrolidinyl, piperidinyl, pyridazinyl, —C(O)(tetrahydropyranyl), —C(O)(pyrrolidinyl), —C(O)(pyridazinyl), —C(O)(piperidinyl), —C(O)(azetidinyl), —C(O)(pyrazolyl), —C(O)(piperazinyl), —CH₂C(O)(pyrrolidinyl), —CH₂C(O)(piperidinyl), or —CH₂C(O)(piperazinyl), wherein the heteroaryl and heterocyclyl are optionally substituted by 1-3 R⁸.

28. The compound of any one of claims 1-21 and 25-27, or a pharmaceutically acceptable salt thereof, wherein:
each R⁸ is independently halo, C₁-C₃ alkyl, C₁-C₃ haloalkyl, —NR⁶R⁷, —OH, oxo, —CO₂H, —O(C₁-C₃ alkyl), —CH₂—O(C₁-C₃ alkyl), or —(C₁-C₃ alkyl)-OH; and
each R⁶ and R⁷ is independently H or C₁-C₃ alkyl.

29. The compound of claim 28, or a pharmaceutically acceptable salt thereof, wherein:
each R⁸ is independently —CH₃, —CH₂CH₃, —NH₂, —OH, oxo, —N(CH₃)₂, —OCH₃, —CH₂OCH₃, —CF₃, —CH₂CH₂OH, —CO₂H, or Cl.

30. The compound of claim 1, or a pharmaceutically acceptable salt thereof, wherein Z—Y—X— is:

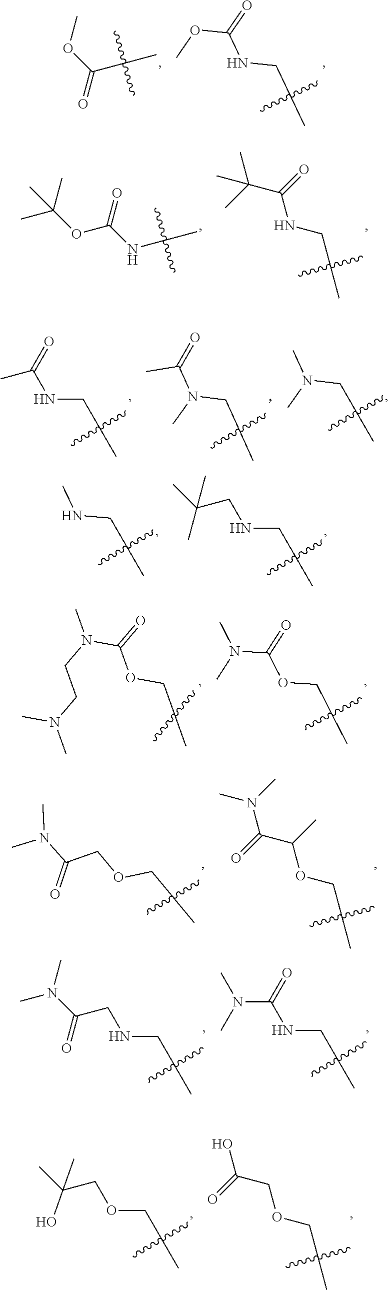

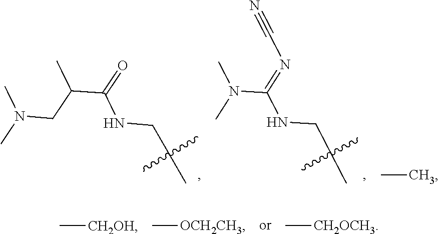

31. The compound of claim 1, or a pharmaceutically acceptable salt thereof, wherein Z—Y—X— is:

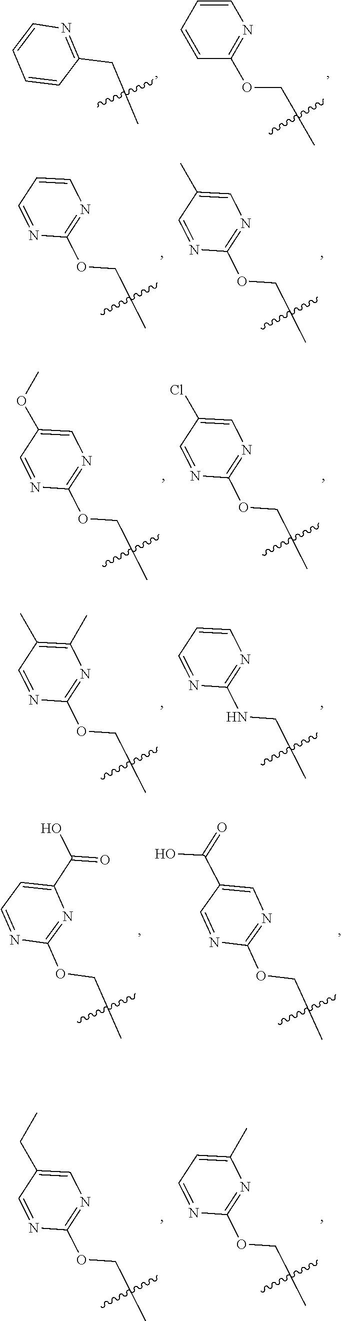

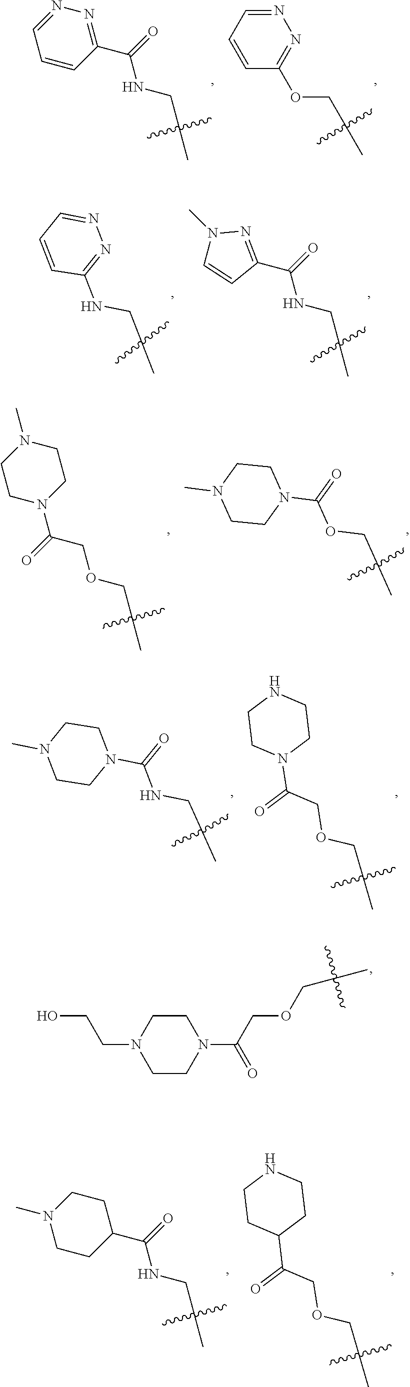

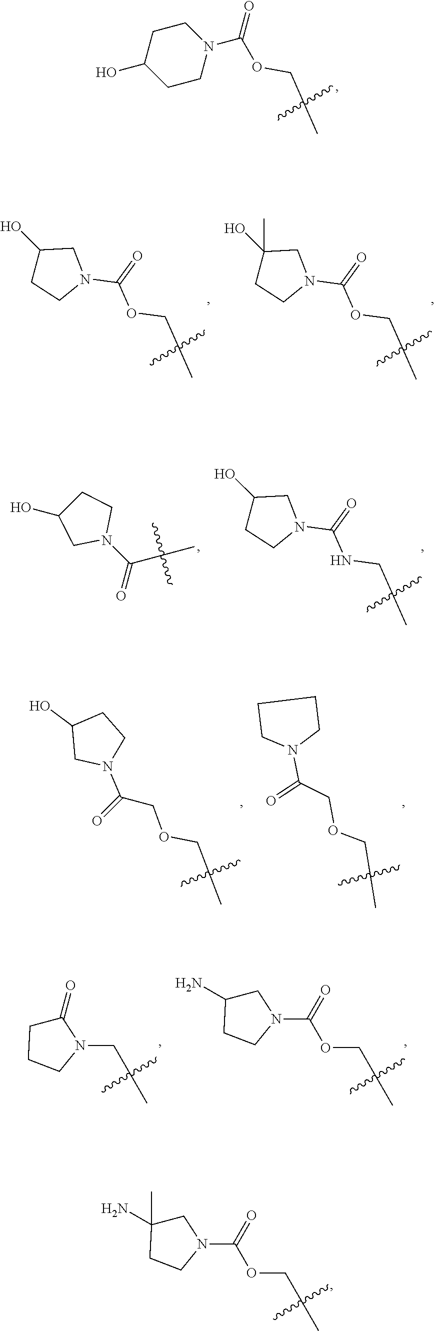

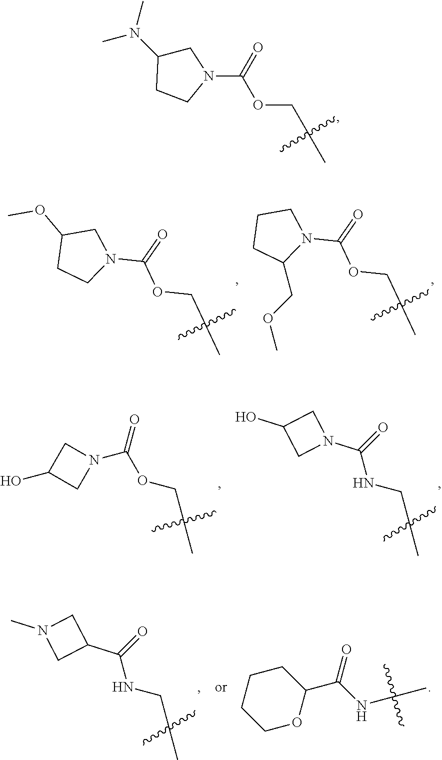

32. A compound selected from the compounds in Table 1, or a pharmaceutically acceptable salt thereof.

33. A compound selected from the compounds in Table 2, or a pharmaceutically acceptable salt thereof.

34. A pharmaceutical composition comprising the compound of any one of claims 1-33, or a pharmaceutically acceptable salt thereof, and at least one pharmaceutically acceptable excipient.

35. A method of modulating bis-phosphoglycerate mutase (BPGM) comprising contacting an effective amount of the compound of any one of claims 1-33, or a pharmaceutically acceptable salt thereof, or the pharmaceutical composition of claim 34, with the BPGM.

36. A method of treating sickle cell disease in a subject in need thereof, comprising administering to the subject a therapeutically effective amount of the compound of any one of claims 1-33, or a pharmaceutically acceptable salt thereof, or the pharmaceutical composition of claim 34.
